(19) United States
(12) Patent Application Publication
(10) Pub. No.: US 2003/0013851 A1
(43) Pub. Date: Jan. 16, 2003

(54) Solution structure of IL-13 and uses thereof

(76) Inventors: Robert Powers, Westfield, MA (US); Michelle Catino, Medford, MA (US); Chu-Lai Hsiao, Waltham, MA (US); Karl Malakian, Boxborough, MA (US); Franklin J. Moy, Arlington, MA (US); James M. Wilhelm, Boston, MA (US)

Correspondence Address: Craig J. Arnold, Amster, Rothstein & Ebenstein, 90 Park Avenue, New York, NY, 10016, US

(21) Appl. No.: 10/150,874

(22) Filed: May 16, 2002

Related U.S. Application Data
(60) Provisional application No. 60/296,607, filed on Jun. 7, 2001.

(51) Int. Cl. G06F 19/00; G01N 33/48; G01N 33/50; C07K 14/54

(52) U.S. Cl. 530/351; 702/19

(57) ABSTRACT

The present invention relates to the three dimensional solution structure of interleukin-13 (IL-13), as well as the identification and characterization of various binding active sites of IL-13. Also provided for by the present invention are methods of utilizing the three dimensional structure for the design and selection of potent and selective agents that interact with IL-13.

### CROSS REFERENCE TO RELATED APPLICATION

This application claims the benefit of U.S. Provisional Application No. 60/296,607 filed Jun. 7, 2001.

### FIELD OF THE INVENTION

The present invention relates to the three dimensional solution structure of human IL-13. This structure is critical for the design and selection of potent and selective agents that interact with IL-13.

### BACKGROUND OF THE INVENTION

Interleukin-13 (IL-13) is a pleiotropic cytokine with roles in atopy, asthma, allergy and inflammatory response (For reviews see: Corry, 1999; De Vries, 1998; Finkelman et al., 1999; Shirakawa et al., 2000; Wills-Karp et al., 1998). IL-13 is produced by activated T cells and promotes B cell proliferation, induces B cells to produce IgE, down regulates the production of proinflamatory cytokines, increases expression of VCAM-1 on endothelial cells, enhances the expression of class II MHC antigens and various adhesion molecules on monocytes. IL-13 mediates these functions through an interaction with its receptor on hematopoietic and other cell types, but currently no functional receptors have been identified on T cells. The signaling human IL-13 receptor (IL-13 R) is a heterodimer composed of the interleukin-4 receptor α chain (IL-4Rα) and the IL-13 binding chain. Two IL-13 binding domains that are 27% homologous have been identified, IL-13Rα1 and IL-13Rα2. IL-13Rα2 demonstrates an approximate 100-fold higher affinity for IL-13 relative to IL-13Rα1 in the absence of IL-4Rα, but has been identified only in the serum and urine of mice. The association of IL-13 with its receptor induces the activation of STAT6 (signal transducer and activation of transcription 6) and Janus-family kinase (JAK1, JAK2, TYK2) through a binding interaction with the IL-4Rα chain.

IL-13 is located in a cluster of genes on chromosome 5 encoding IL-3, IL-4, IL-5, IL-9 and GM-CSF. IL-13 shares many functional properties with IL-4 as a result of the common IL-4Rα component in their receptors (Callard et al., 1996; Gessner and Rollinghoff, 2000). IL-4 exhibits a high affinity to IL-4Rα chain (K_{d}=20-300 pM), where this complex recruits the common γ chain (γ_{c}) of IL-4R to form the signaling complex. Similarly, IL-13 binds to the IL-13 binding chain (IL-13 Rα1) with relatively high affinity (K_{d}˜4 nM) in the absence of the IL-4Rα chain, where an increase of affinity to IL-R occurs in the presence of IL-4Rα (K_{d}˜50 pM). IL-13 does not bind IL-4Rα in the absence of the IL-13 binding chain. As a result, IL-4 exhibits binding to both IL-4R and IL-13R due to the existence of the IL-4Rα chain in both receptors, but IL-13 does not bind IL-4R because of the absence of the IL-13 binding chain (Callard et al., 1996). The cross-reactivity of IL-4 with both IL-4R and IL-13R is further promoted by the antagonistic activity of the IL-4 Y124D mutant (De Vries, 1994). The IL-4 Y124D mutant still maintains the ability to bind IL-4Rα, but is deficient in its ability to induce a signal through interaction with the γ_{c} chain. Since the γ_{c} chain is not present in IL-13R, IL-13 does not induce the proliferation and differentiation of T cells or the activation of JAK-3 kinase, which associates with the γ_{c} chain of IL-4R.

IL-13 and IL-4 are both members of the short chain four-helix bundle cytokine family (Sprang and Bazan, 1993), where both solution and crystal structures have been previously determined for IL-4 (Powers et al., 1992; Powers et al., 1993; Smith et al., 1992; Walter et al., 1992; Wlodaver et al., 1992). Despite the relatively low (25%) sequence homology between IL-13 and IL-4, a similarity in the overall topology between the two proteins is expected. A combination of mutational and kinetic analysis has identified a distinct site on the IL-4 structure associated with IL-4Rα binding and a second site associated with signaling through the γ_{c} chain (Kruse et al., 1993; Letzelter et al., 1998; Wang et al., 1997). Recently, the X-ray structure of IL-4 complexed with the ectodomain of IL-4Rα has been determined, which further defines the IL-4-IL-4Rα interface (Hage et al., 1999).

Despite the abundance of structural information on the IL-4 receptor system, structural information for IL-13, IL-13R or the complex is currently lacking. The present invention provides a high-resolution solution structure of human IL-13 by heteronuclear multidimensional NMR.

### SUMMARY OF THE INVENTION

The present invention relates to the three dimensional structure of IL-13, and more specifically, to the solution structure of IL-13, as determined using spectroscopy and various computer modeling techniques.

Particularly, the invention is further directed to the identification, characterization and three dimensional structure of an active site of IL-13 that provides an attractive target for the rational design of potent and selective agents that interact with IL-13.

Accordingly, the present invention provides a solution comprising IL-13. The three dimensional solution structure of IL-13 is provided by the relative atomic structural coordinates of FIG. 8, as obtained from spectroscopy data.

Also provided by the present invention is an active site of IL-13, wherein said active site is characterized by a three dimensional structure comprising the relative structural coordinates of amino acid residues A9, E12, E15, E16 and M66 of IL-13 according to FIG. 8 or 9, ± a root mean square deviation from the conserved backbone atoms of said amino acids of not more than 1.5 Å.

Also provided for by the present invention is an active site of IL-13, wherein said active site is characterized by a three dimensional structure comprising the relative structural coordinates of amino acid residues 152, Q64, R65 and M66 of IL-13 according to FIG. 8 or 9, ± a root mean square deviation from the conserved backbone atoms of said amino acids of not more than 1.5 Å.

The solution coordinates of IL-13 or portions thereof (such as the active sites), as provided by this invention may be stored in a machine-readable form on a machine-readable storage medium, e.g. a computer hard drive, diskette, DAT tape, etc., for display as a three-dimensional shape or for other uses involving computer-assisted manipulation of, or computation based on, the structural coordinates or the three-dimensional structures they define. By way of example, the data defining the three dimensional structure of IL-13 as set forth in FIG. 8 or 9 may be stored in a machine-readable storage medium, and may be displayed as a graphical three-dimensional representation of the relevant structural coordinates, typically using a computer capable of reading the data from said storage medium and programmed with instructions for creating the representation from such data.

Accordingly, the present invention provides a machine, such as a computer, programmed in memory with the coordinates of IL-13 or portions thereof, together with a program capable of converting the coordinates into a three dimensional graphical representation of the structural coordinates on a display connected to the machine. A machine having a memory containing such data aids in the rational design or selection of inhibitors of IL-13 binding or activity, including the evaluation of the ability of a particular chemical entity to favorably associate with IL-13 as disclosed herein, as well as in the modeling of compounds, proteins, complexes, etc. related by structural or sequence homology to IL-13.

The present invention is additionally directed to a method of determining the three dimensional structure of a molecule or molecular complex whose structure is unknown, comprising the steps of first obtaining crystals or a solution of the molecule or molecular complex whose structure is unknown, and then generating X-ray diffraction data from the crystallized molecule or molecular complex and/or generating NMR data from the solution of the molecule or molecular complex. The generated diffraction or spectroscopy data from the molecule or molecular complex can then be compared with the solution coordinates or three dimensional structure of IL-13 as disclosed herein, and the three dimensional structure of the unknown molecule or molecular complex conformed to the IL-13 structure using standard techniques such as molecular replacement analysis, 2D, 3D and 4D isotope filtering, editing and triple resonance NMR techniques, and computer homology modeling. Alternatively, a three dimensional model of the unknown molecule may be generated by generating a sequence alignment between IL-13 and the unknown molecule, based on any or all of amino acid sequence identity, secondary structure elements or tertiary folds, and then generating by computer modeling a three dimensional structure for the molecule using the three dimensional structure of, and sequence alignment with, IL-13.

The present invention further provides a method for identifying an agent that interacts with IL-13, comprising the steps of: (a) generating a three dimensional model of IL-13 using the relative structural coordinates of the amino acids of FIG. 8 or 9, ± a root mean square deviation from the conserved backbone atoms of said amino acids of not more than 1.5 Å; and (b) employing said three-dimensional model to design an agent that interacts with IL-13.

Finally, the present invention provides agents that designed or selected using the methods disclosed herein. Additional objects of the present invention will be apparent from the description which follows.

### BRIEF DESCRIPTION OF THE FIGURES

FIG. 1 represents strip plots taken from the CBCA(CO)NH and CBCANH spectra for the amides of residues E61 through F70 of IL-13. Each amide correlates with the C^{α} and C^{β} of the preceding residue in the CBCA(CO)NH spectra and with both its intraresidue C^{α} and C^{β} and the C^{α} and C^{β} of the preceding residue in the CBCANH spectra. Interresidue (i−1) correlations are indicated with the observed interresidue connectivities marked by a solid line. Negative contours are indicated by dashed lines.

FIG. 2 is a summary of the sequential and medium range NOEs involving the NH, H^{α} and H^{β} protons, the amide exchange and ³J_{HNα} coupling constant data, and the ¹³C^{α} and ¹³C^{β} secondary chemical shifts observed for IL-13 with the secondary structure deduced from this data. The thickness of the lines reflects the strength of the NOEs. Amide protons still present after exchange to D₂O are indicated by closed circles. The open boxes on the same line as the H^{α}(i)−NH(i+1) NOEs represents the sequential NOE between the H^{α} proton of residue i and the C^{δ}H proton of the i+1 proline and is indicative of a trans proline.

FIG. 3 is a best-fit superposition of the backbone atoms (N,C,C′) of the 30 best structures determined for IL-13 for residues 1-113. The helices are shown as dark grey. The two disulfide bonds are shown between residues C29 and C57, and C45 and C71, respectively.

FIG. 4(a) is a ribbon diagram of the NMR structure of IL-13 colored by secondary structure (same view as FIG. 2). FIG. 4(c) is a ribbon diagram of the NMR structure of IL-4 (1BBN) (Powers et al., 1992; Powers et al., 1993). The view is the same as IL-13 based on the alignment of the common secondary structure elements and disulfide bonds. FIGS. 4(b) and 4(d) represent the top view of the IL-13 and IL-4 NMR ribbon diagram, respectively, illustrating the helix packing and orientation. The secondary structure elements and cysteines involved in disulfide bonds are labeled and are similar to FIG. 2.

FIG. 5(a) is the best-fit superposition of the backbone atoms (N, C, C′) of the IL-13 and IL-4 restrained minimized average NMR structures (Powers et al., 1992; Powers et al., 1993). FIG. 5(b) is the sequence alignment of IL-13 with IL-4 based on the common secondary structure elements and disulfide bonds. The IL-4 mutational data and residues involved in the IL-4Rα binding site based on the IL-4/IL4Rα X-ray structure (PDB ID:11AR) (Hage et al., 1999) are indicated on top of the sequence. The IL-13 mutational data is indicated on the bottom of the sequence. IL-4 residues involved in the IL-4Rα and the _{γC} binding sites identified by mutational analysis are labeled with (*) and (+), respectively. IL-4 residues identified as part of the IL-4Rα binding site from the X-ray structure without corresponding mutational data are labeled with (−). IL-13 residues involved in the IL-4Rα and the IL-13 binding chain binding sites identified by mutational analysis are labeled with (#) and (&), respectively. The IL-4 sequence numbering is on top and the IL-13 sequence numbering is on the bottom.

FIGS. 6(a) and 6(b) represent a GRASP molecular surface of the IL-4 and IL-13 NMR structures, respectively, where residues identified from mutational analysis that correlate with IL-4Rα affinity are shown. Residues proposed to interact with either the _{γC} or IL-13 binding chain (BC) are also shown. Residues in the IL-4Rα binding sites that were mutated are labeled.

FIG. 7(a) represents the IL-13/IL-4Rα model based on the IL-4/IL-4Rα X-ray structure (Hage et al., 1999). IL-13 replaced IL-4 in the IL-4/IL-4Rα X-ray structure by overlaying IL-13 onto IL-4 based on the common secondary structure elements and cysteins (see FIG. 4). IL-4Rα is shown as a molecular surface and IL-13 as a ribbon diagram, where the helices are labeled as A, B, C and D. Only the IL-13/IL-4Rα interface is illustrated. The secondary structure elements are labeled. FIG. 7(b) is an expanded view of the IL-13/IL-4Rα binding site indicating the interaction with helix α_{A} from IL-13. FIG. 7(c) is an expanded view of the IL-13/IL-4Rα binding site illustrating the interaction with helix α_{C} from IL-13. The side-chains for critical residues based on the IL-4/IL-4Rα X-ray structure and mutational data are shown and labeled. Residues from IL-4Rα are labeled with the prefix ‘r’.

FIG. 8 lists the atomic structure coordinates for the restrained minimized mean structure of IL-13 as derived by multidimensional NMR spectroscopy. “Atom type” refers to the atom whose coordinates are being measured. “Residue” refers to the type of residue of which each measured atom is a part—i.e., amino acid, cofactor, ligand or solvent. The “x, y and z” coordinates indicate the Cartesian coordinates of each measured atom's location (Å).

FIG. 9 provides the coordinates of the IL-13/IL-4Rα receptor model. “Atom type” refers to the atom whose coordinates are being measured. “Residue” refers to the type of residue of which each measured atom is a part—i.e., amino acid, cofactor, ligand or solvent. The “x, y and z” coordinates indicate the Cartesian coordinates of each measured atom's location (Å).

### DETAILED DESCRIPTION OF THE INVENTION

As used herein, the following terms and phrases shall have the meanings set forth below:

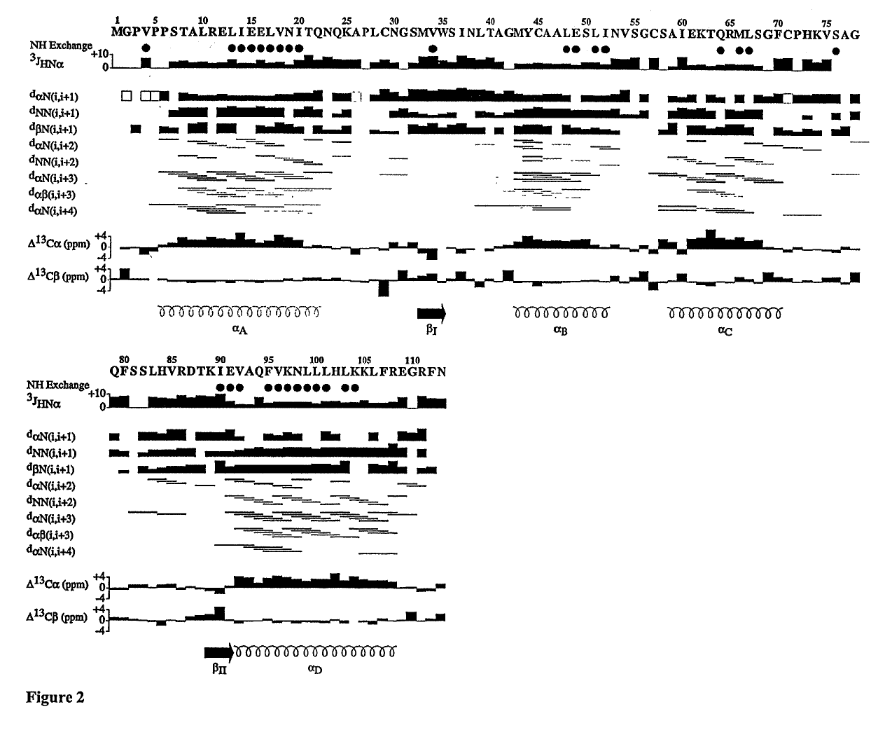

Unless otherwise noted, “IL-13” includes the amino acid sequence of FIG. 2, including conservative substitutions thereof.

Unless otherwise indicated, “protein” or “molecule” shall include a protein, protein domain, polypeptide or peptide.

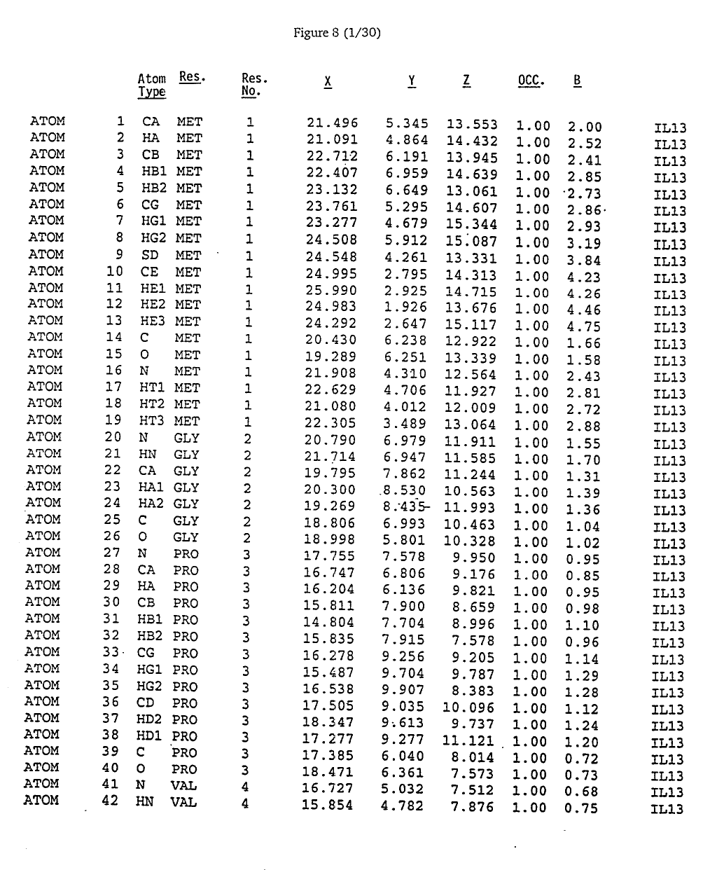

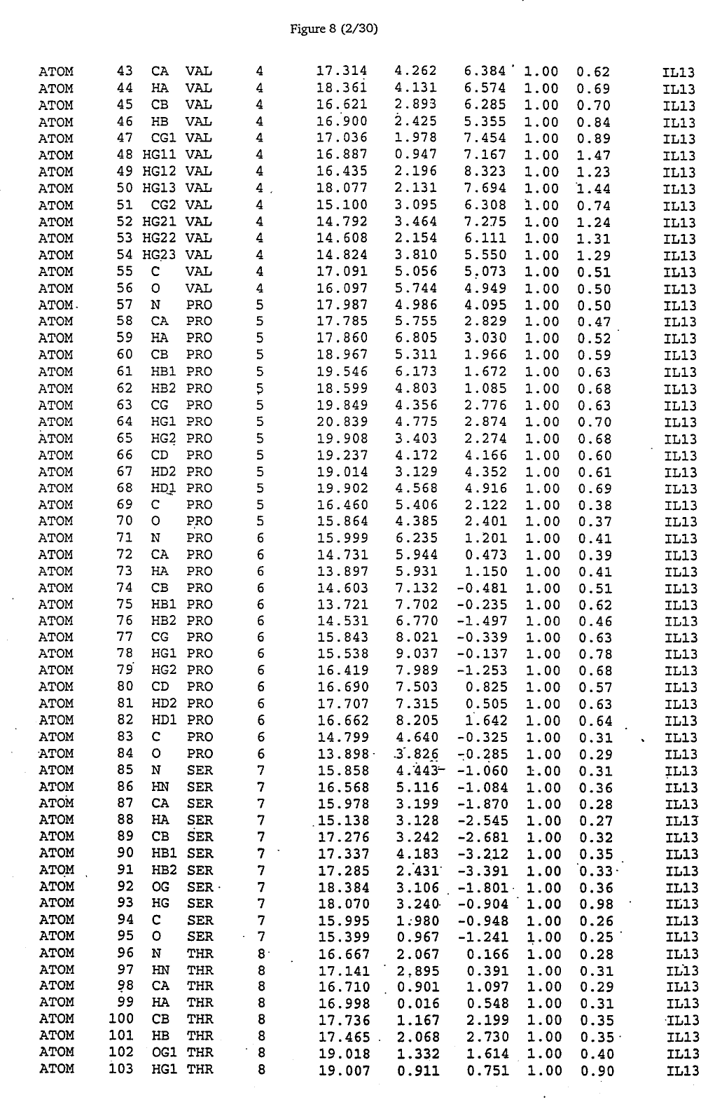

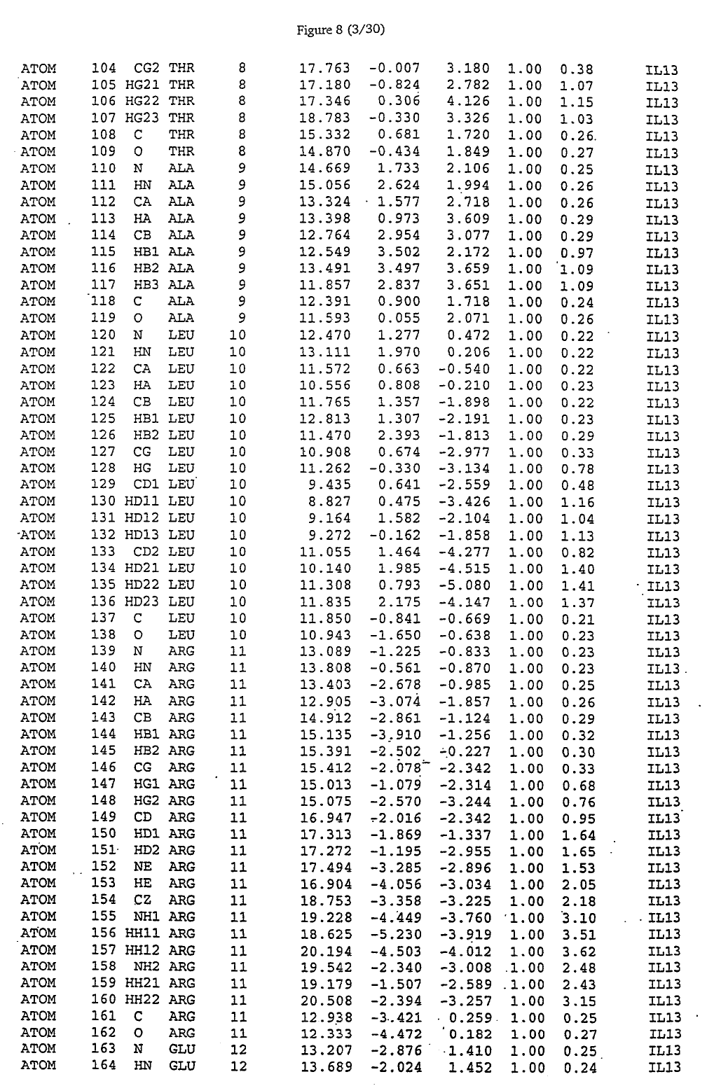

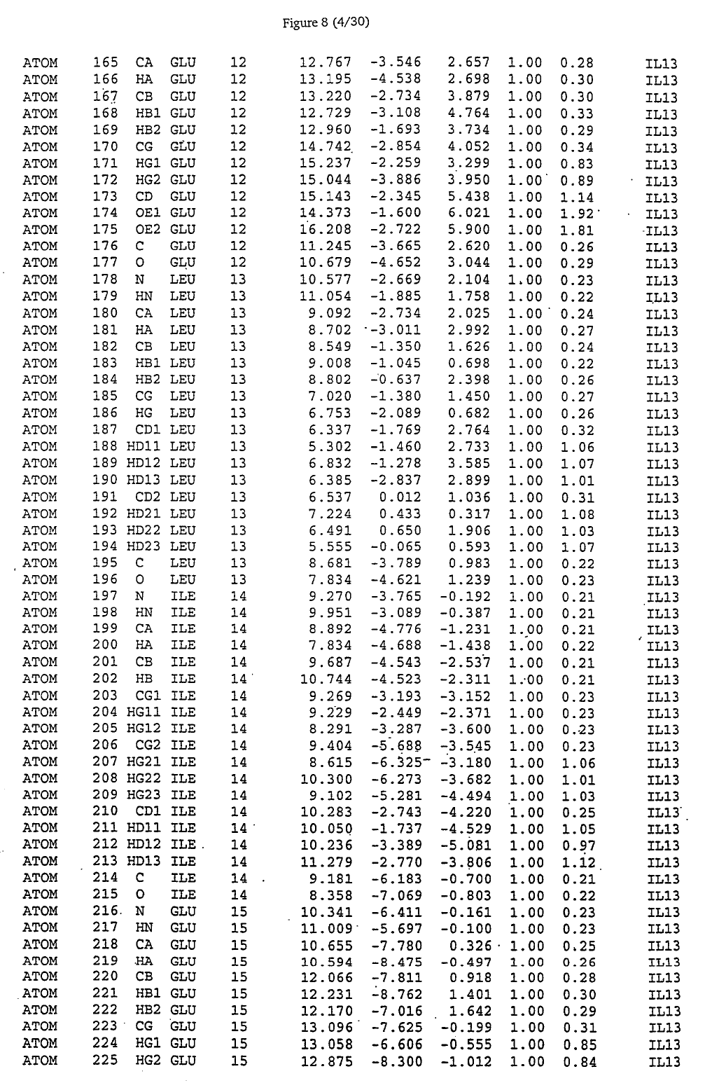

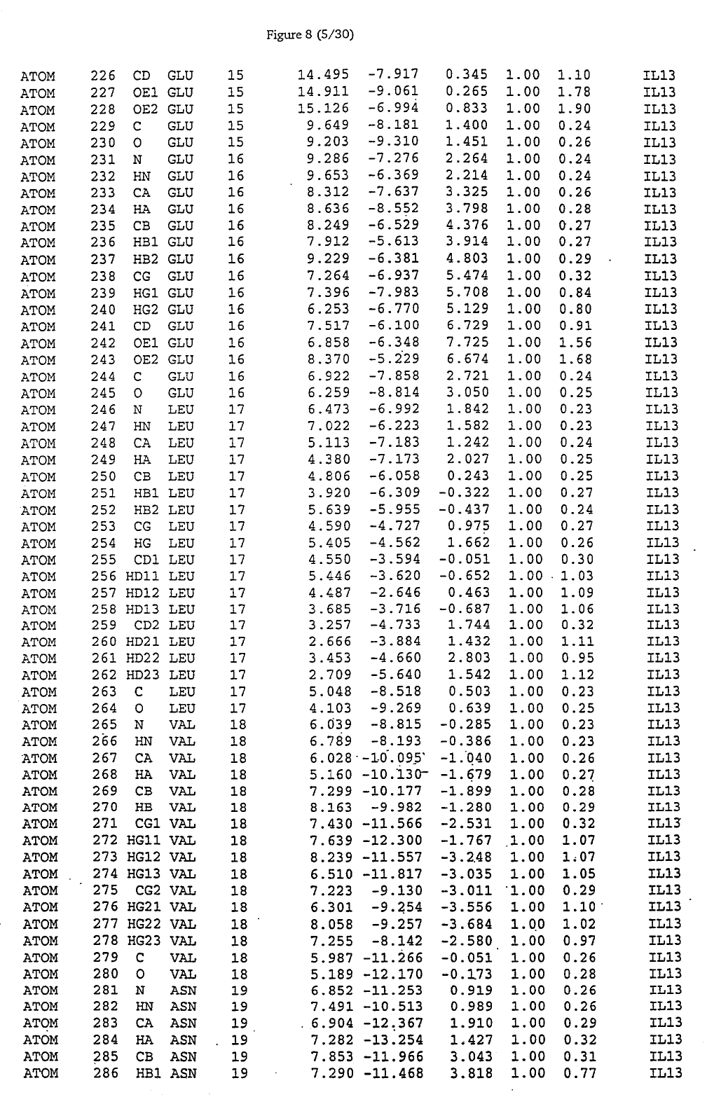

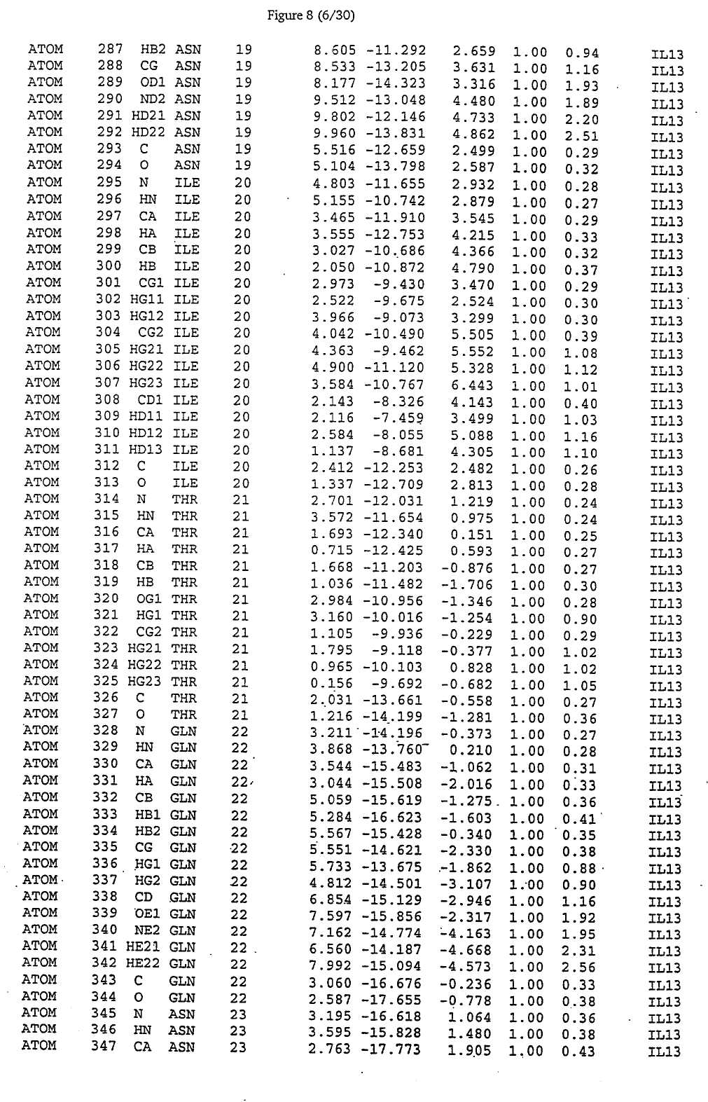

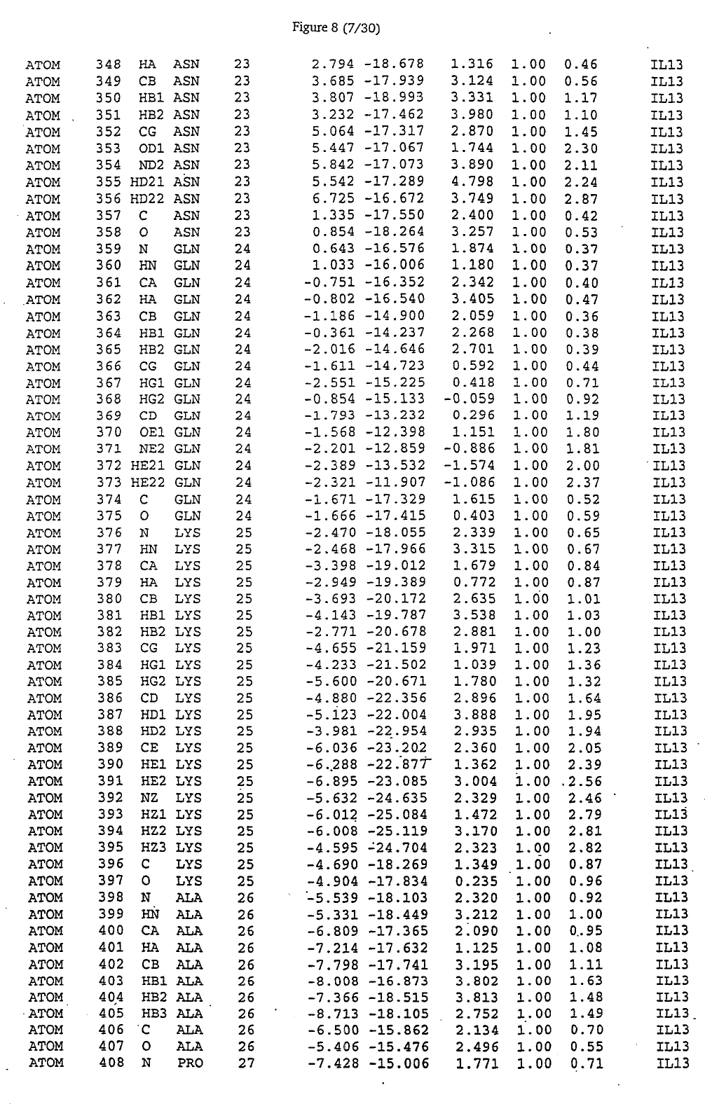

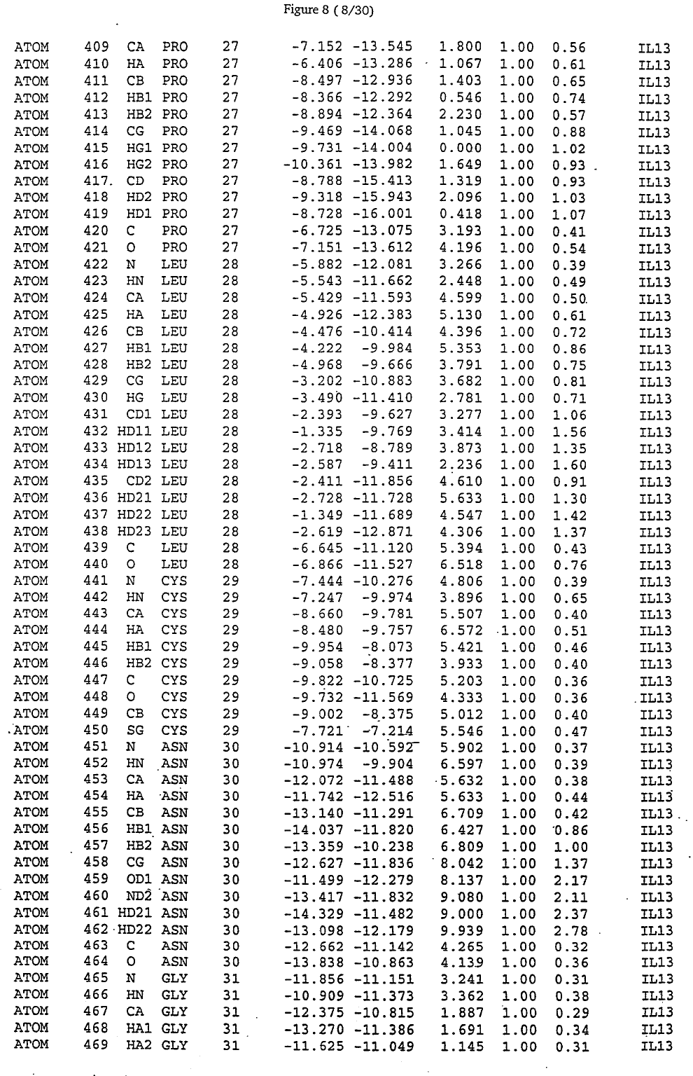

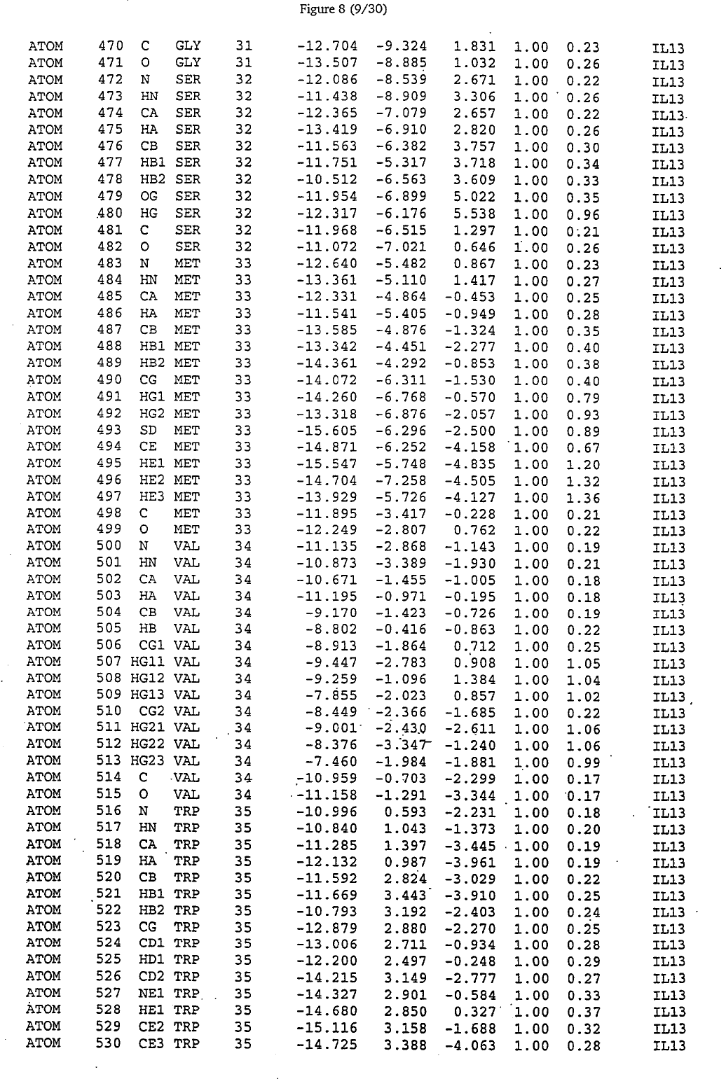

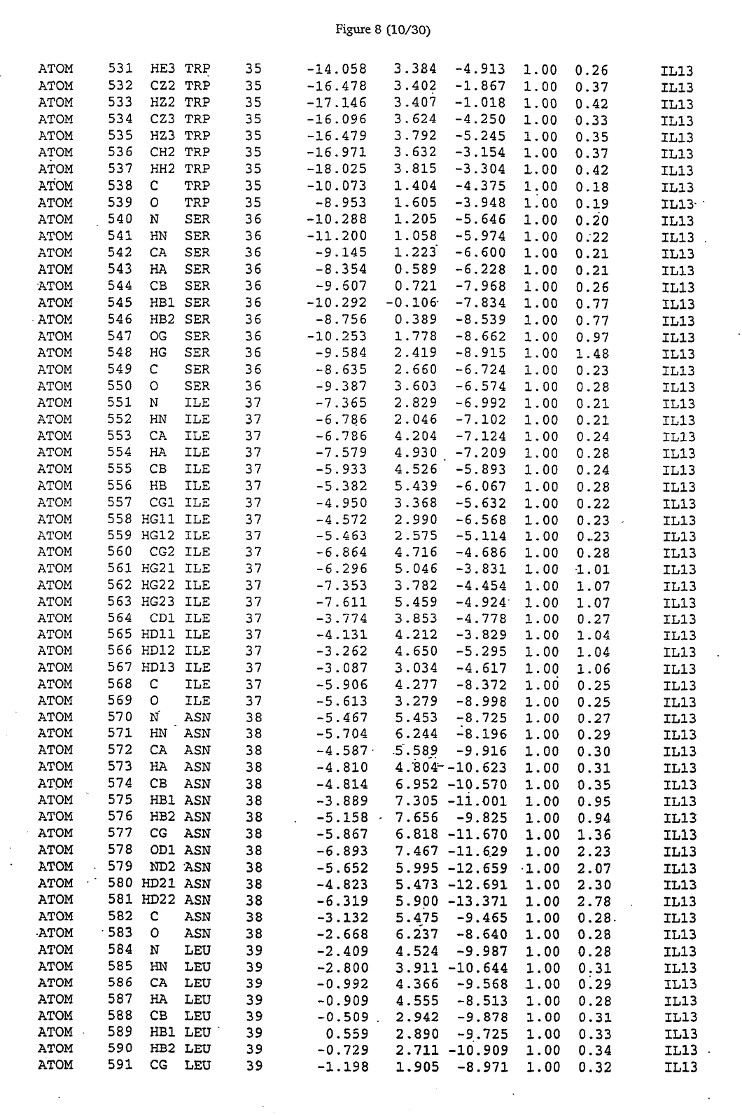

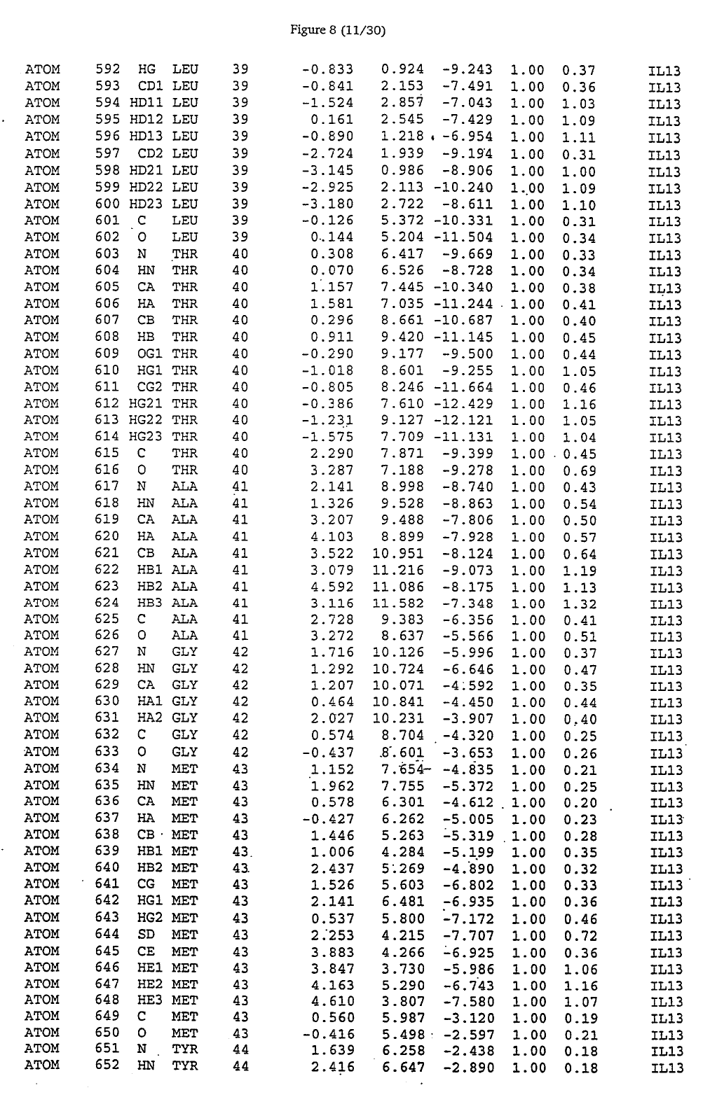

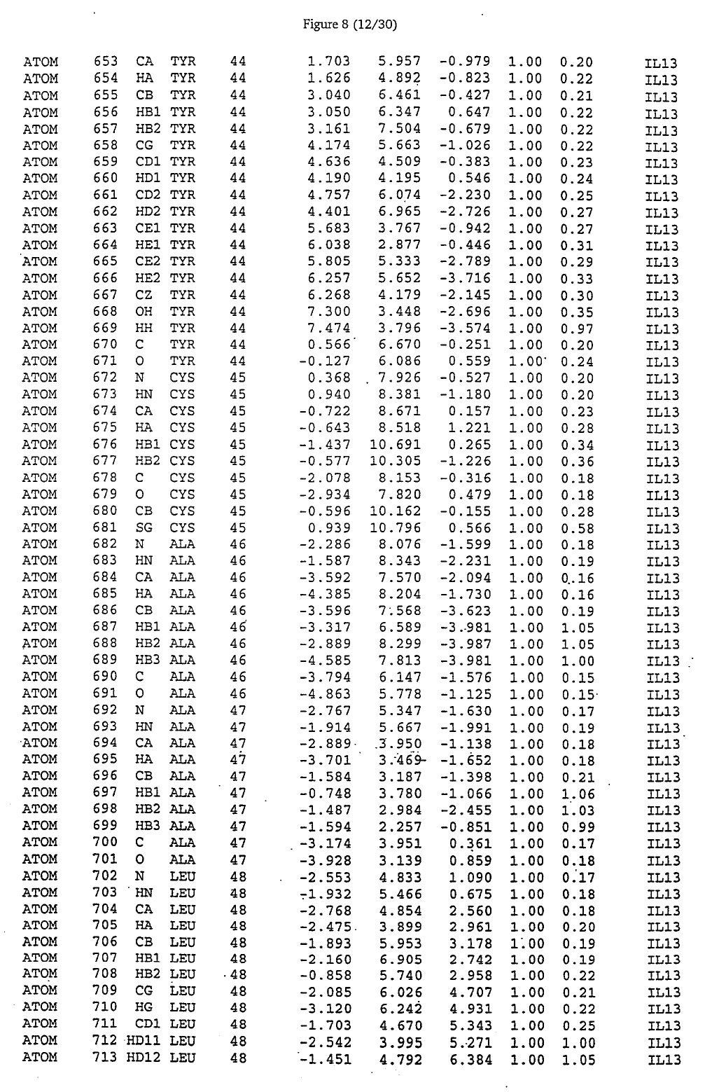

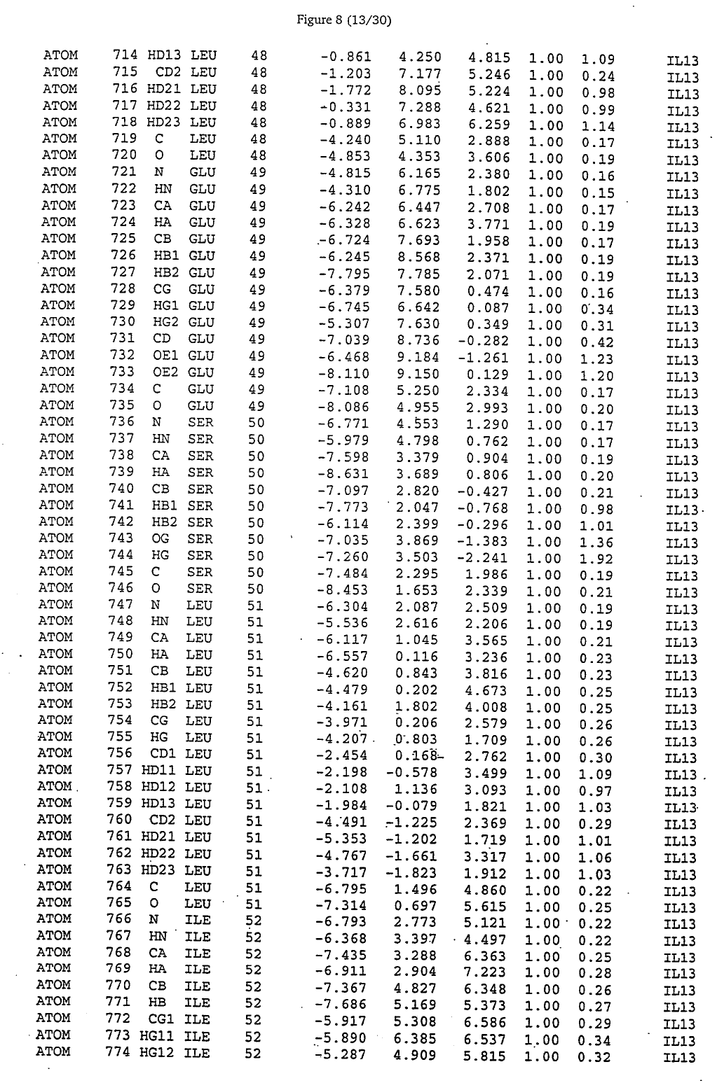

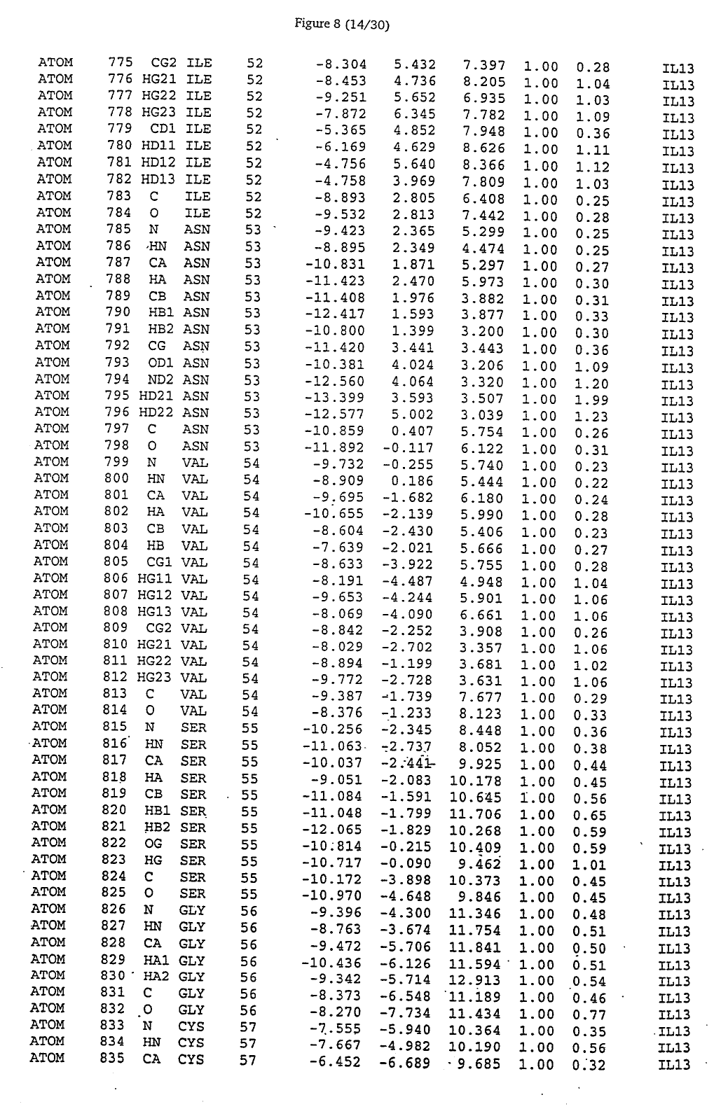

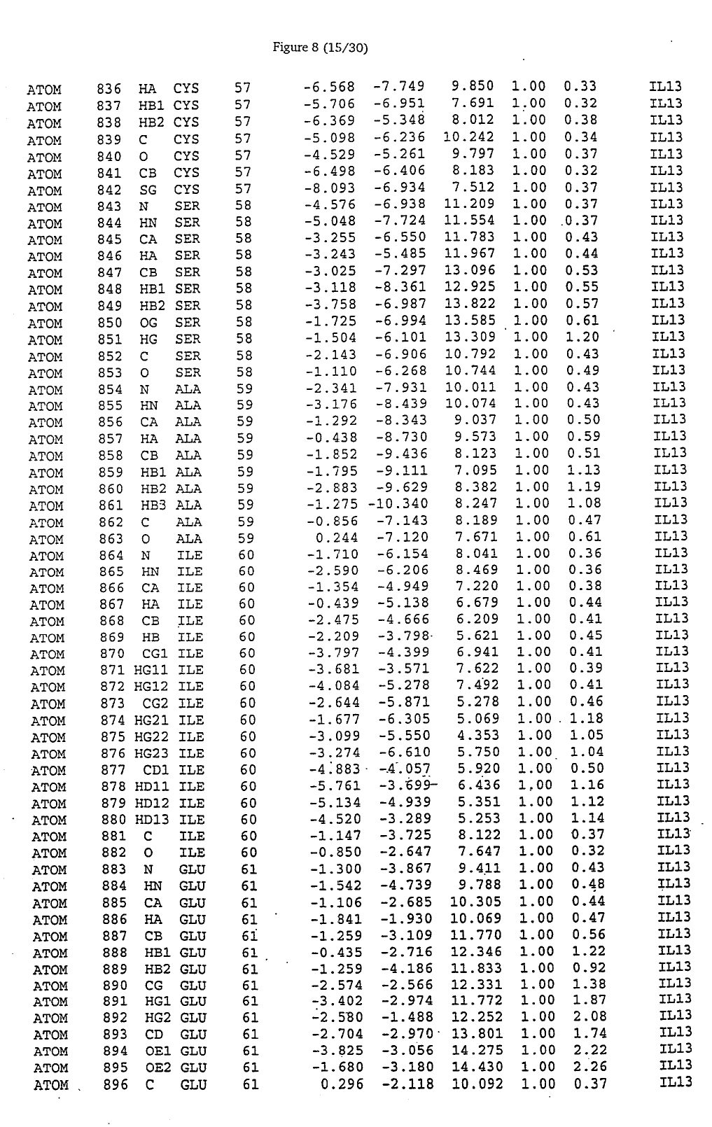

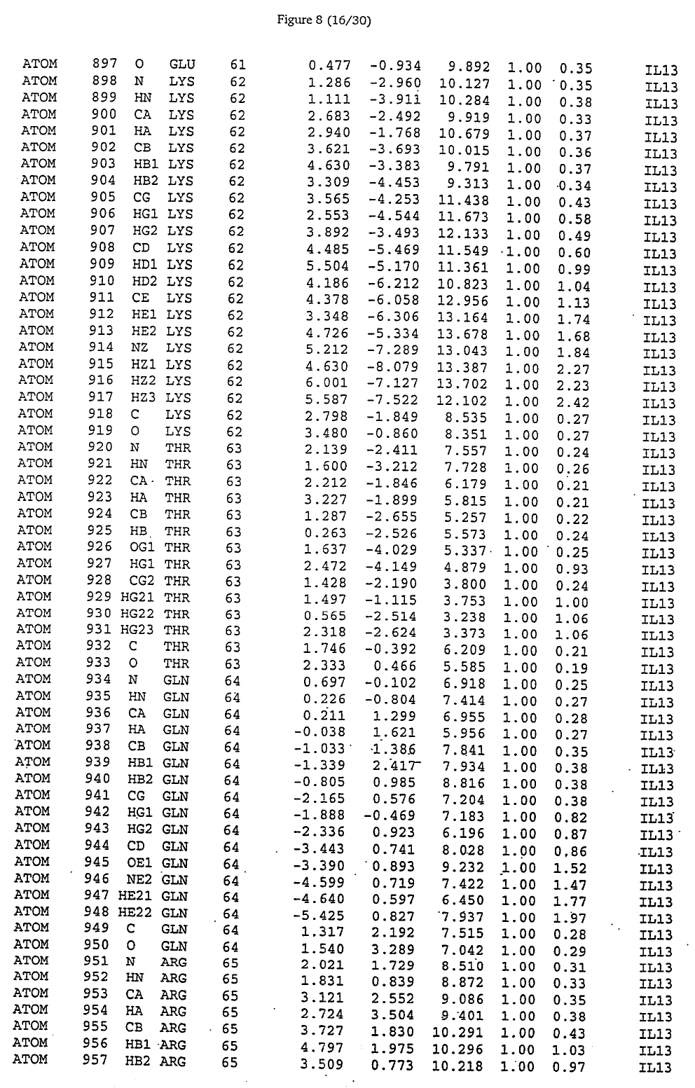

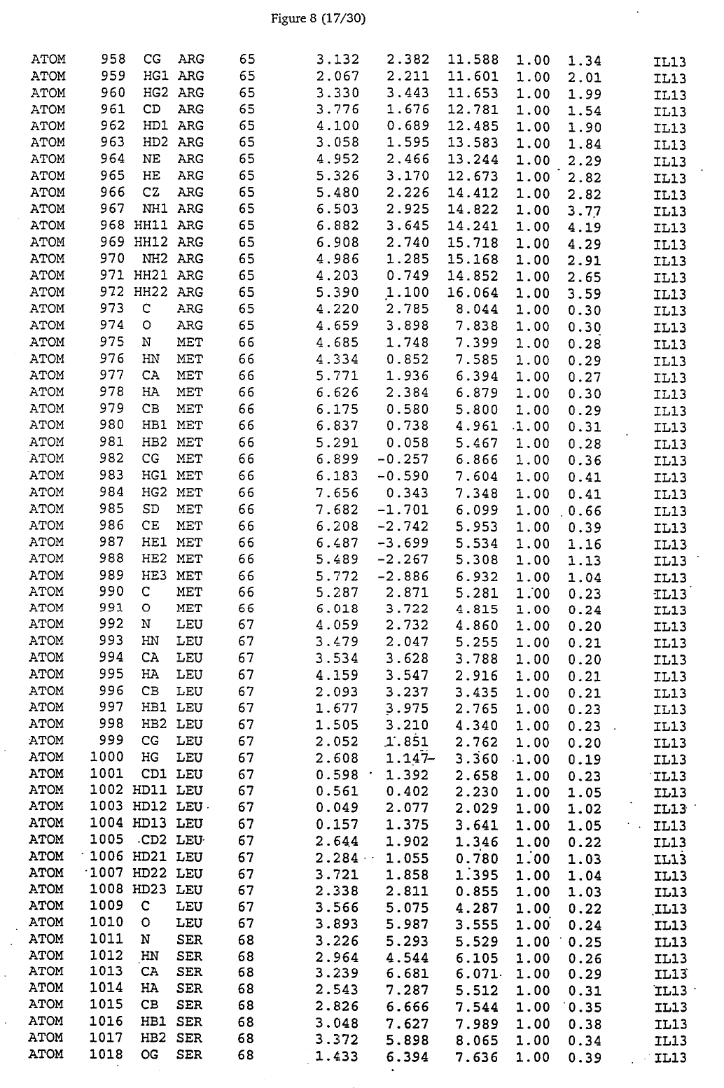

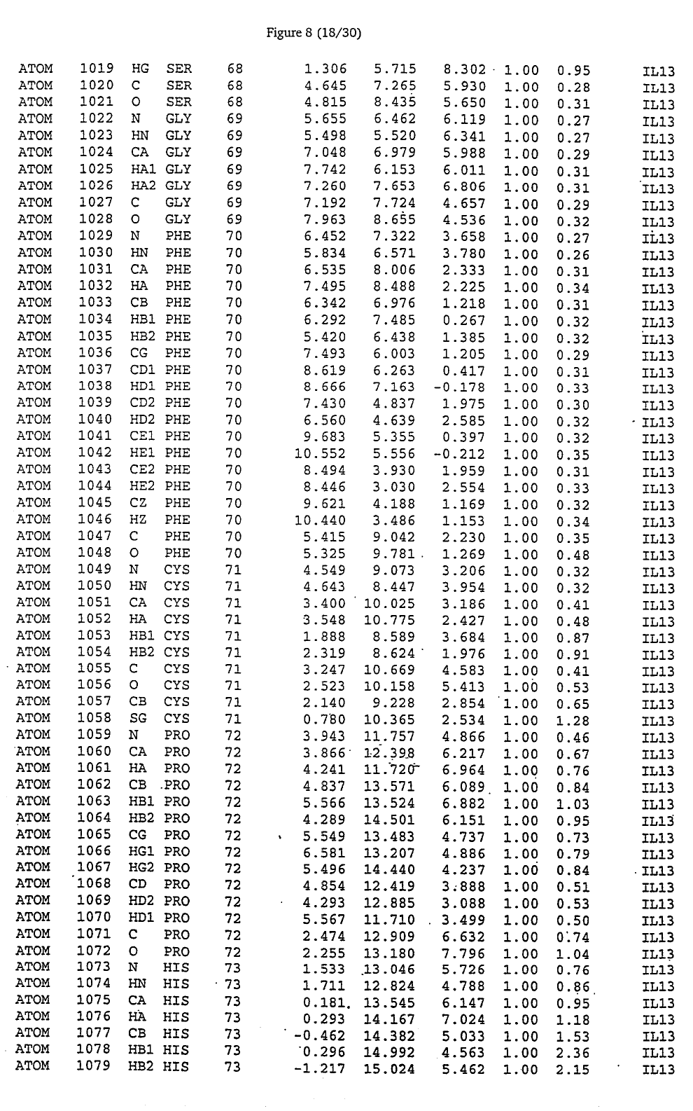

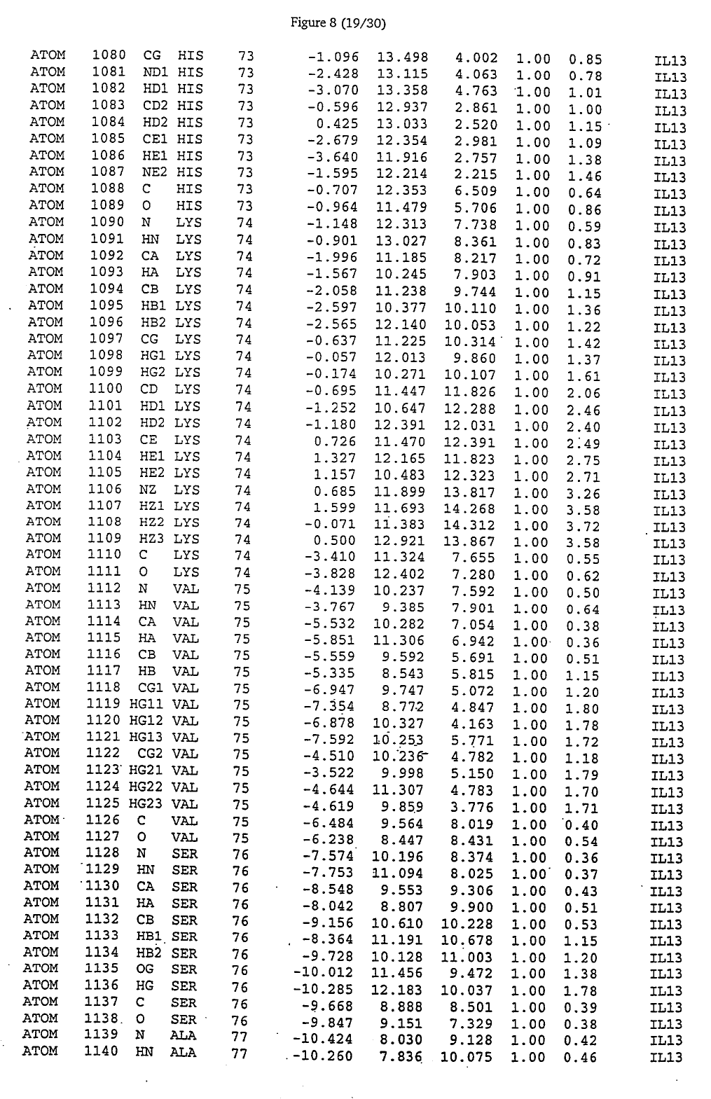

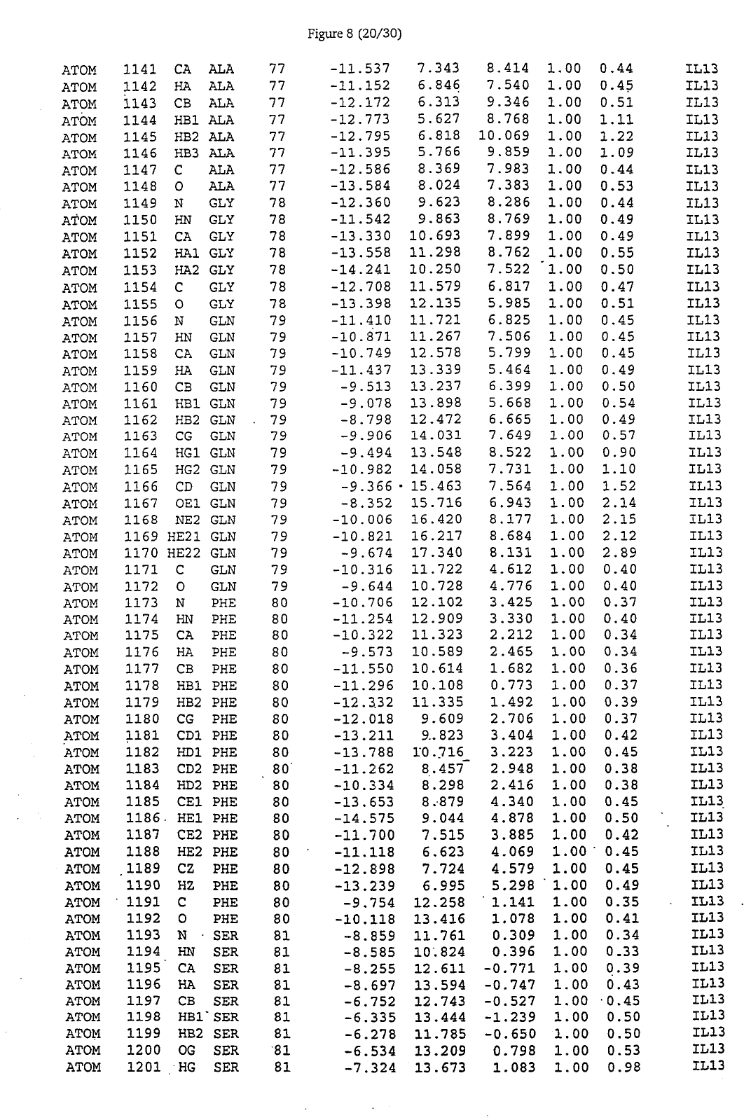

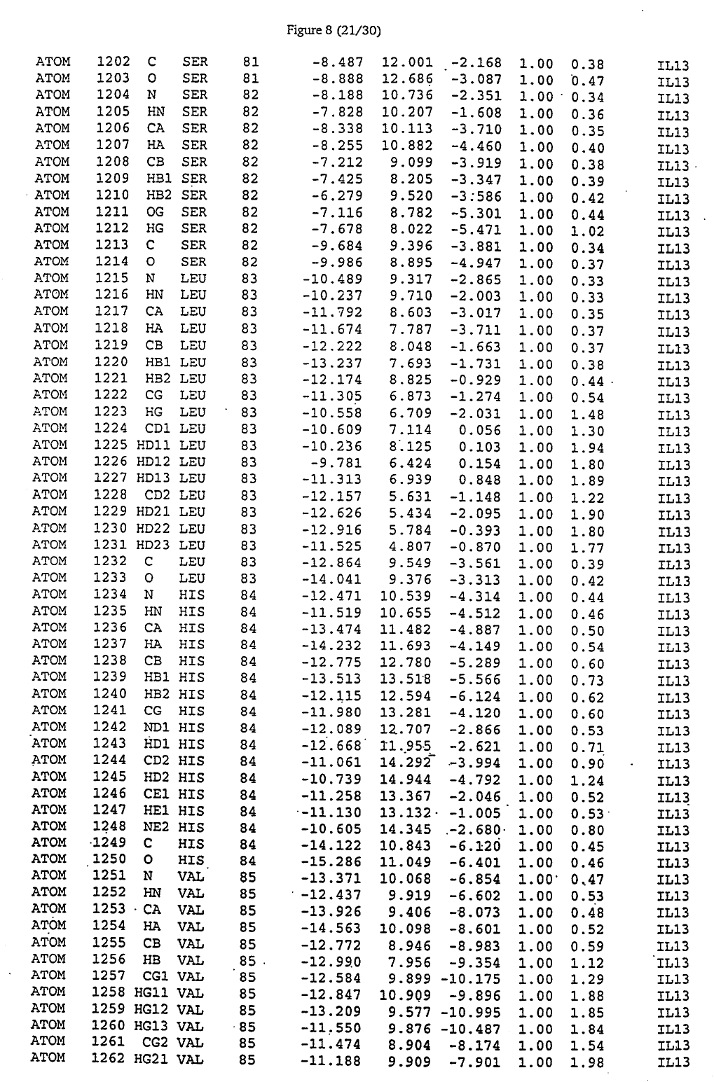

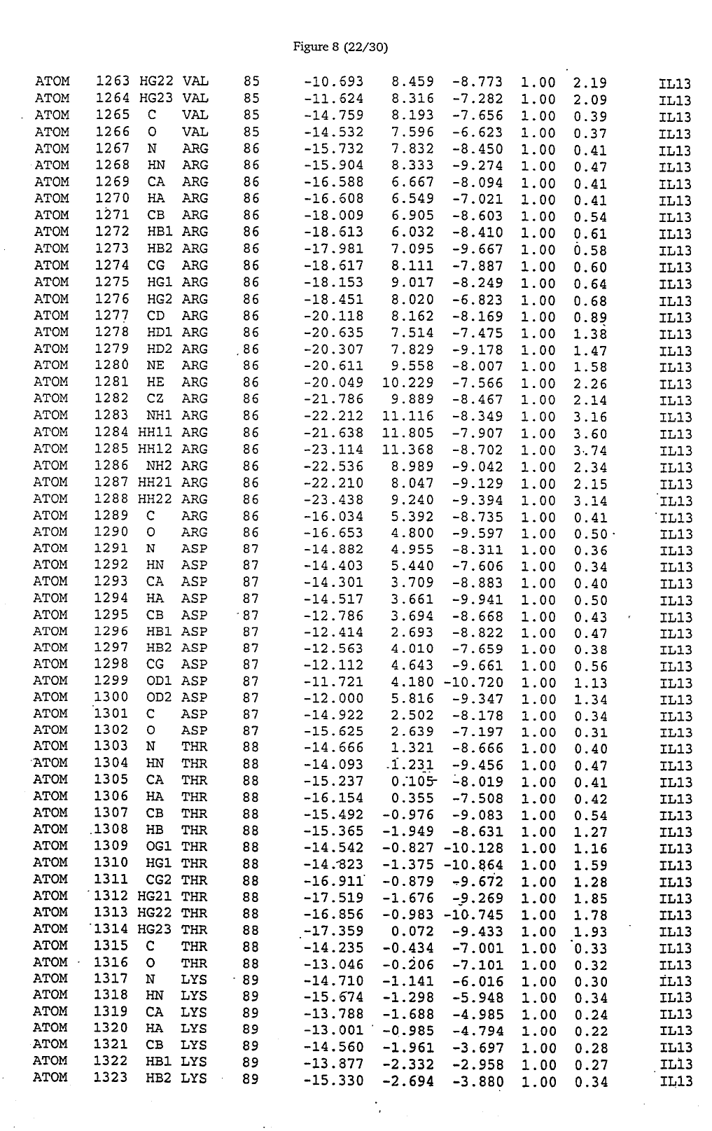

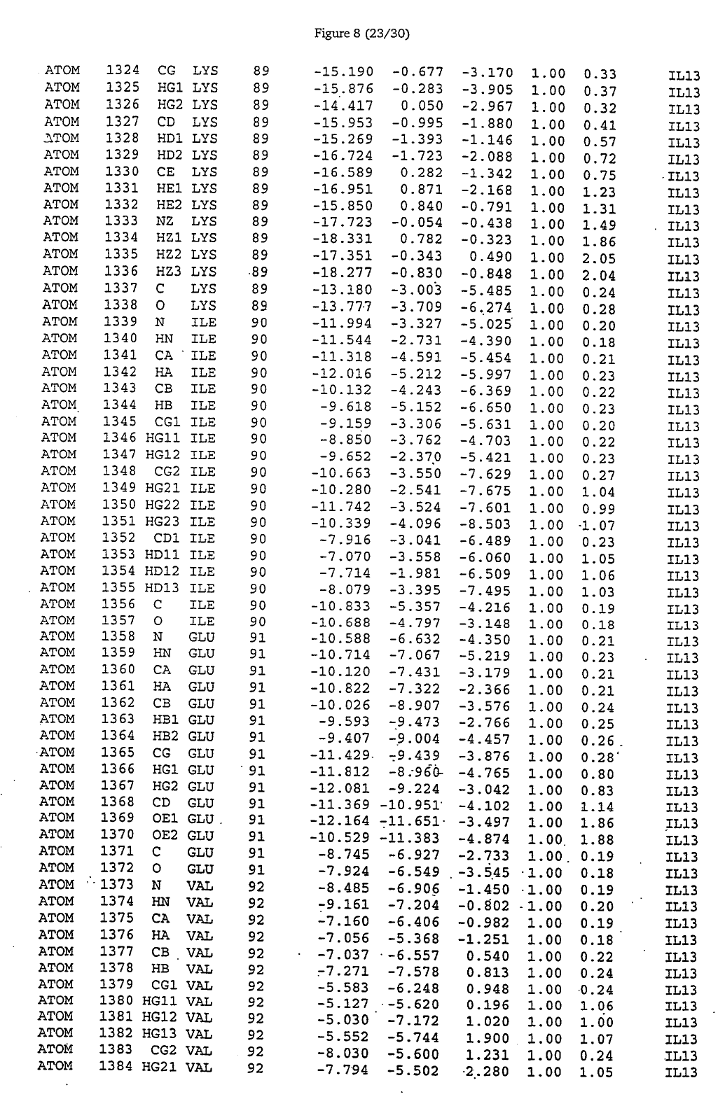

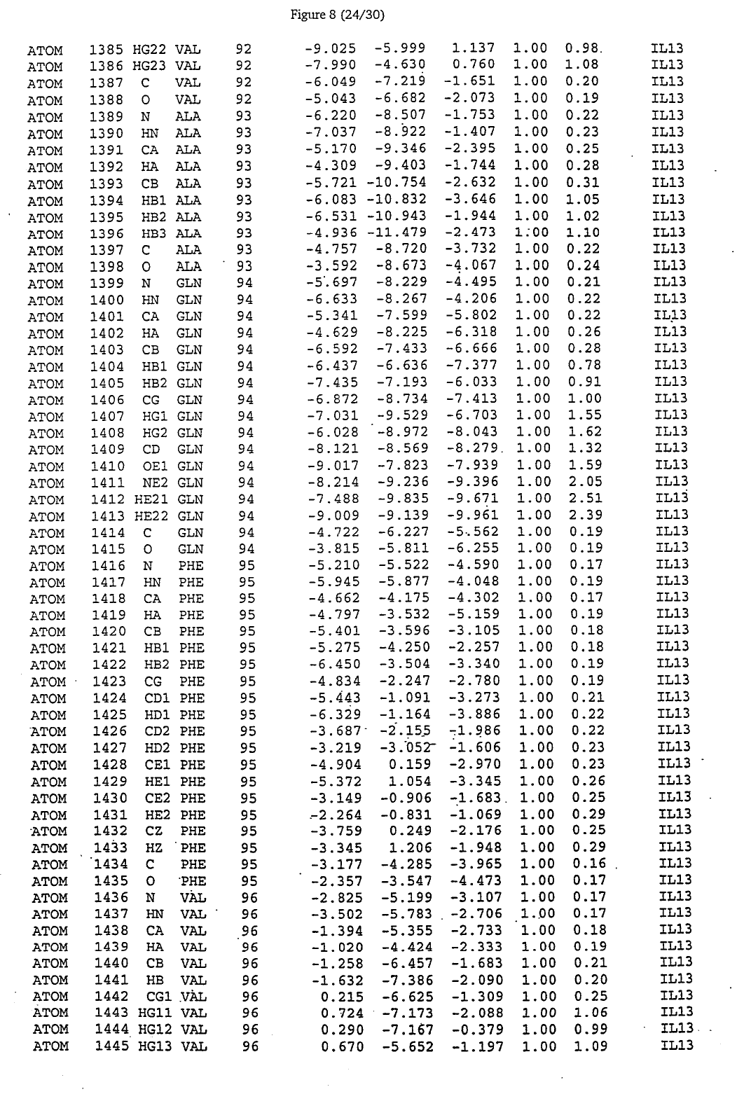

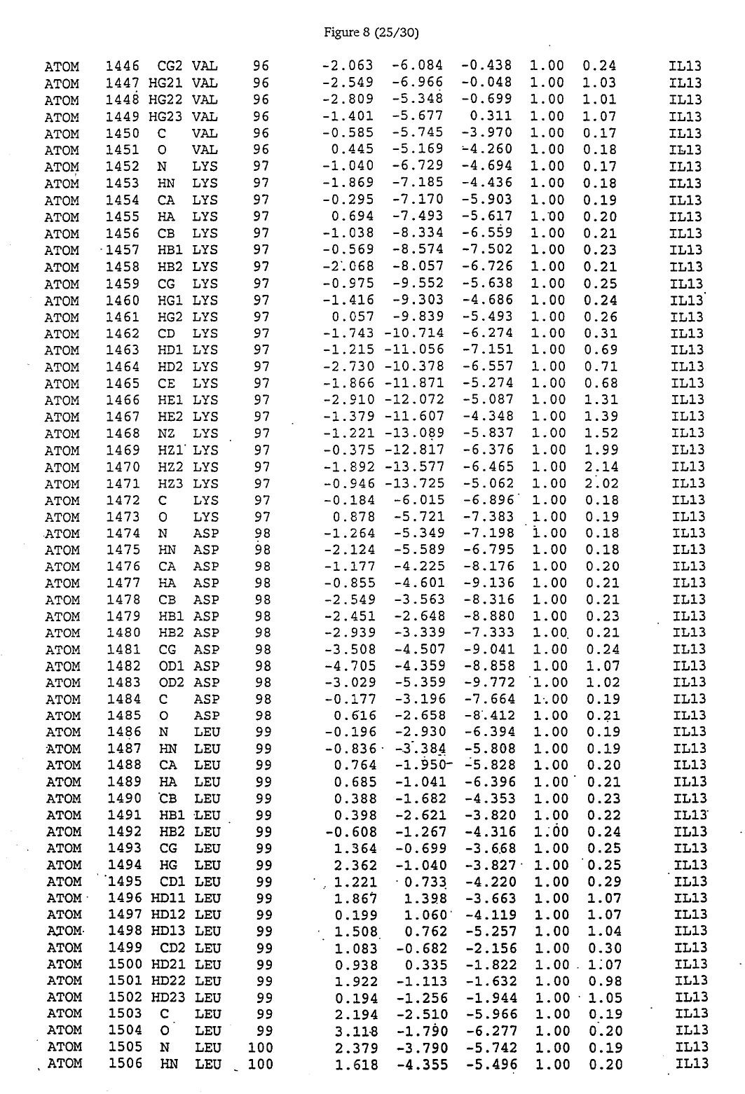

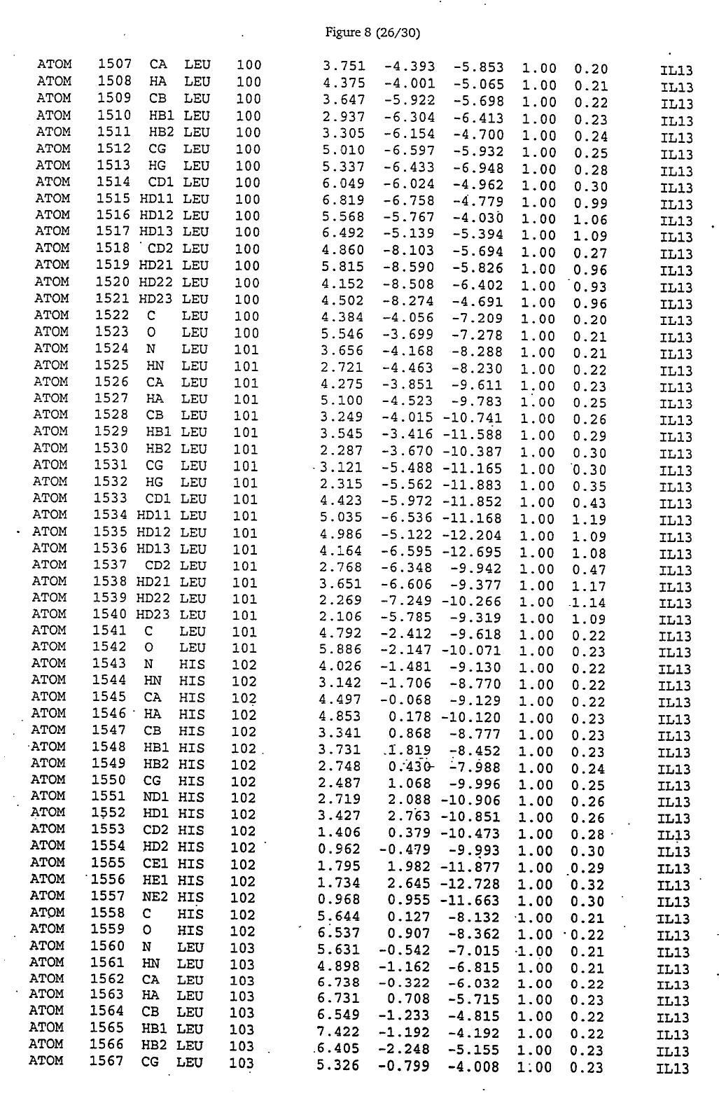

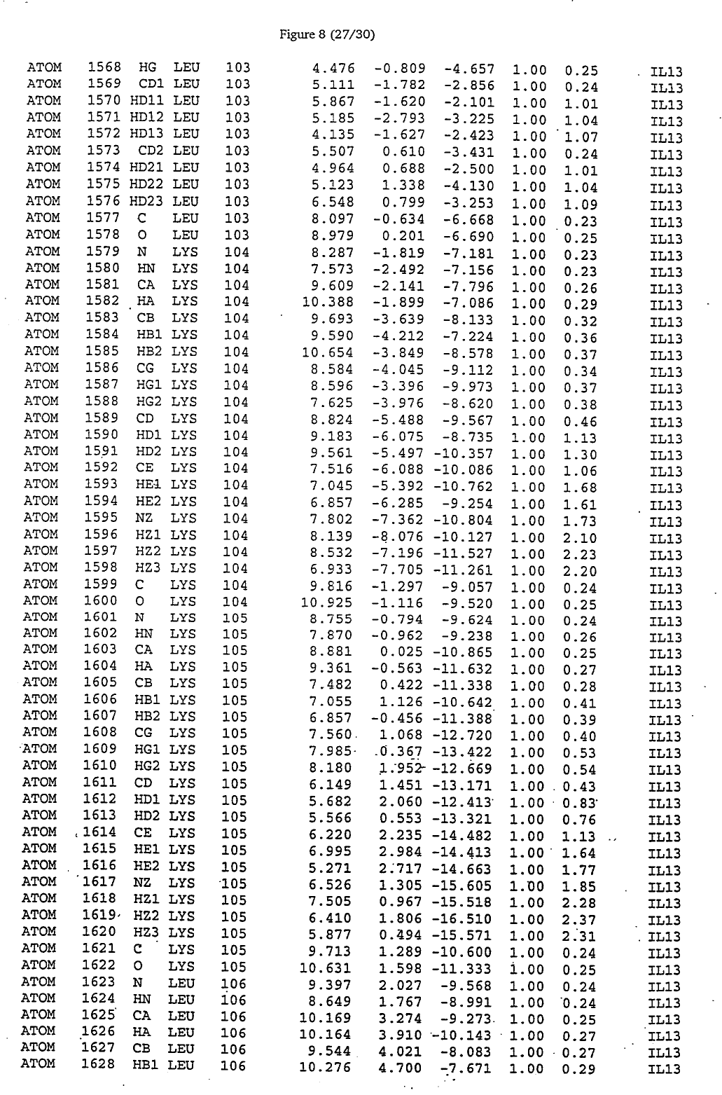

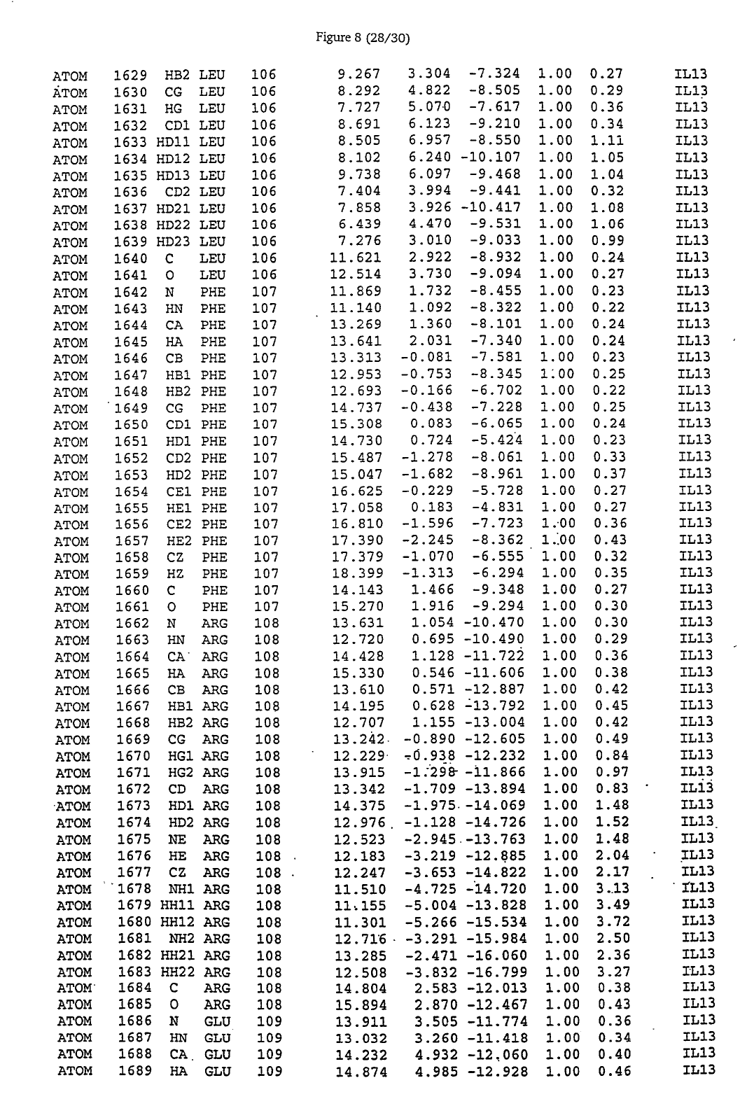

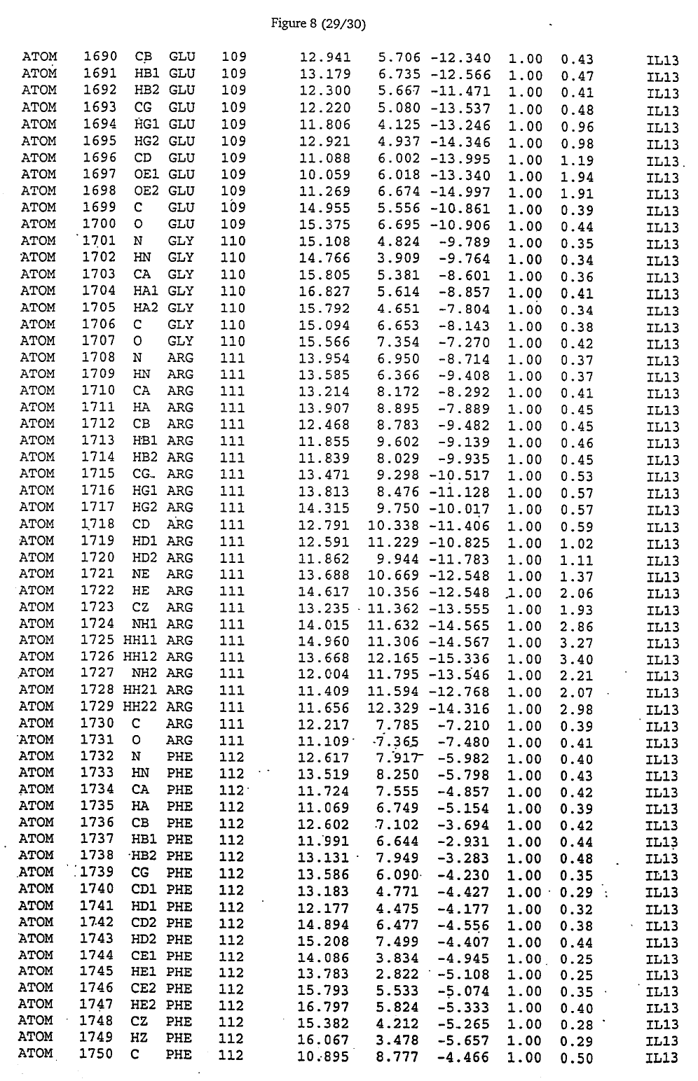

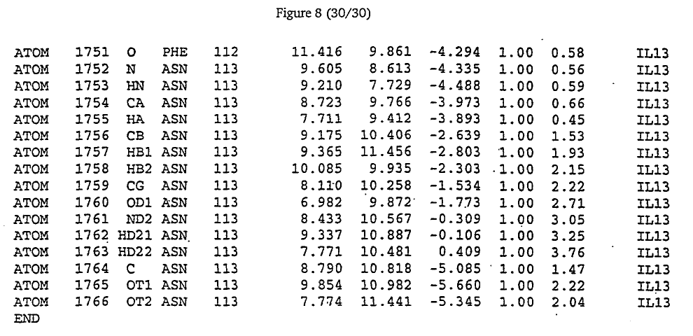

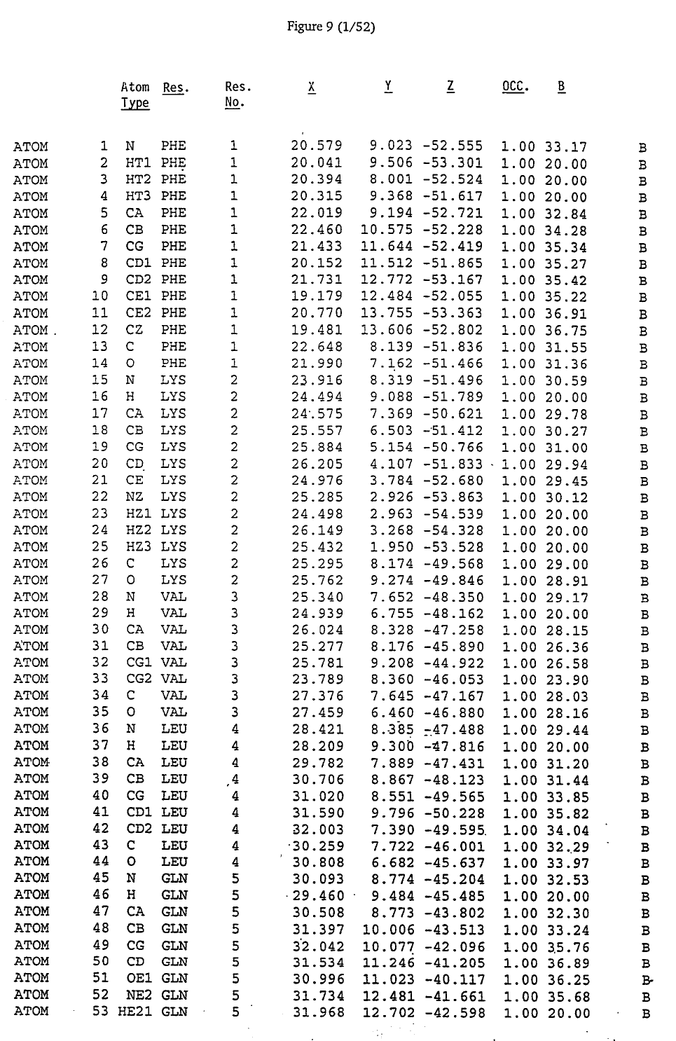

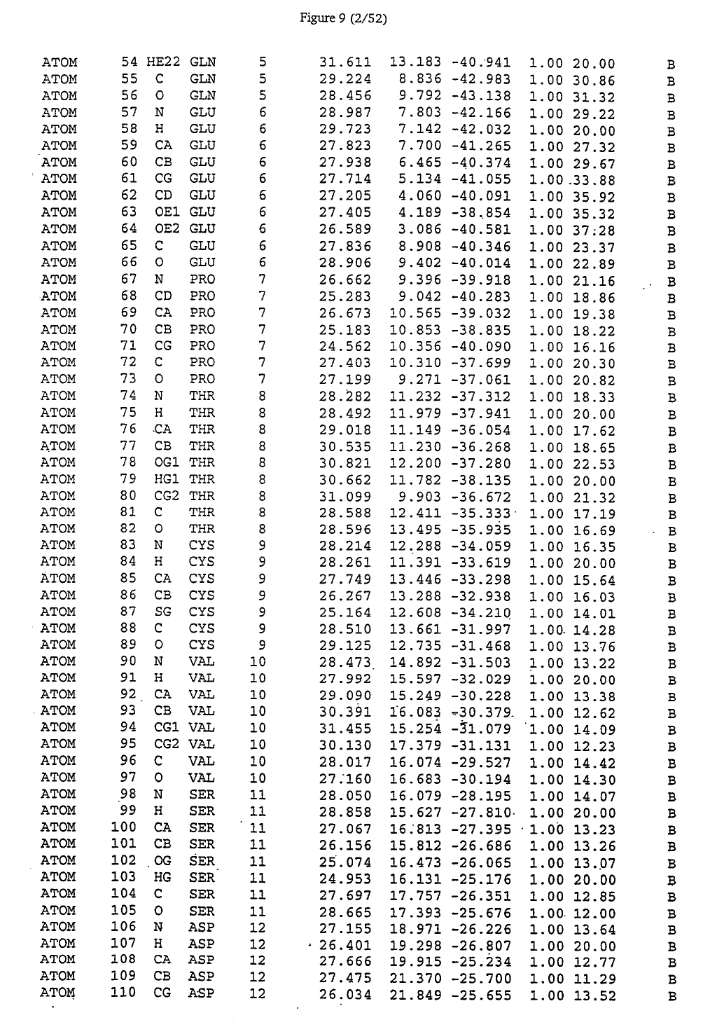

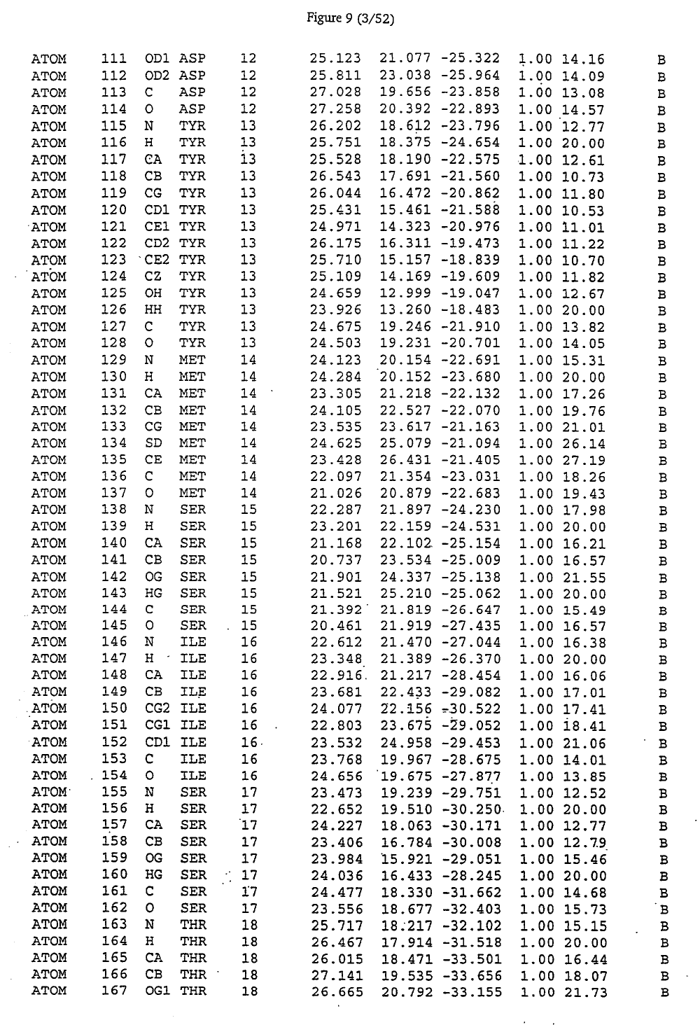

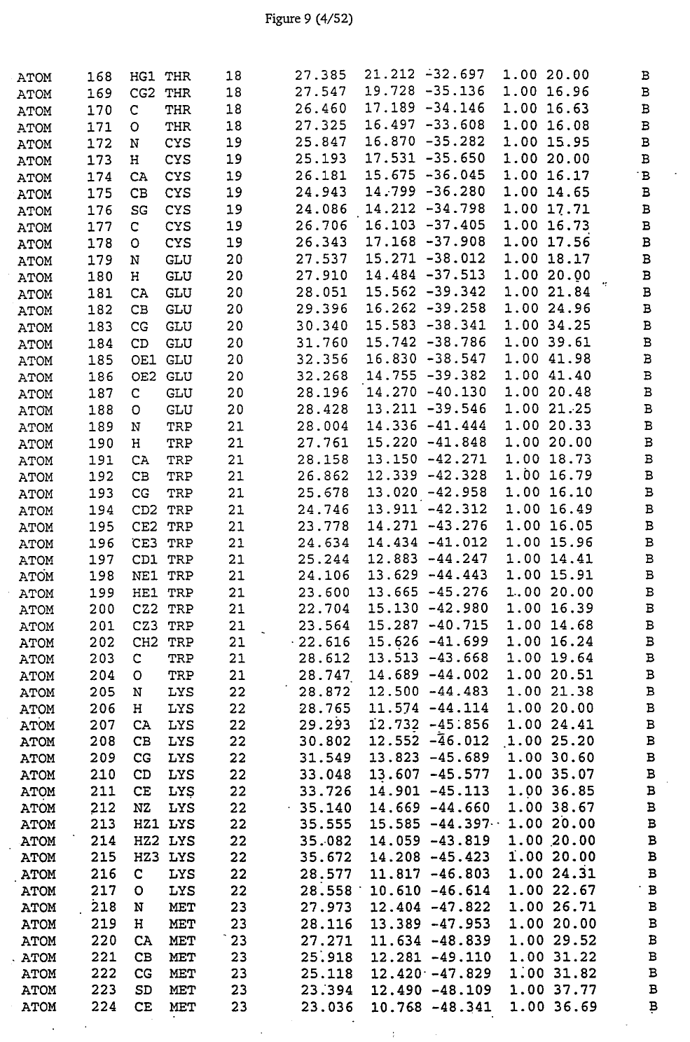

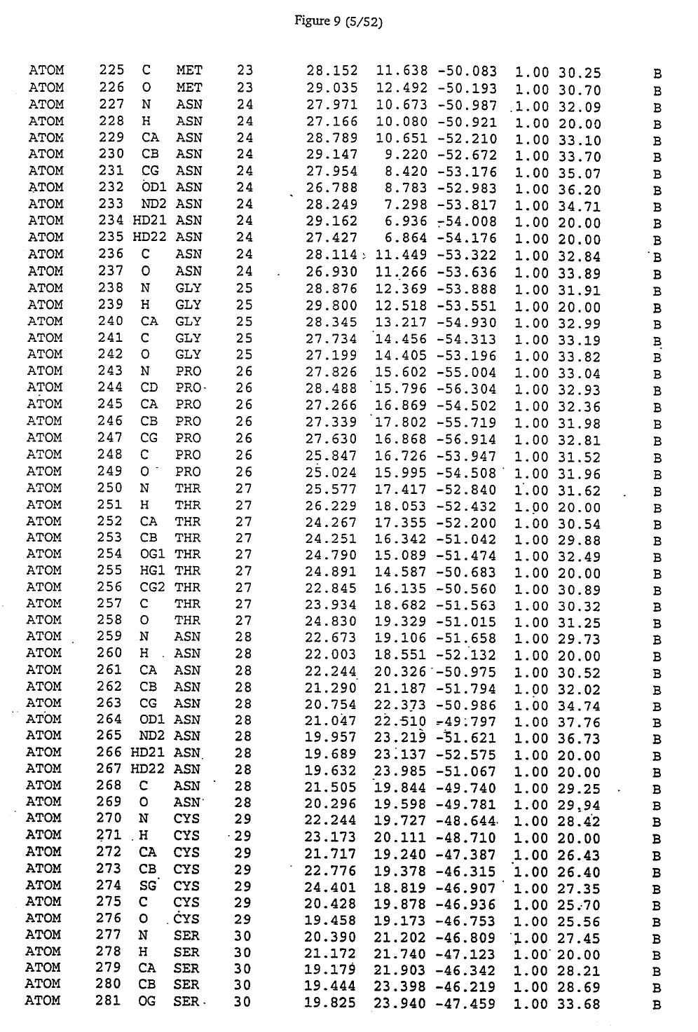

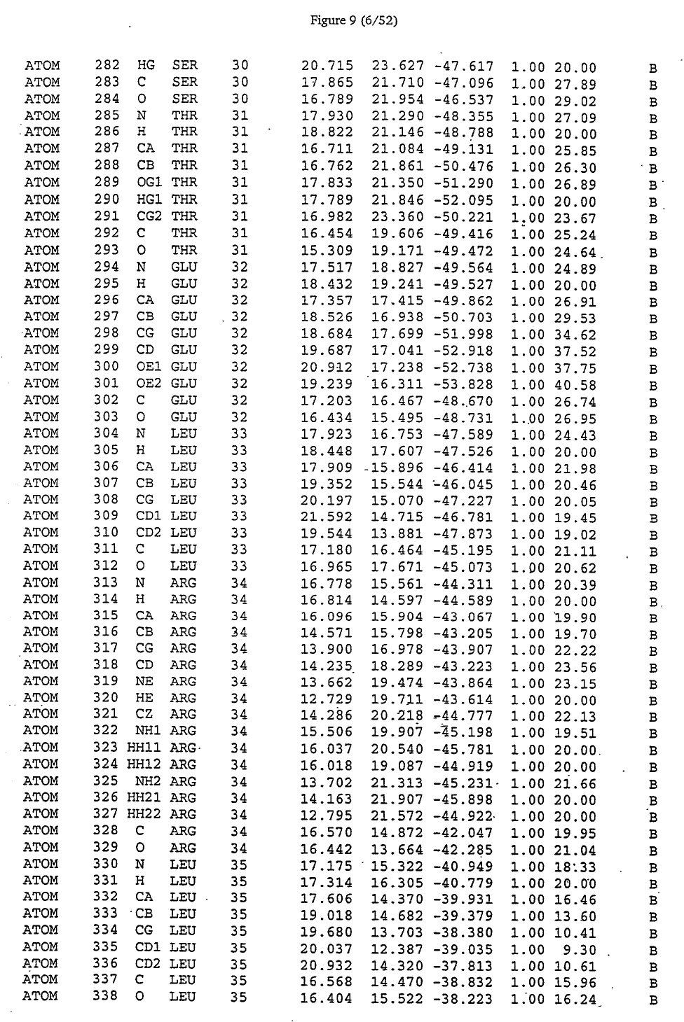

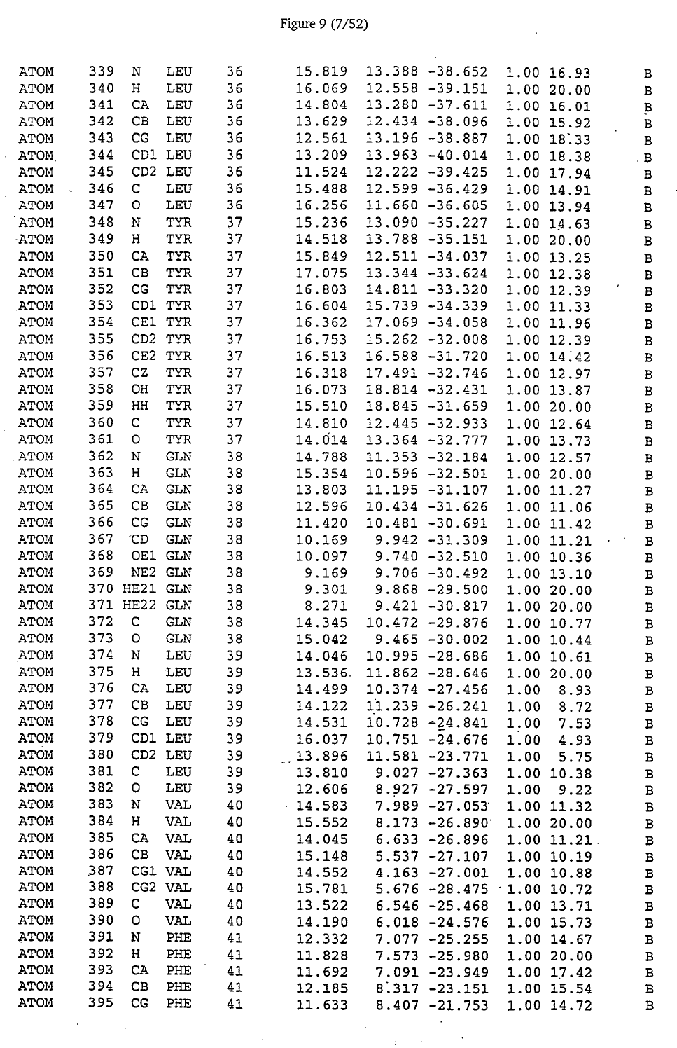

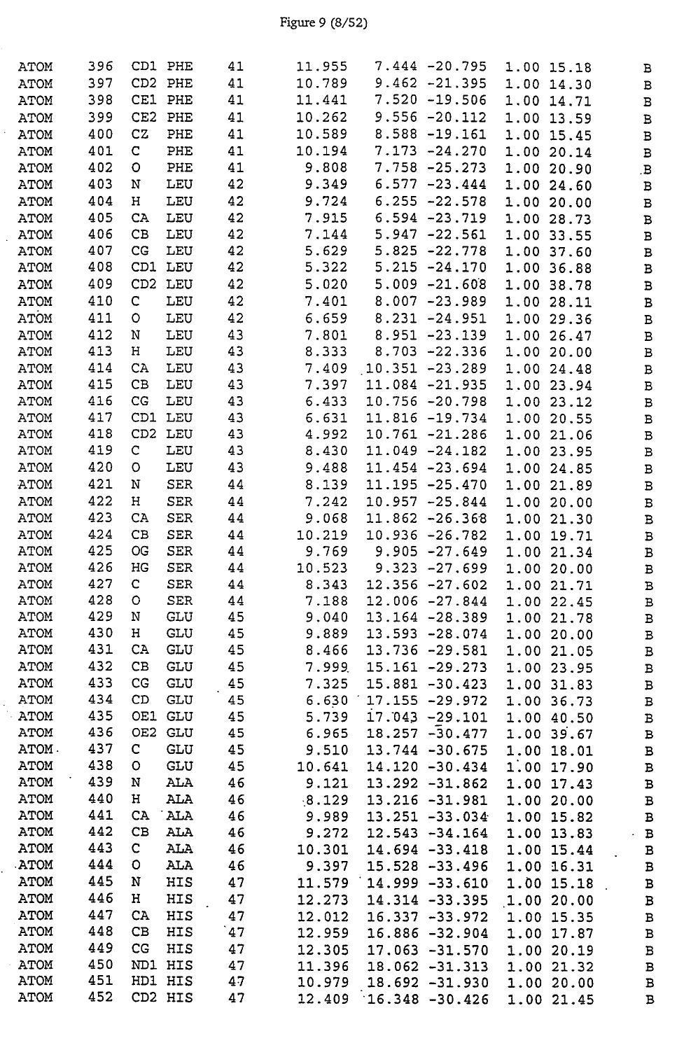

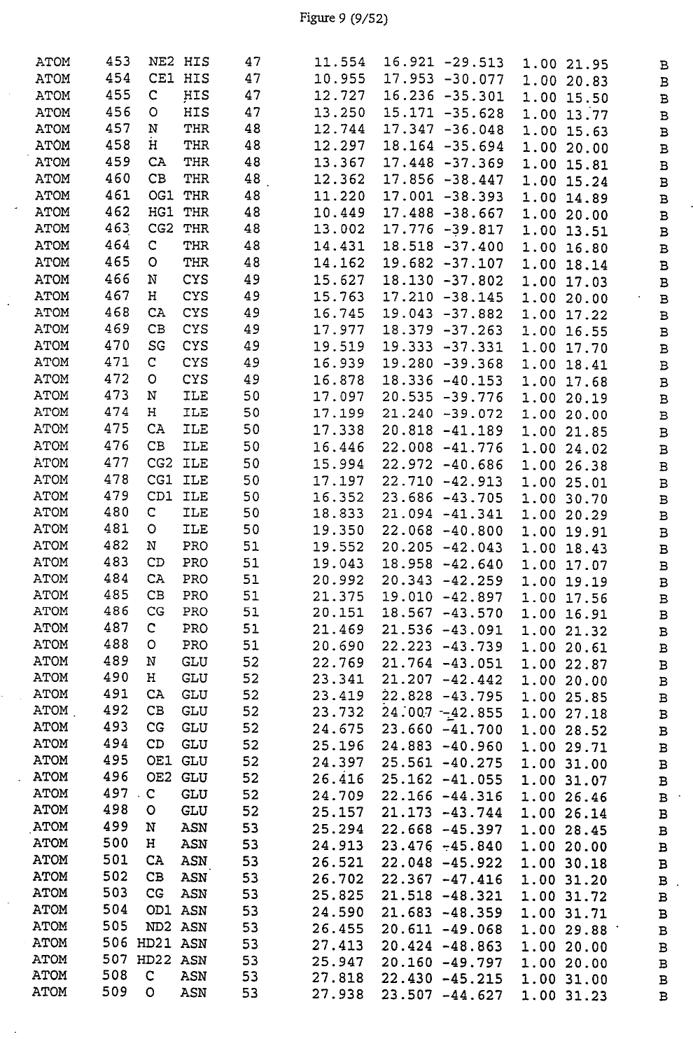

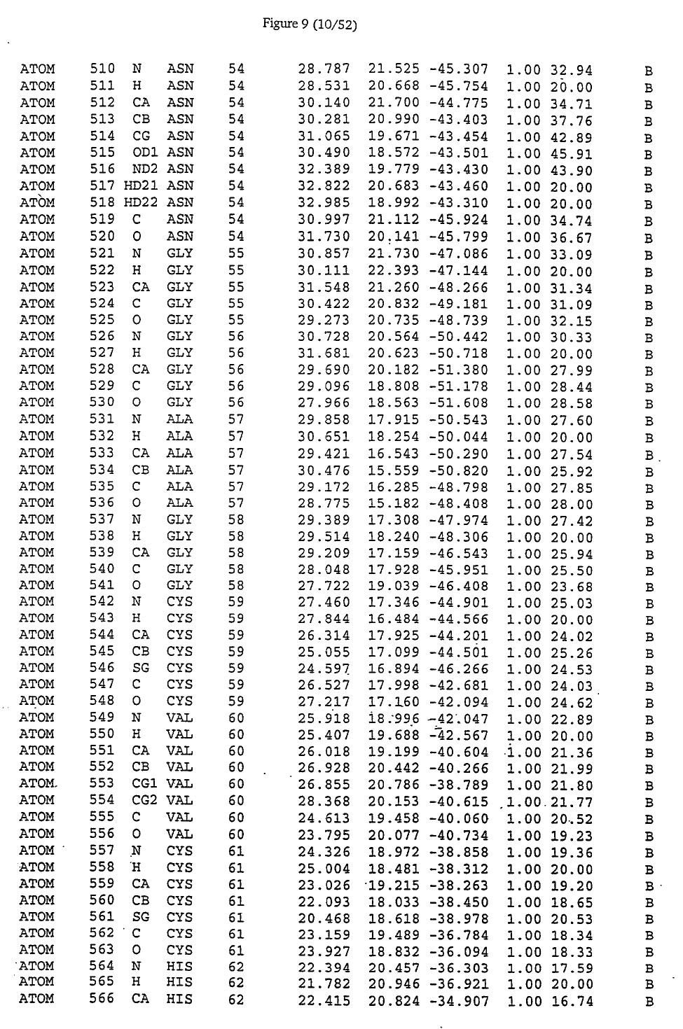

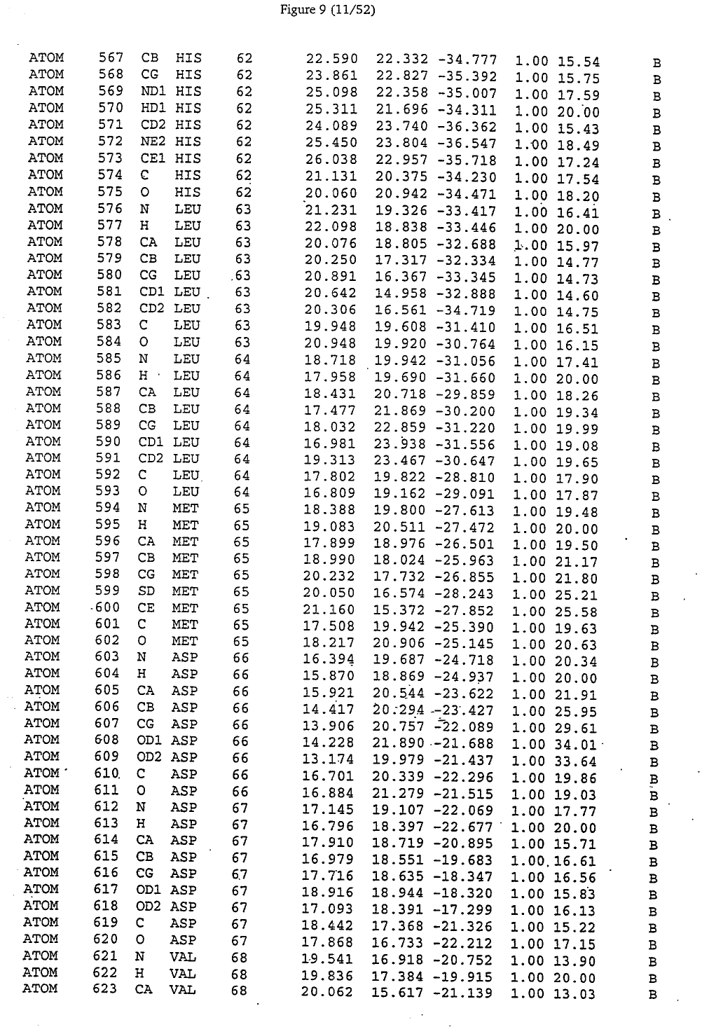

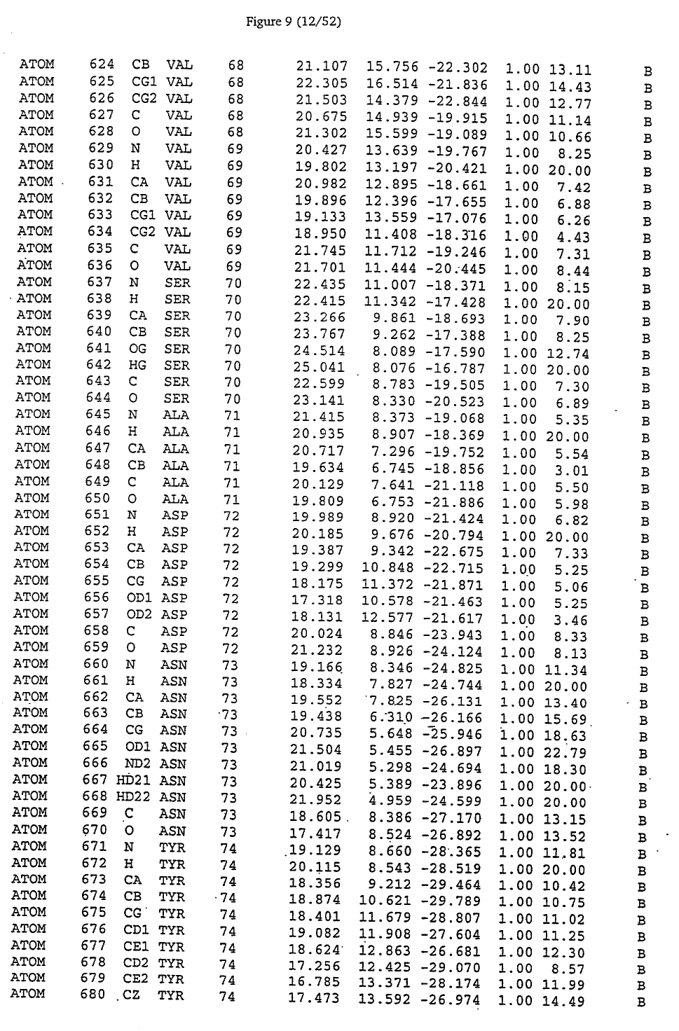

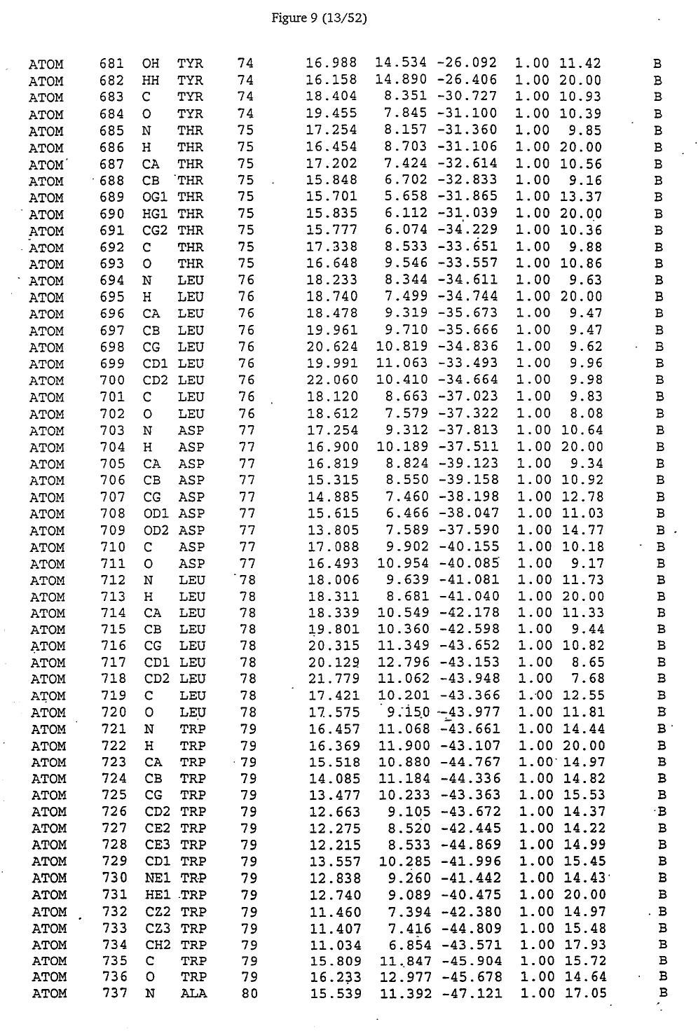

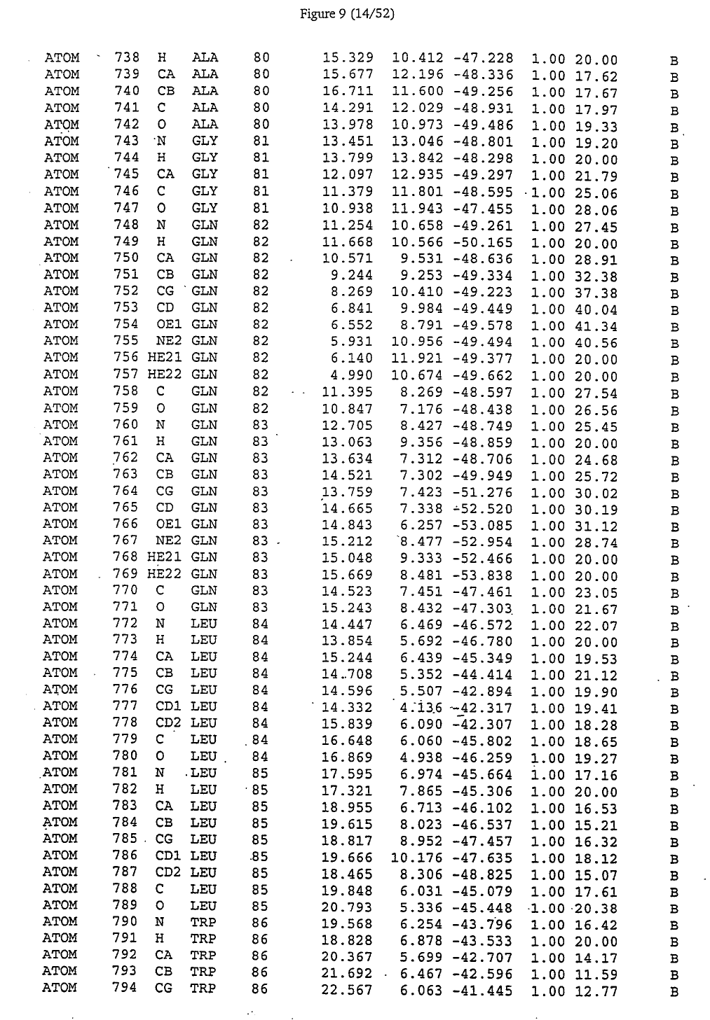

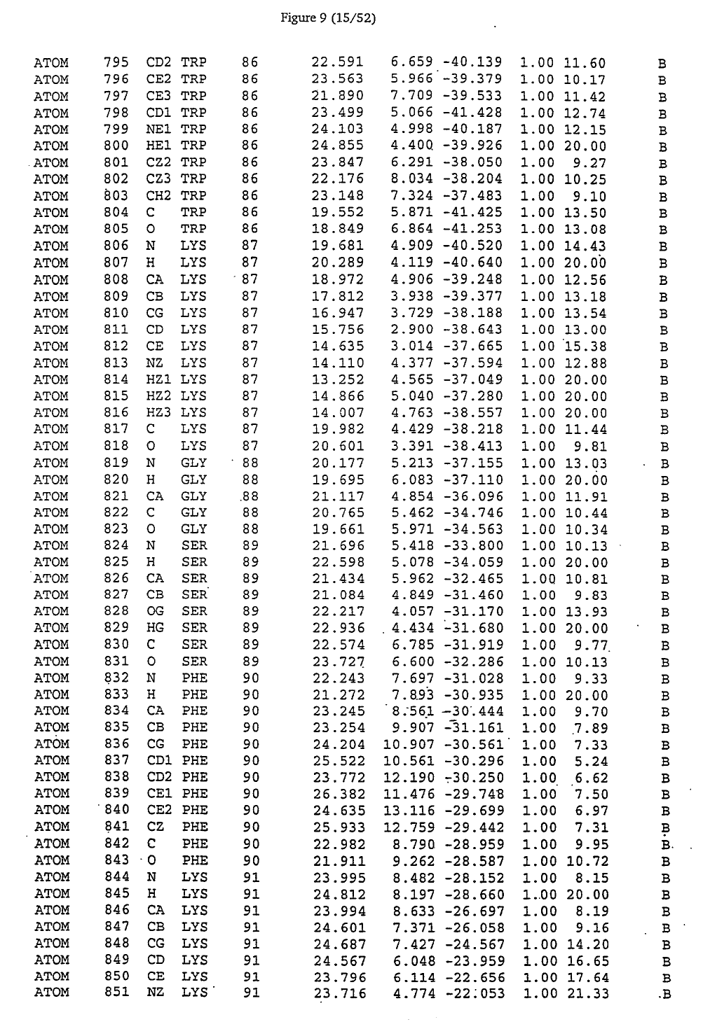

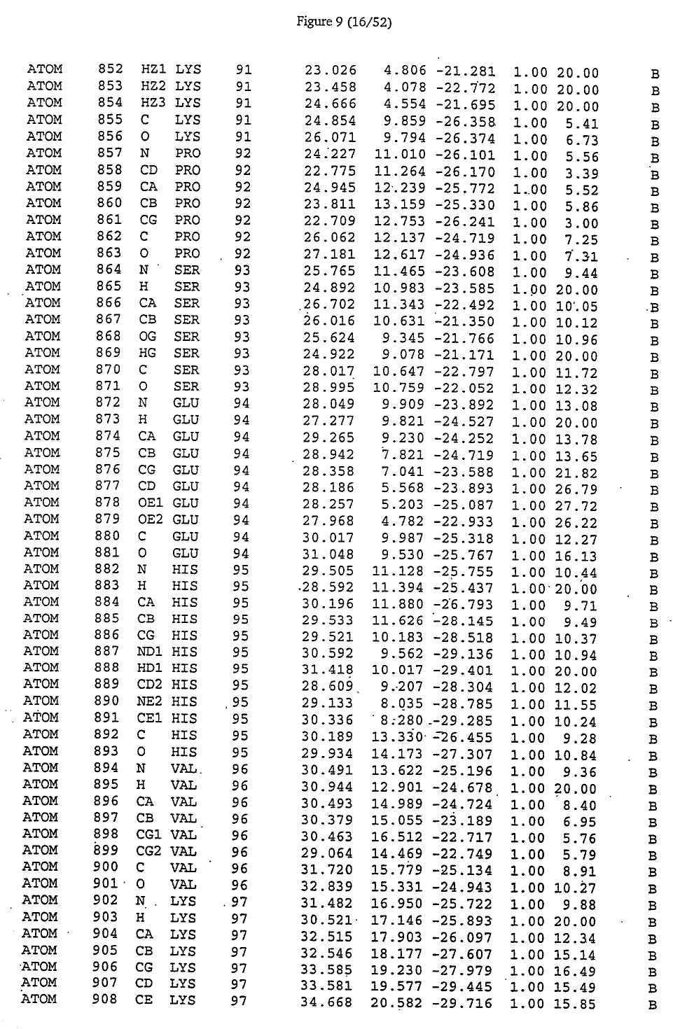

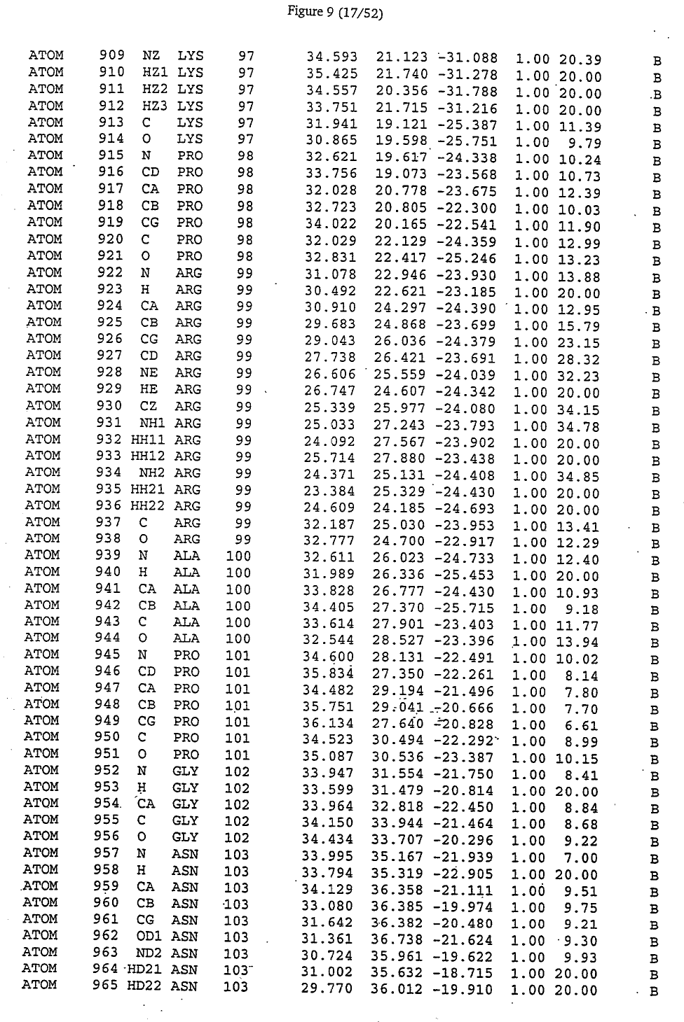

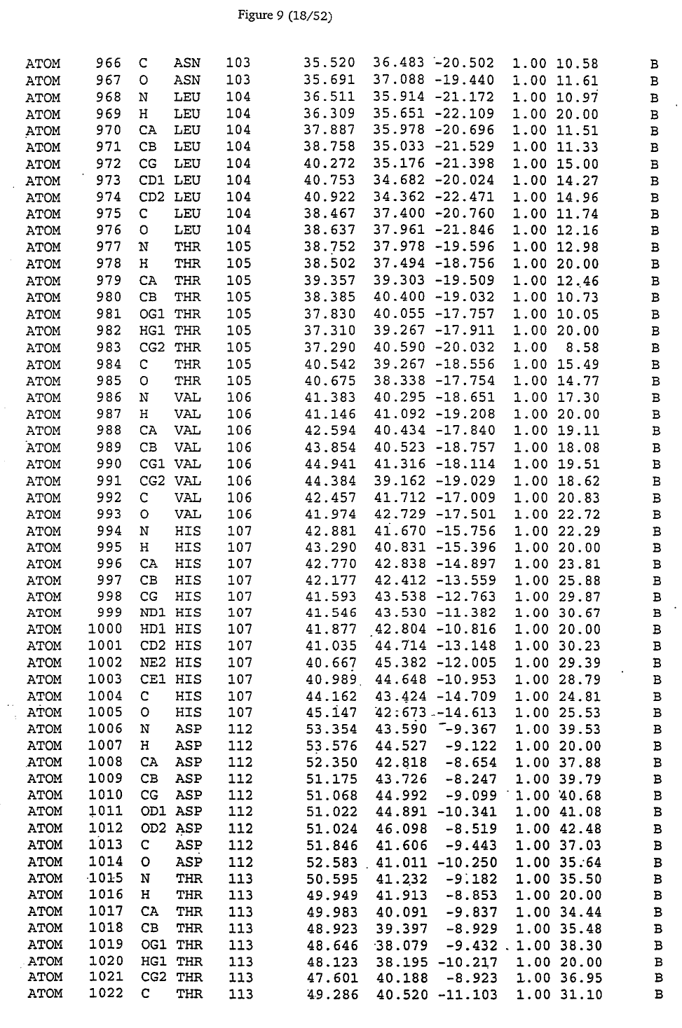

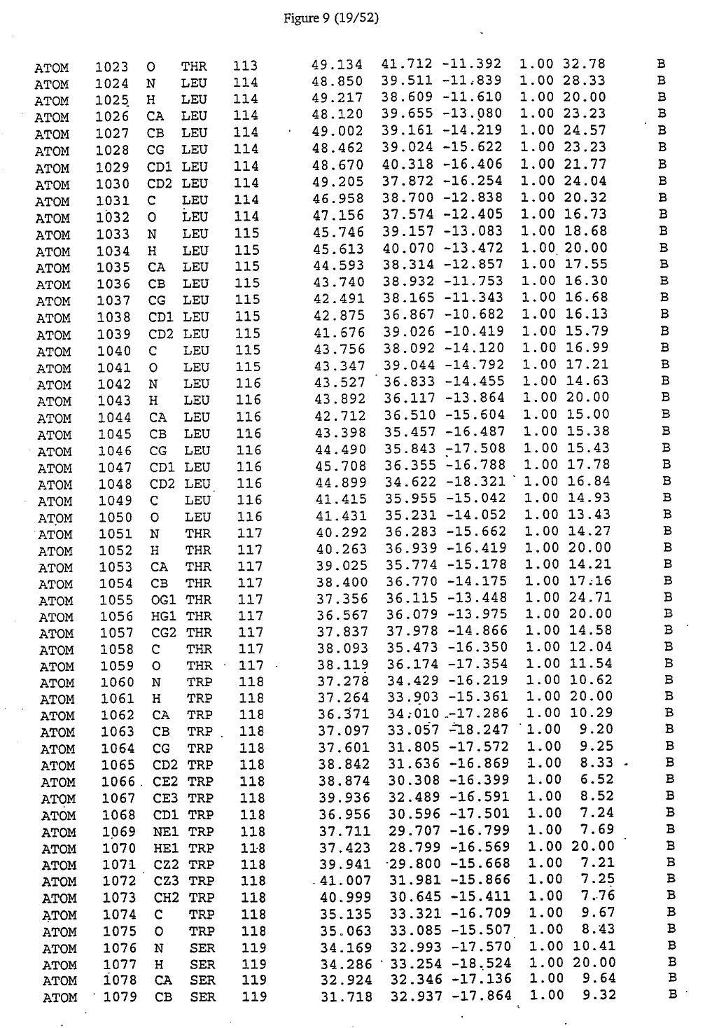

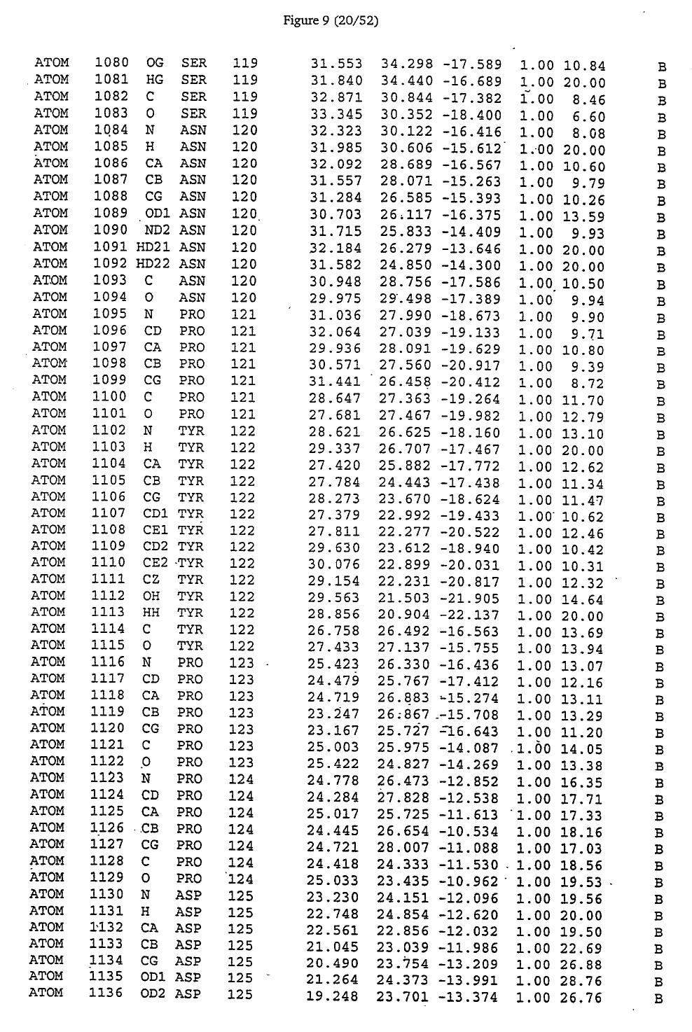

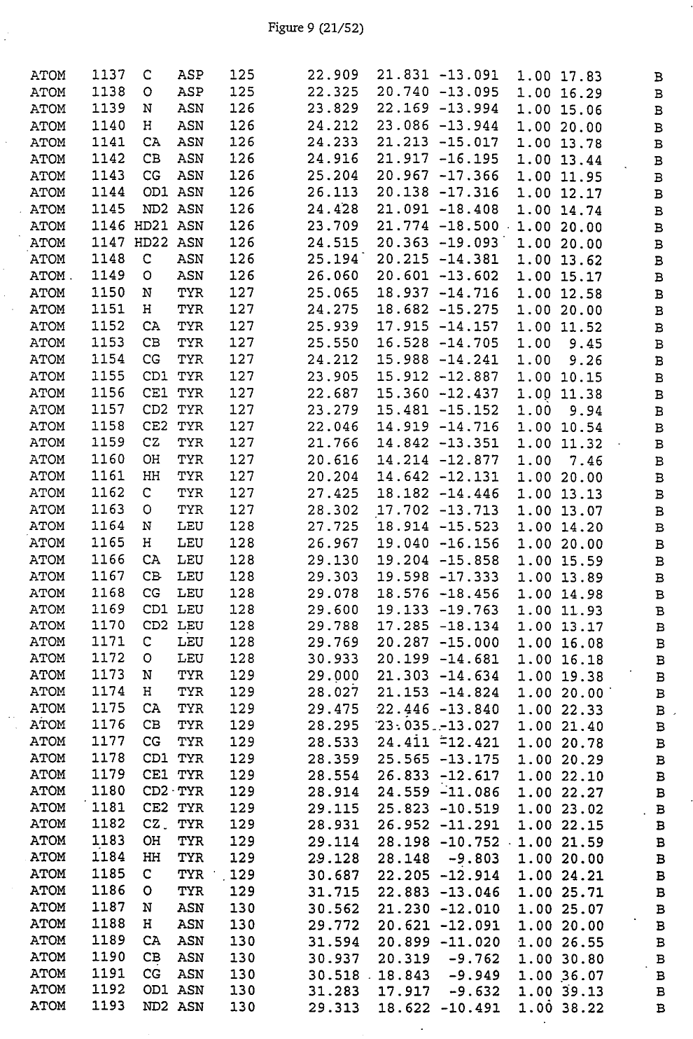

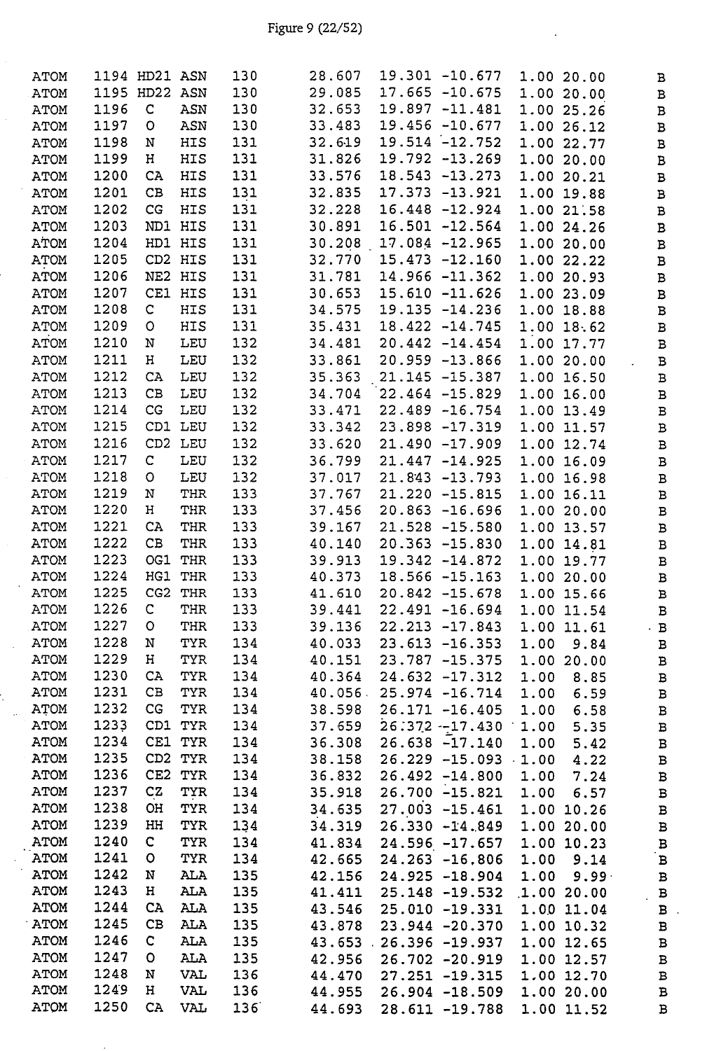

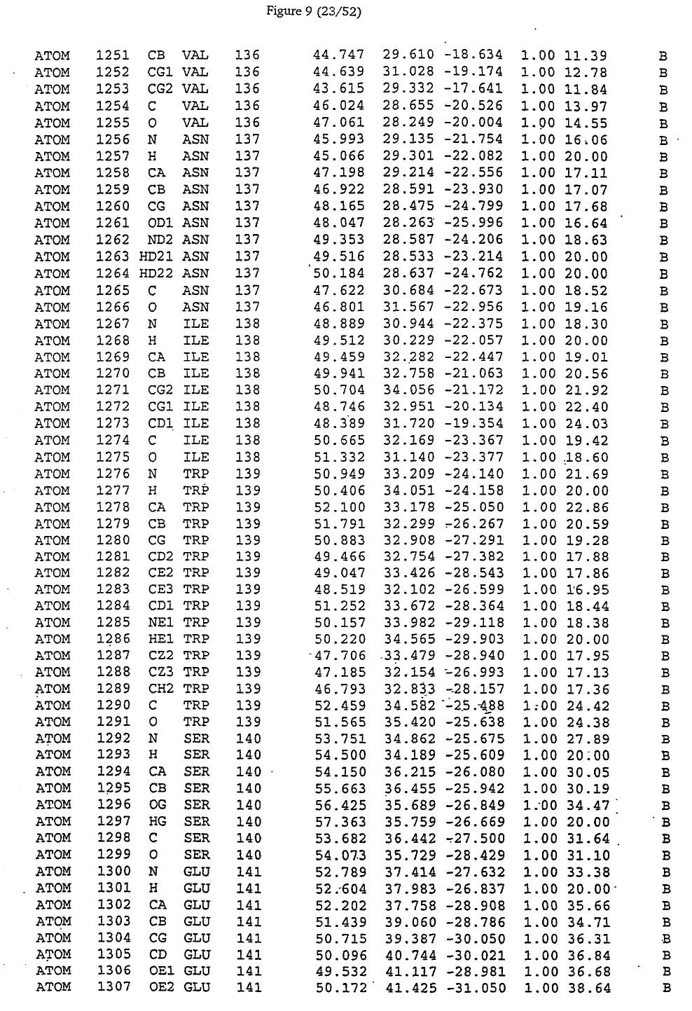

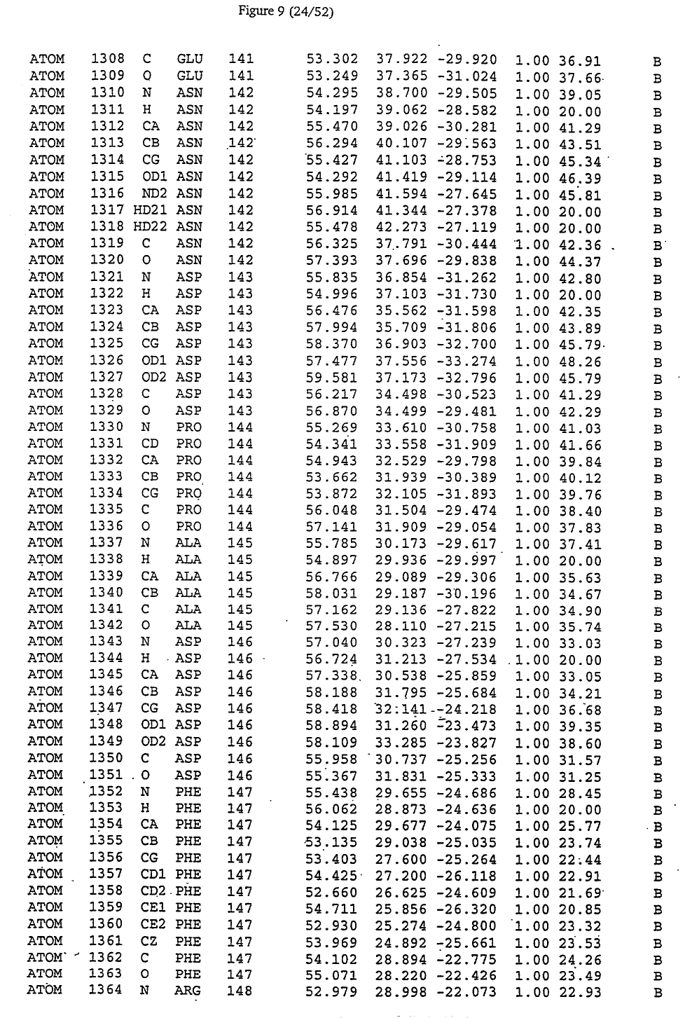

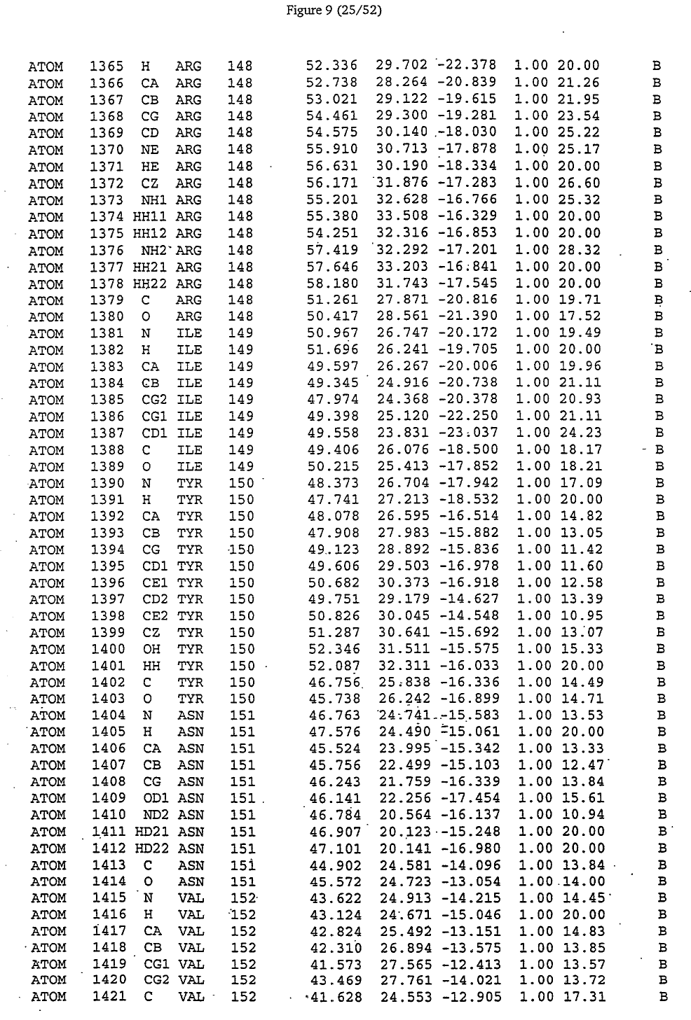

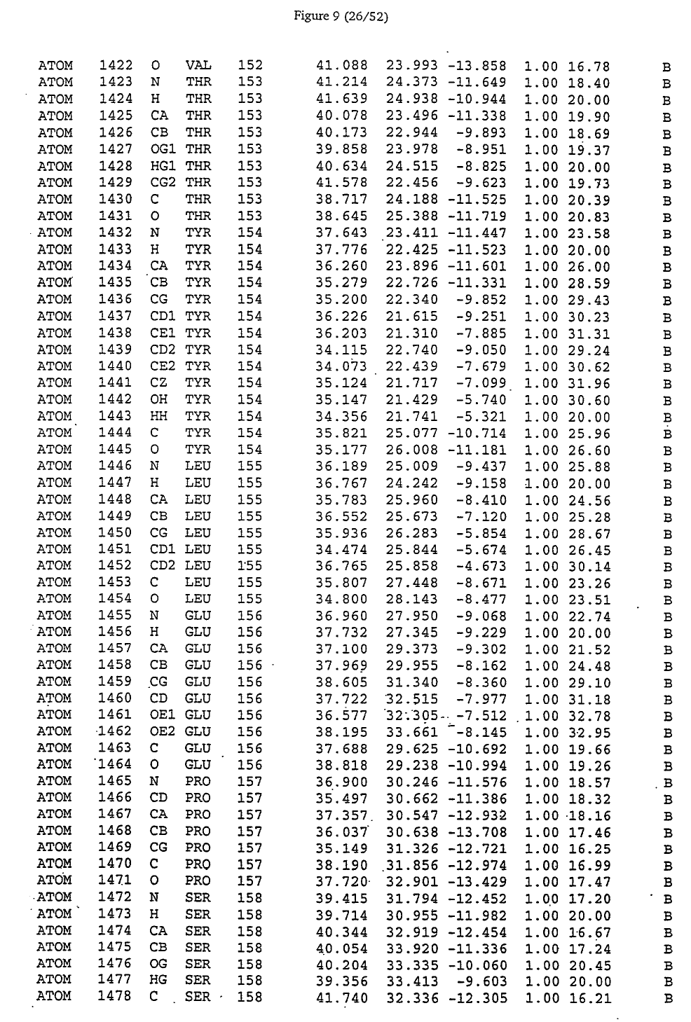

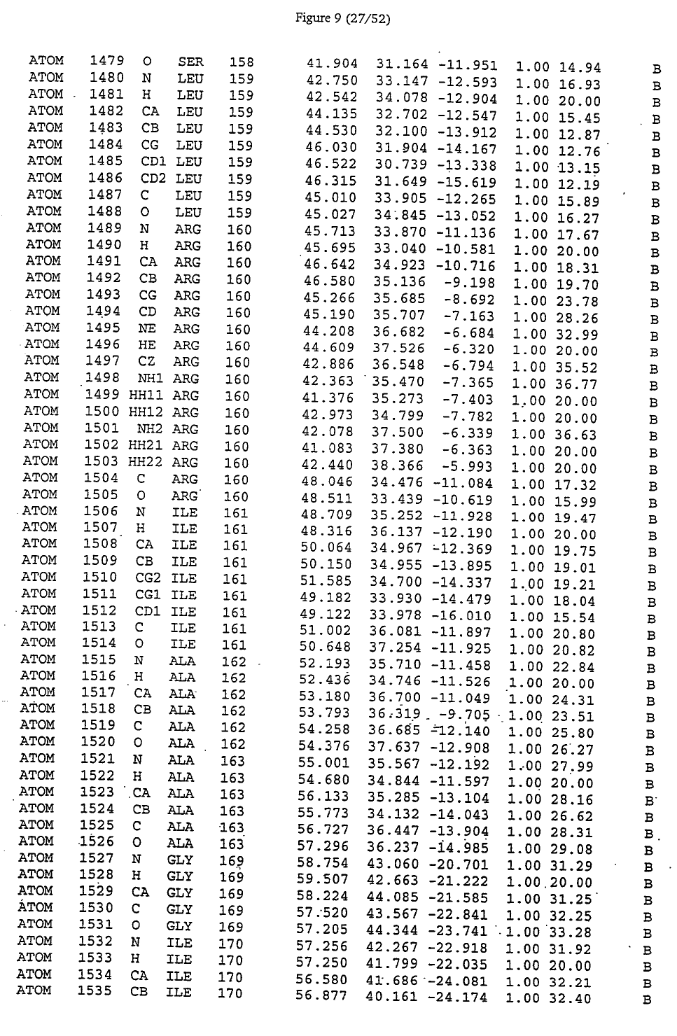

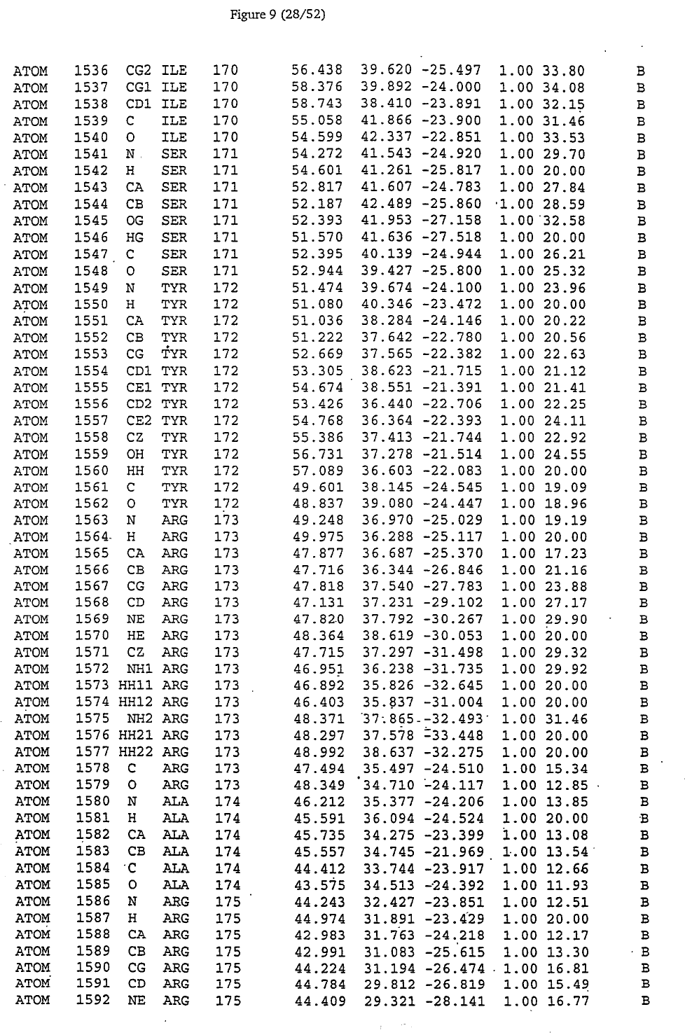

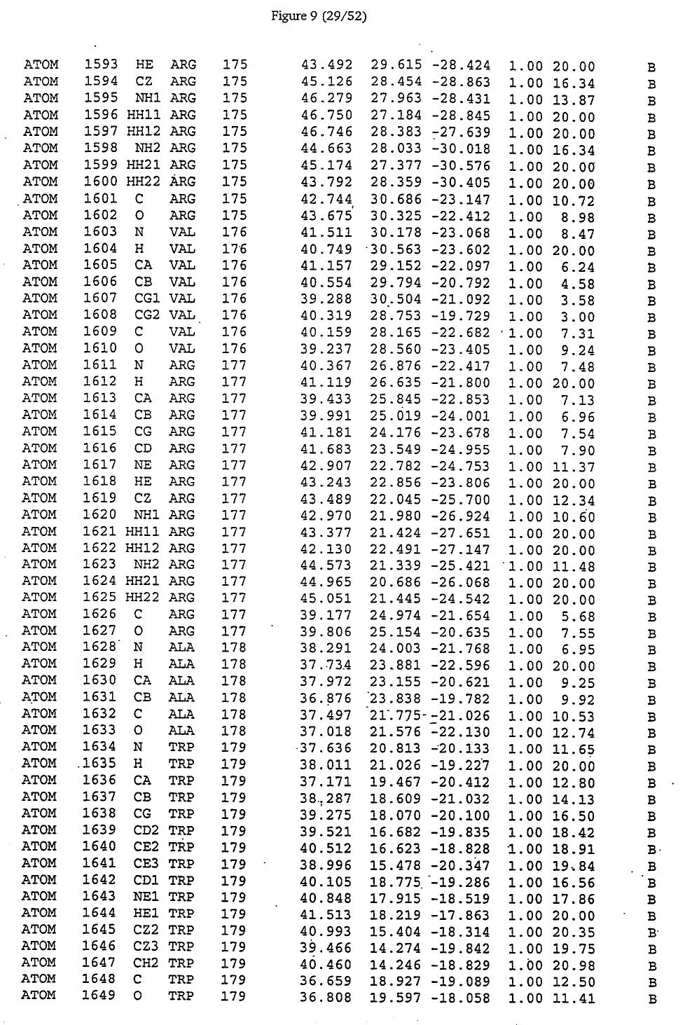

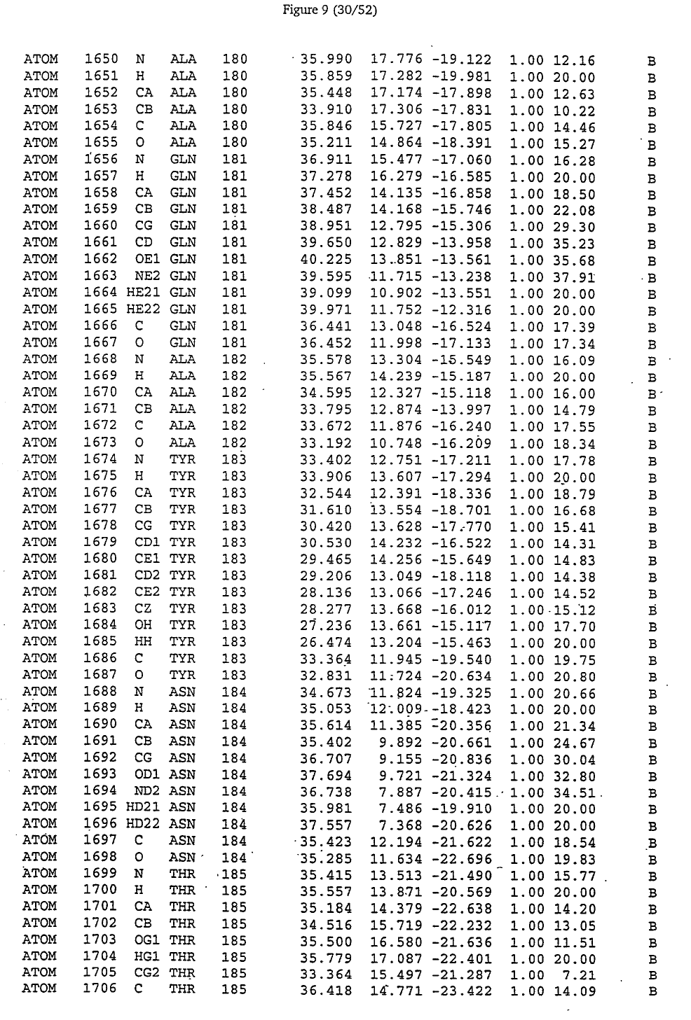

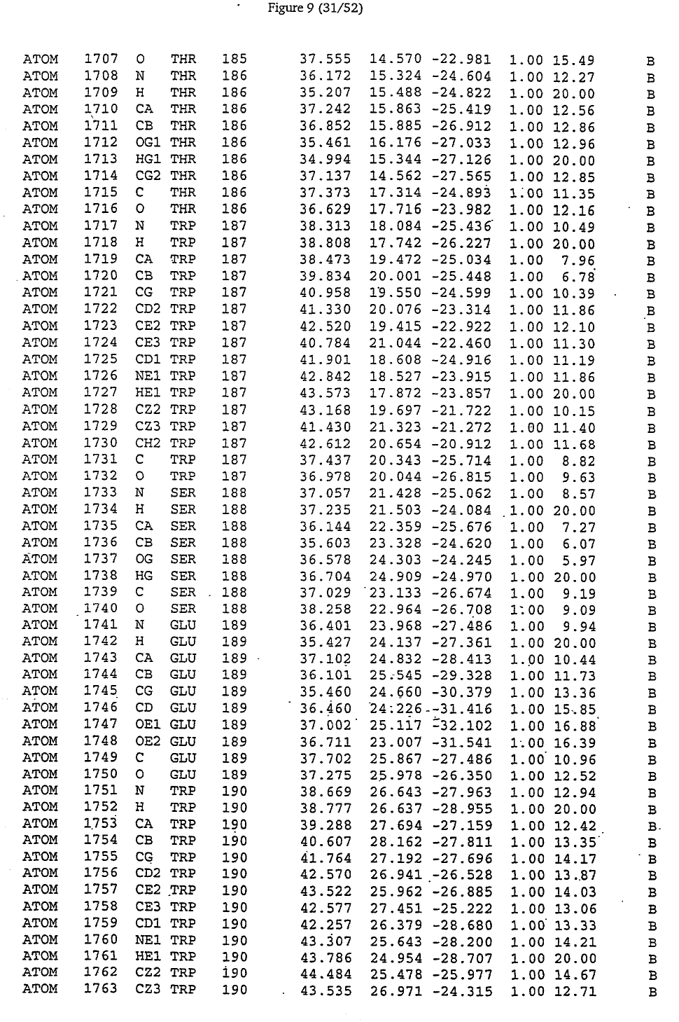

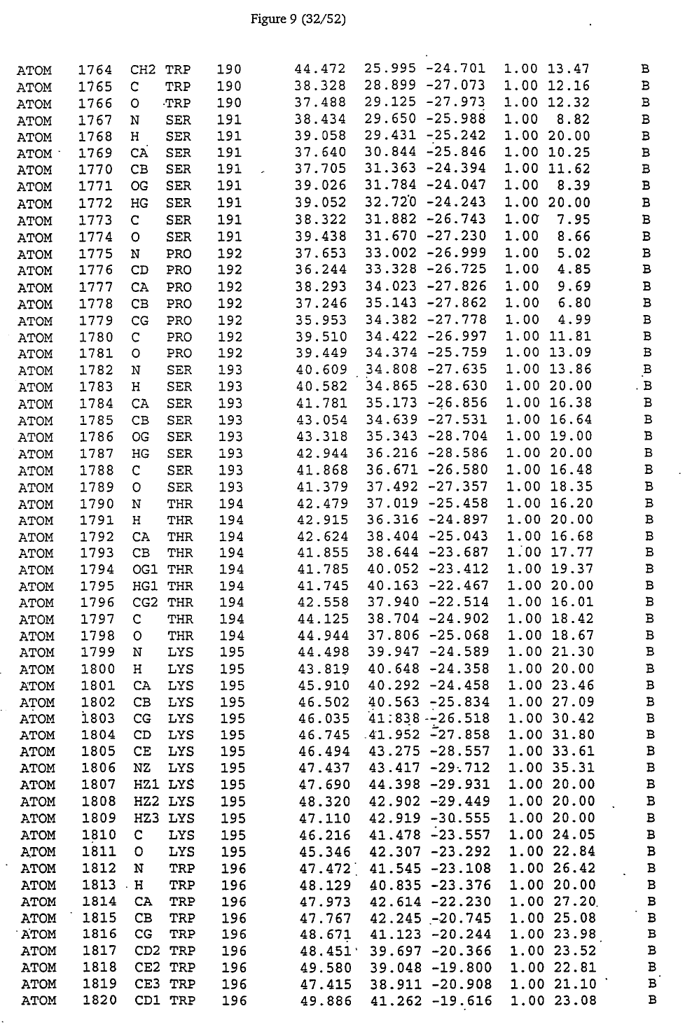

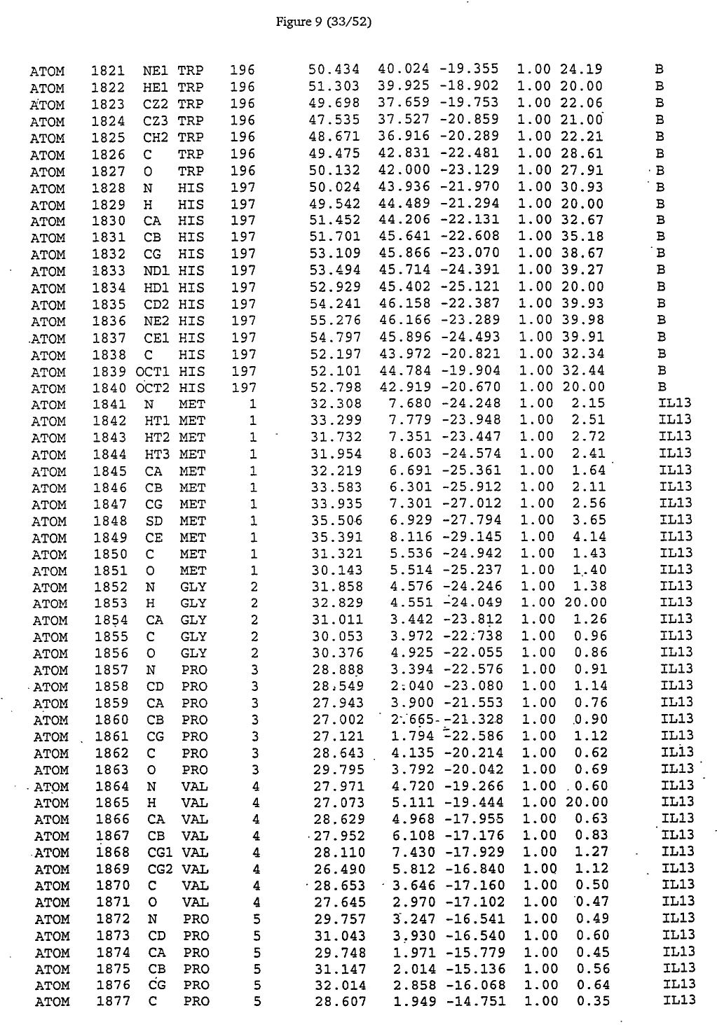

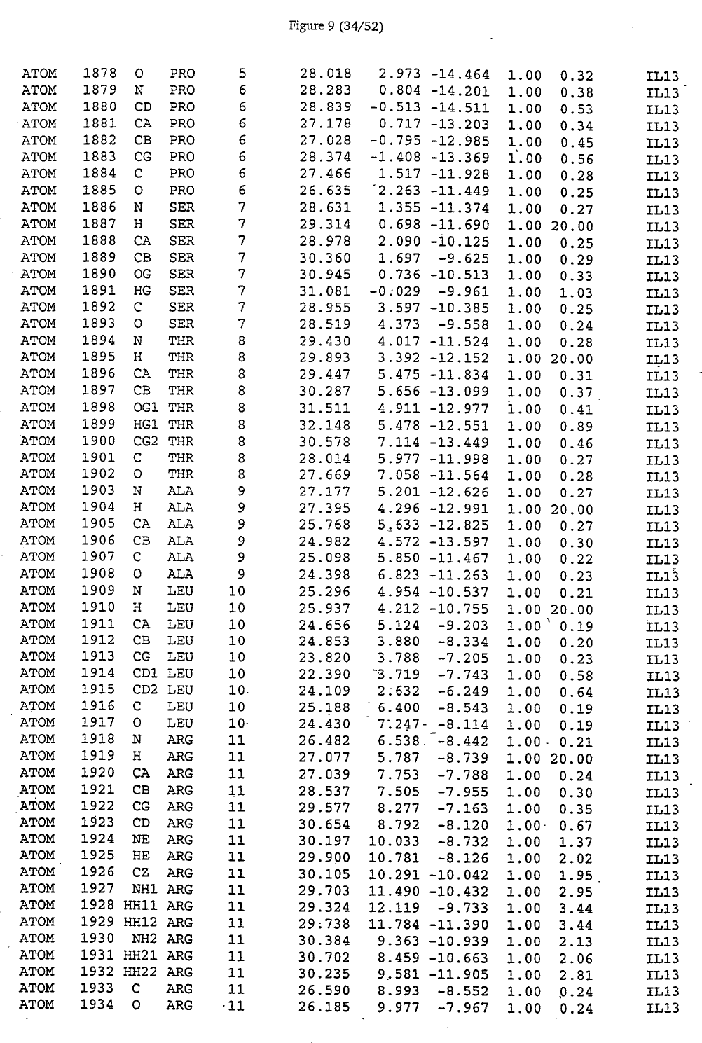

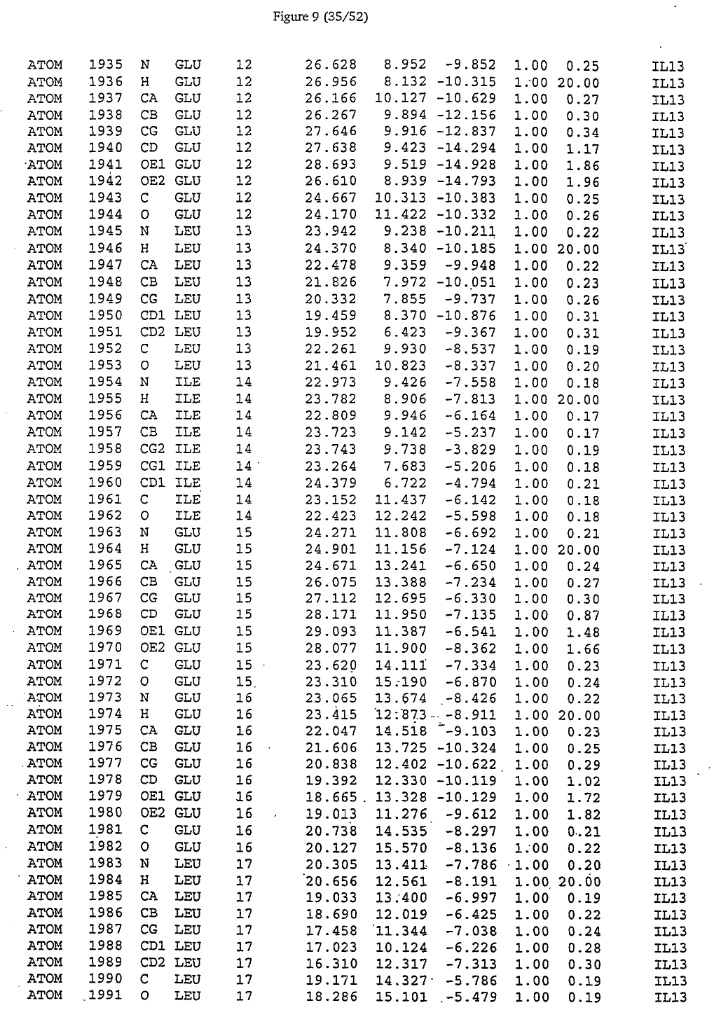

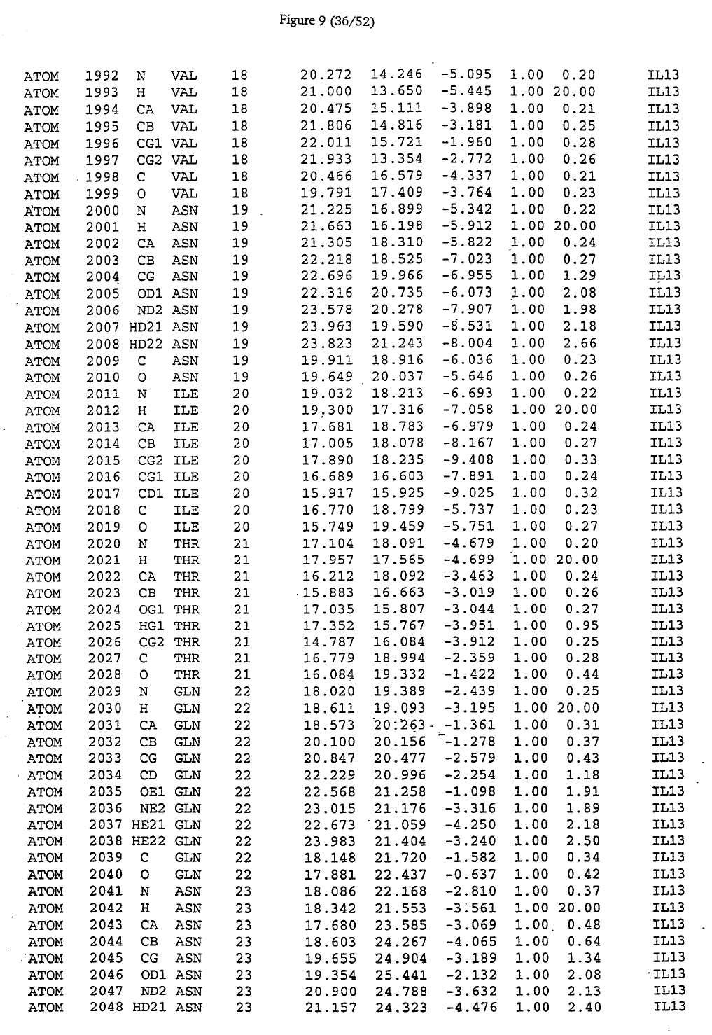

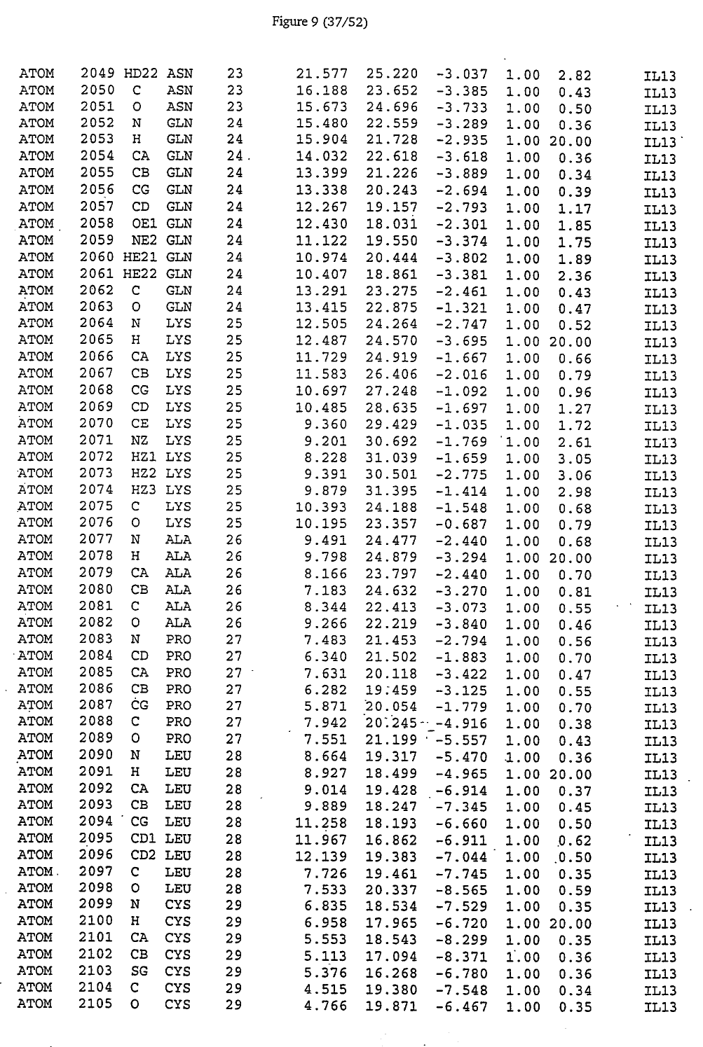

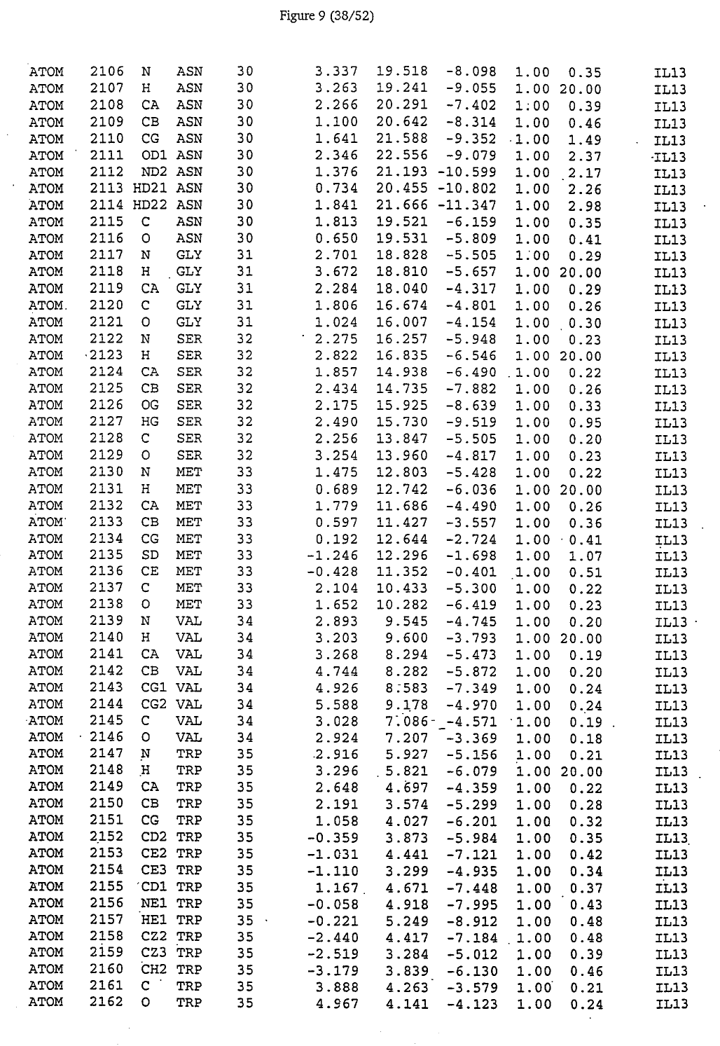

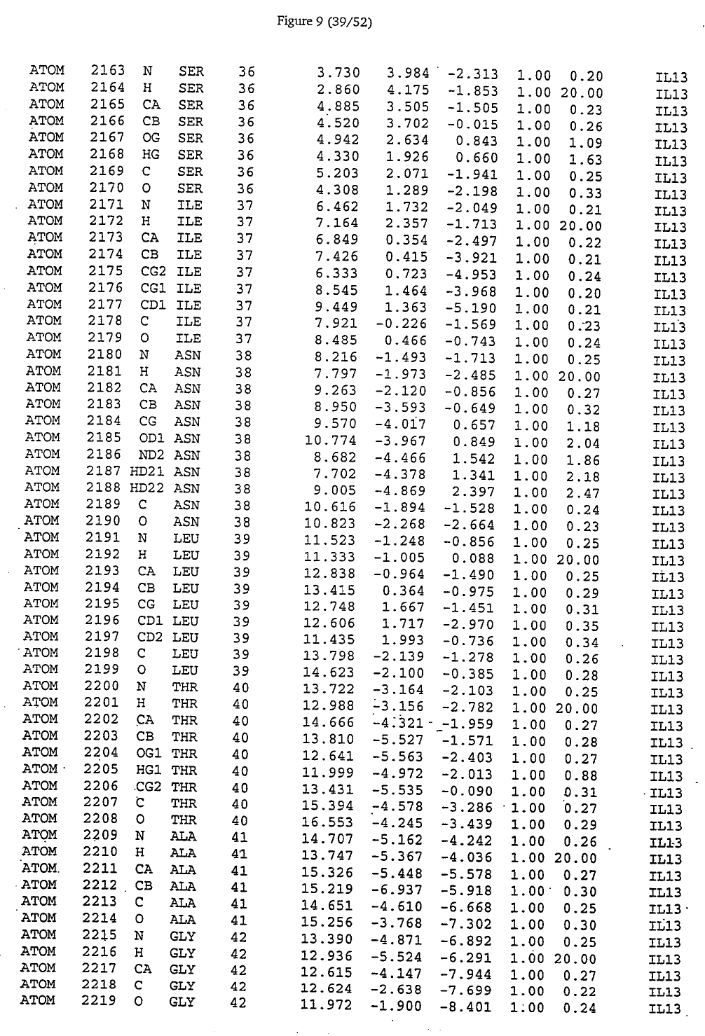

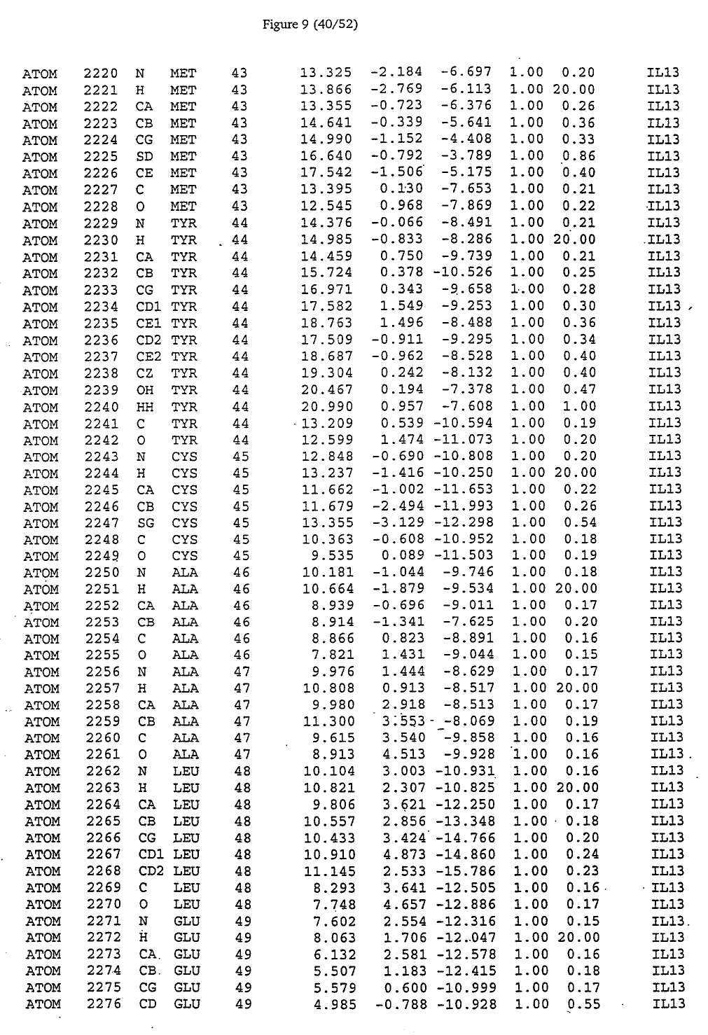

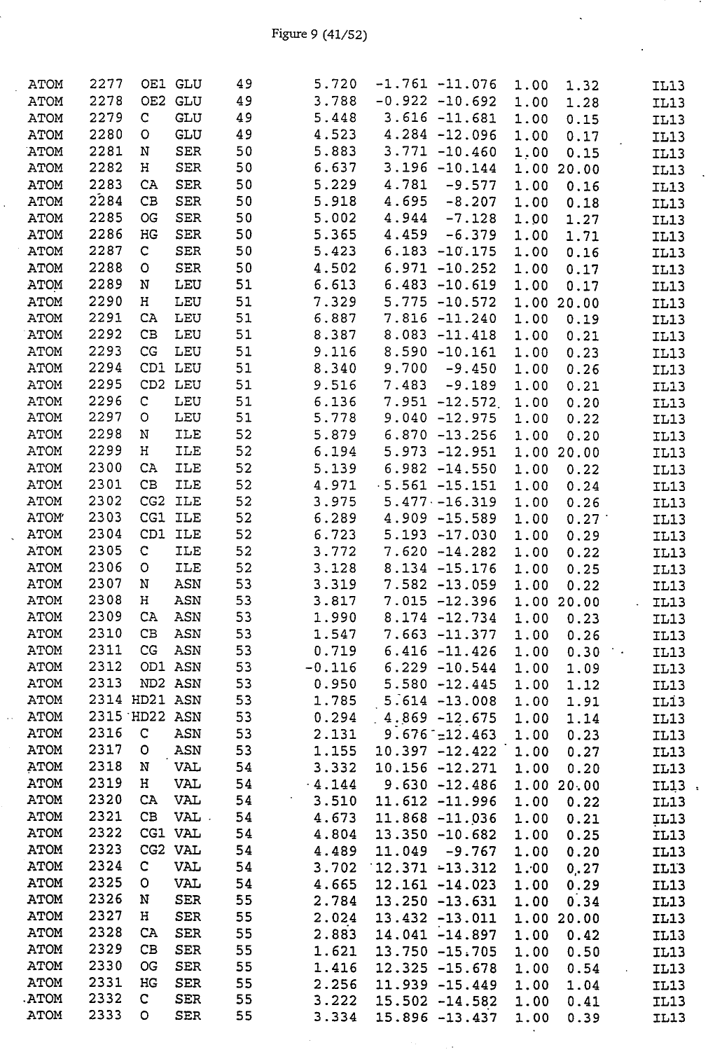

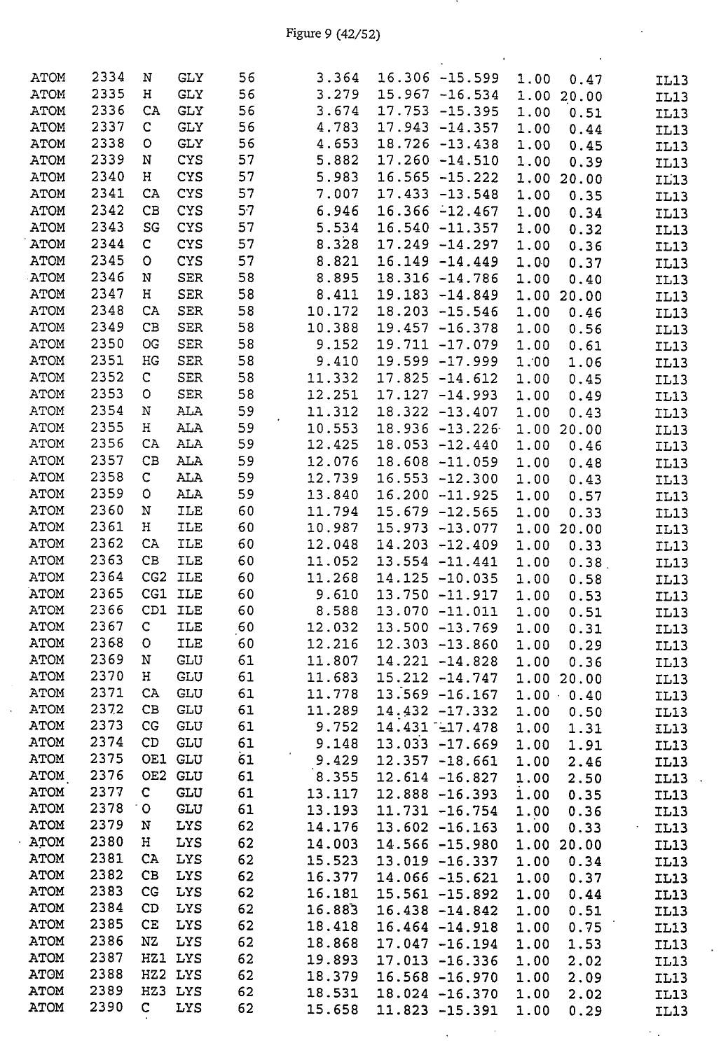

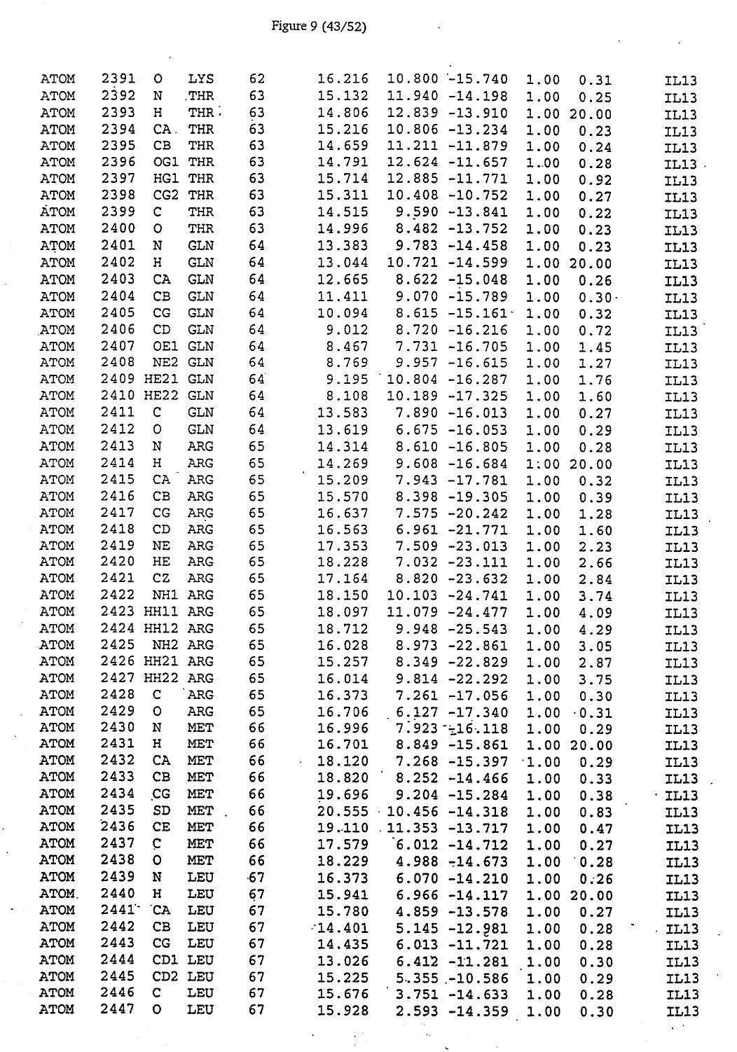

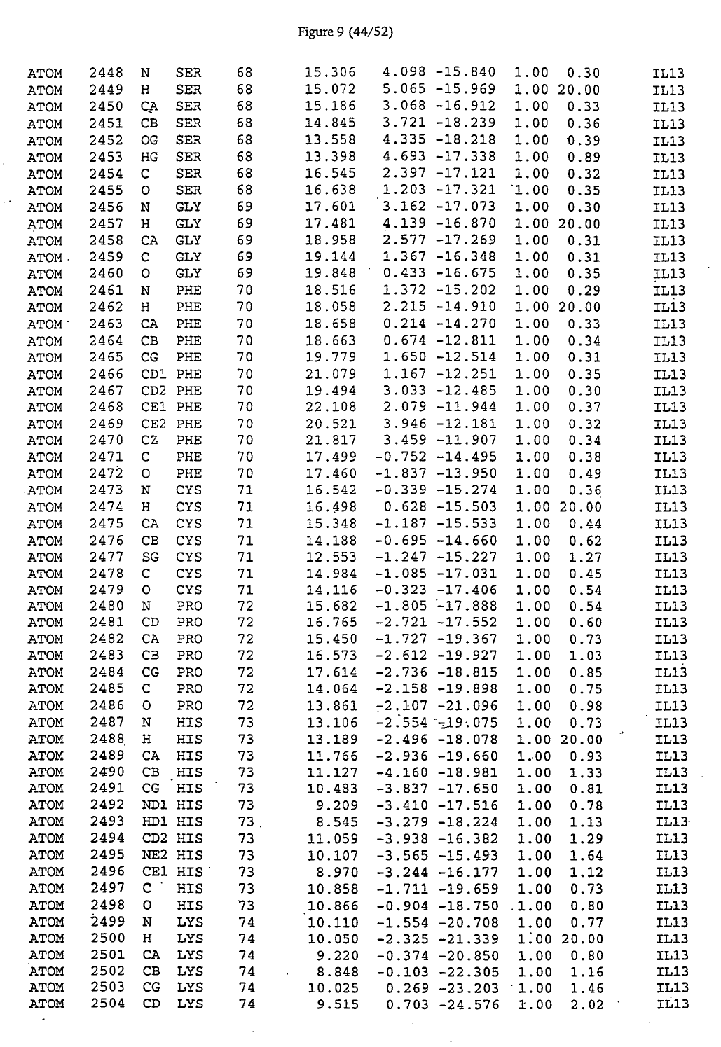

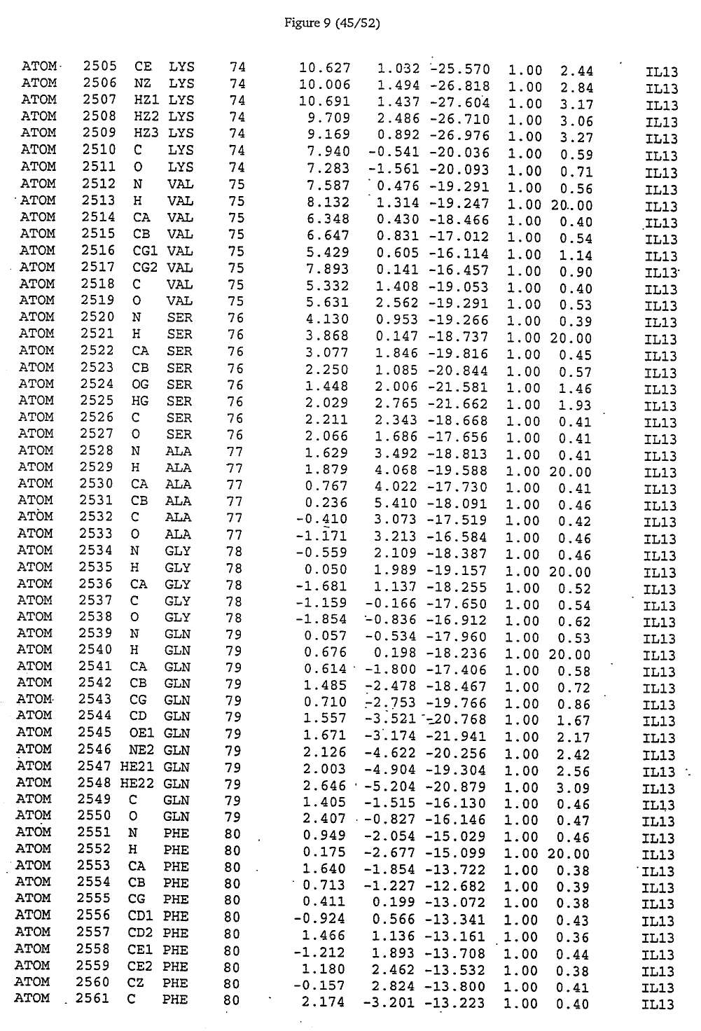

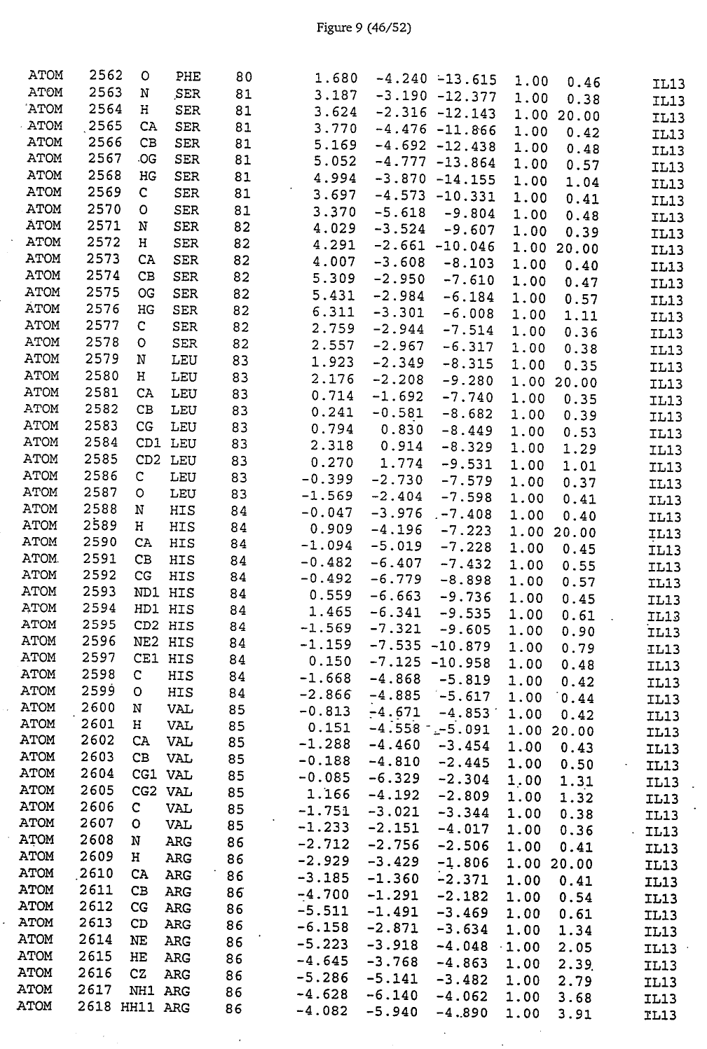

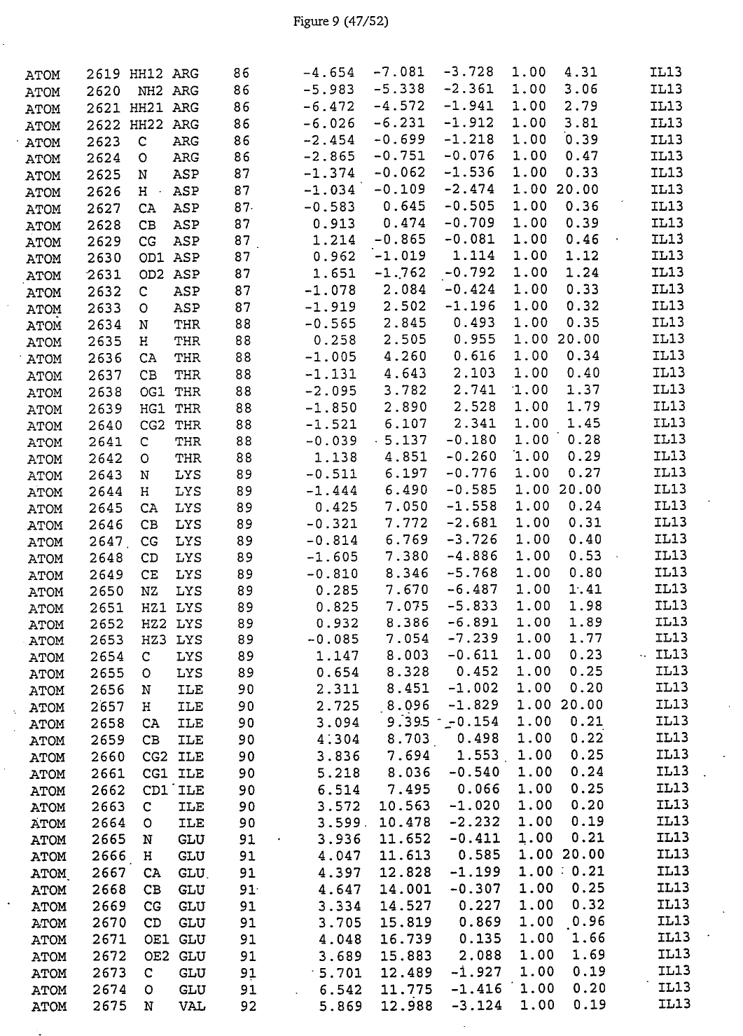

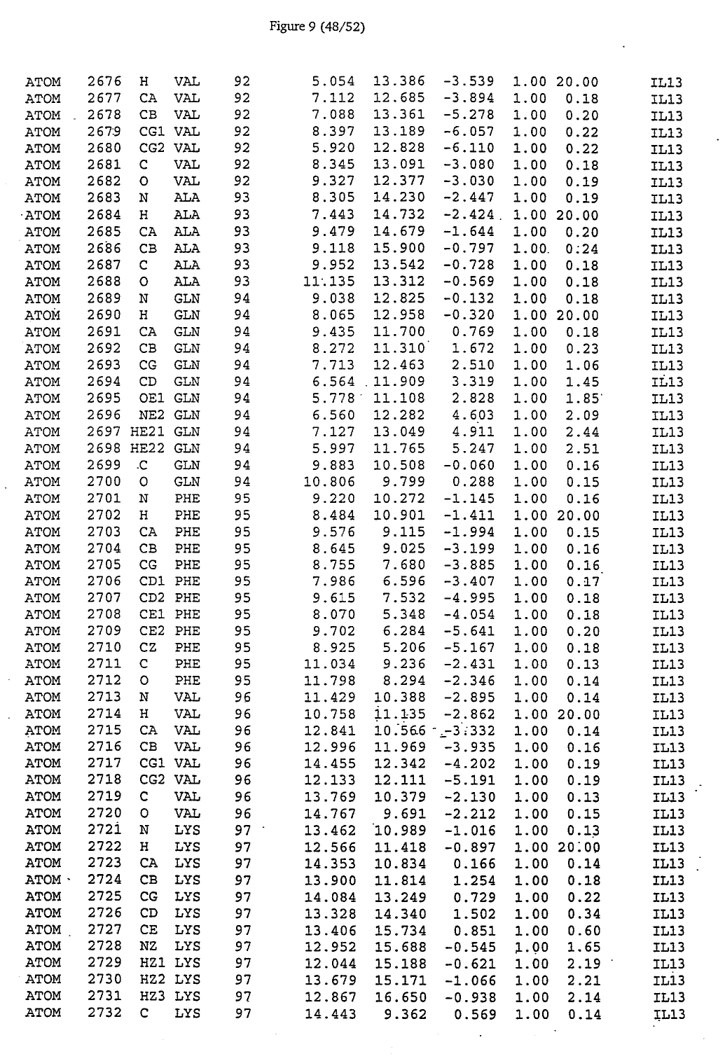

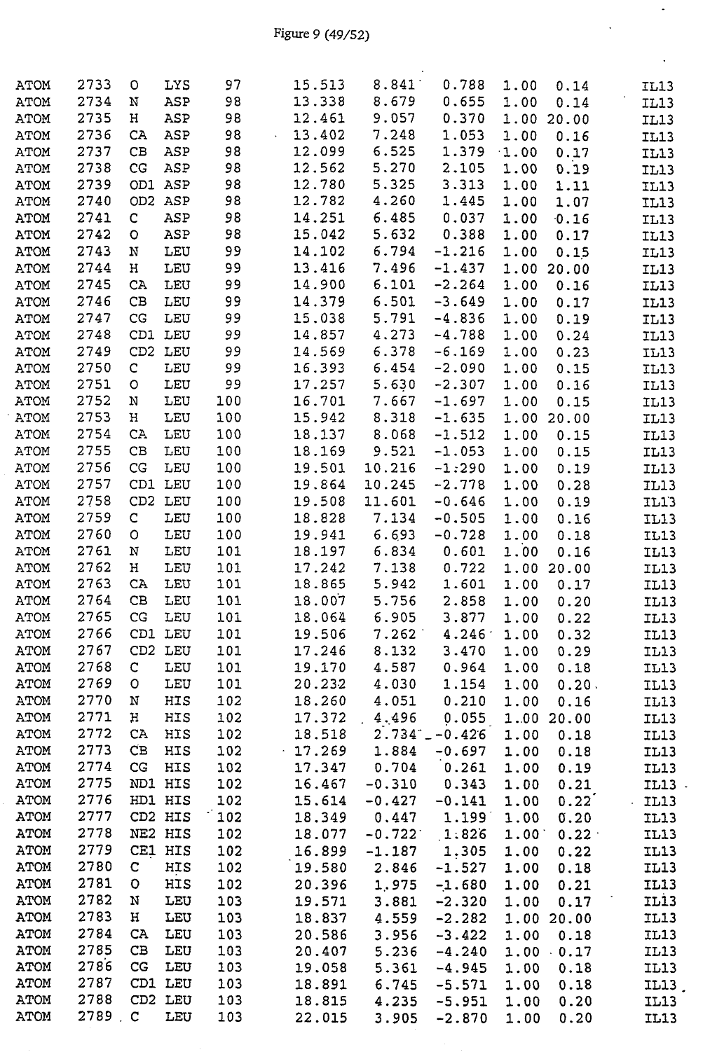

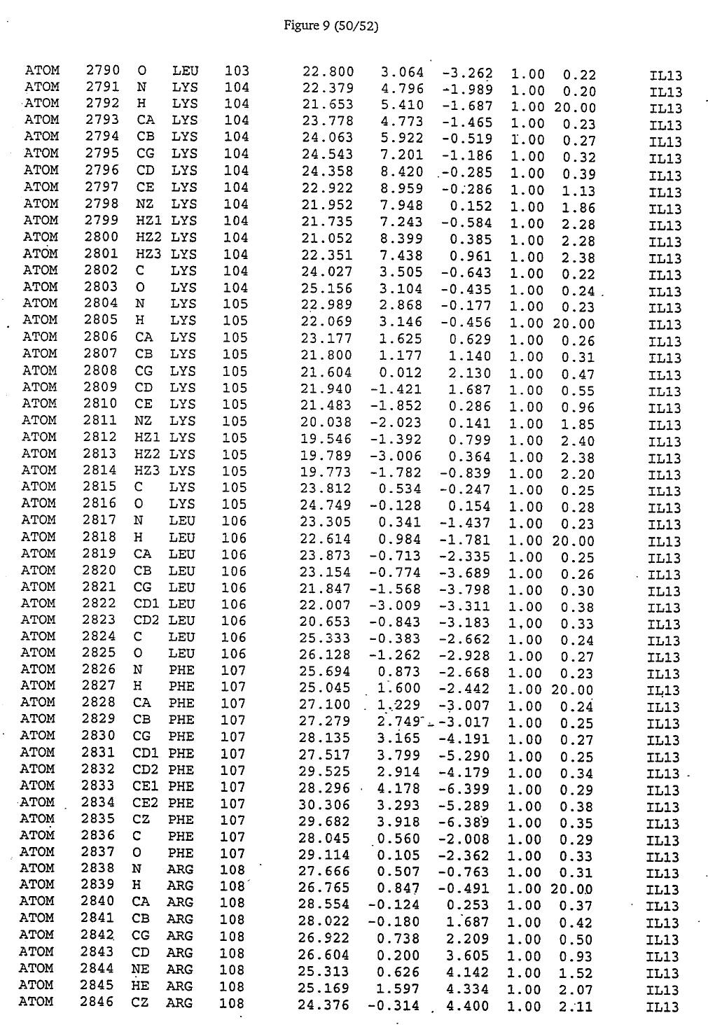

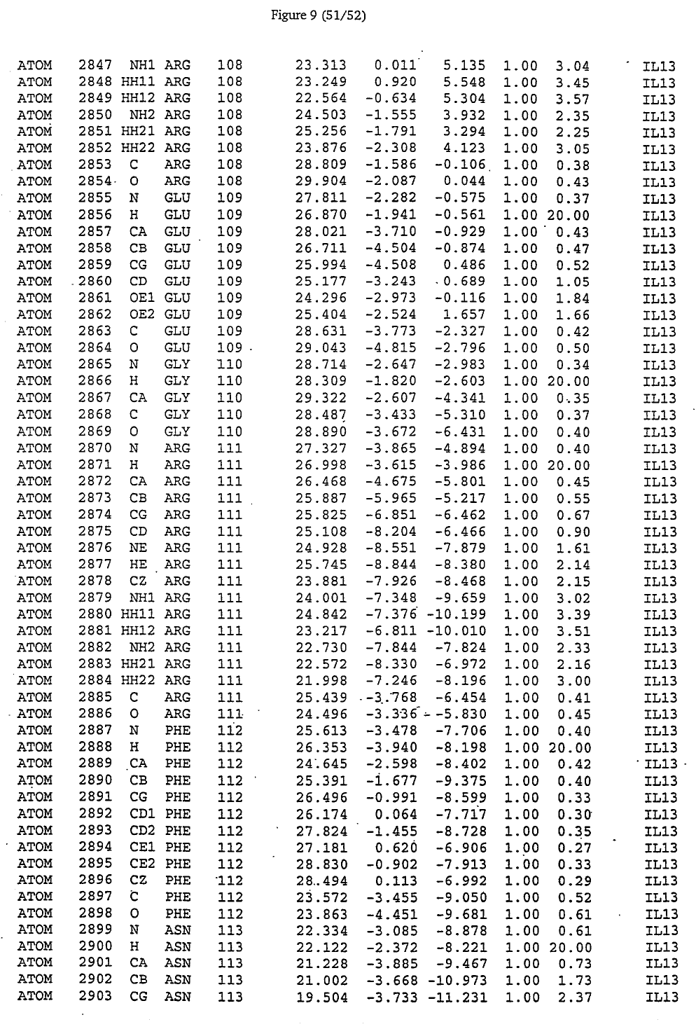

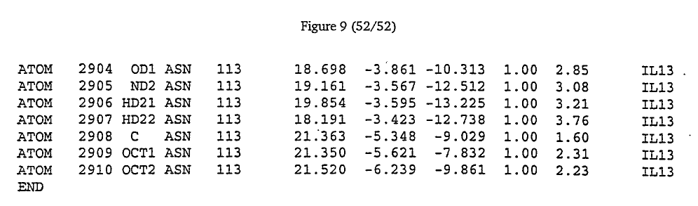

“Structural coordinates” are the Cartesian coordinates corresponding to an atom's spatial relationship to other atoms in a molecule or molecular complex. Structural coordinates may be obtained using x-ray crystallography techniques or NMR techniques, or may be derived using molecular replacement analysis or homology modeling. Various software programs allow for the graphical representation of a set of structural coordinates to obtain a three dimensional representation of a molecule or molecular complex. The structural coordinates of the present invention may be modified from the original set provided in FIG. 8 or 9 by mathematical manipulation, such as by inversion or integer additions or subtractions. As such, it is recognized that the structural coordinates of the present invention are relative, and are in no way specifically limited by the actual x, y, z coordinates of FIG. 8 or 9.

An “agent” shall include a protein, polypeptide, peptide, nucleic acid, including DNA or RNA, molecule, compound or drug.

“Root mean square deviation” is the square root of the arithmetic mean of the squares of the deviations from the mean, and is a way of expressing deviation or variation from the structural coordinates described herein. The present invention includes all embodiments comprising conservative substitutions of the noted amino acid residues resulting in same structural coordinates within the stated root mean square deviation.

“Conservative substitutions” are those amino acid substitutions which are functionally equivalent to the substituted amino acid residue, either by way of having similar polarity, steric arrangement, or by belonging to the same class as the substituted residue (e.g., hydrophobic, acidic or basic), and includes substitutions having an inconsequential effect on the three dimensional structure of IL-13 with respect to the use of said structure for the identification and design of agents that interact with IL-13, for molecular replacement analyses and/or for homology modeling.

An “active site” refers to a region of a molecule or molecular complex that, as a result of its shape and charge potential, favorably interacts or associates with another agent (including, without limitation, a protein, polypeptide, peptide, nucleic acid, including DNA or RNA, molecule, compound, antibiotic or drug) via various covalent and/or non-covalent binding forces. As such, an active site of the present invention may include, for example, the actual site of receptor binding to IL-13, as well as accessory binding sites adjacent to the actual site of receptor binding that nonetheless may affect IL-13 upon interaction or association with a particular agent, either by direct interference with the actual site of receptor binding or by indirectly affecting the steric conformation or charge potential of IL-13 and thereby preventing or reducing receptor binding to IL-13 at the actual site of receptor binding. As used herein, “active site” also includes the receptor site of self association, as well as other binding sites present on IL-13.

A “IL-13 complex” refers to a co-complex of a molecule comprising IL-13 in bound association with a protein, polypeptide, peptide, nucleic acid, including DNA or RNA, small molecule, compound or drug, either by covalent or non-covalent binding forces. A non-limiting example of a IL-13 complex includes the receptor, IL-4Rα bound to IL-13.

The present invention relates to the three dimensional structure of IL-13, and more specifically, to the solution structure of IL-13 as determined using multidimensional NMR spectroscopy and various computer modeling techniques. The structural coordinates of IL-13 in its unbound configuration (FIG. 8) or bound configuration (FIG. 9) are useful for a number of applications, including, but not limited to, the characterization of a three dimensional structure of IL-13, as well as the visualization, identification and characterization of IL-13 active sites, including the site of receptor binding to IL-13. The active site structures may then be used to predict the orientation and binding affinity of a designed or selected agent that interacts with IL-13 or of an IL-13 complex. Accordingly, the invention is also directed to the three dimensional structure of an IL-13 active site, including but not limited to the receptor binding site.

As used herein, the IL-13 in solution comprises amino acid 1-113 of FIG. 2, including conservative substitutions. Preferably, the IL-13 in solution is either unlabeled, ¹⁵N enriched or ¹⁵N, ¹³C enriched, and is preferably biologically active. In addition, the secondary structure of the IL-13 in the solution of the present invention comprises four alpha helices αA, αB, αC and αD, and two beta strands β1 and β2, wherein αA comprises amino acid residues P6-Q22 of IL-13, β1 comprises M33-W35 of IL-13, αB comprises amino acid residues M43-152 of IL-13, αC comprises amino acid residues A59-F70 of IL-13, β2 comprises amino acid residues K89-E91 of IL-13, and αD comprises amino acid residues V92-R108 of IL-13. In the most preferred embodiment, the IL-13 in the solution of the present invention is characterized by a three dimensional structure comprising the complete structural coordinates of the amino acids according to FIG. 8, ± a root mean square deviation from the conserved backbone atoms of said amino acids of not more than 1.5 Å (or more preferably, not more than 1.0 Å, and most preferably, not more than 0.5 Å).

Molecular modeling methods known in the art may be used to identify an active site or binding pocket of IL-13 or of an IL-13 complex. Specifically, the solution structural coordinates provided by the present invention may be used to characterize a three dimensional structure of the IL-13 molecule or molecular complex. From such a structure, putative active sites may be computationally visualized, identified and characterized based on the surface structure of the molecule, surface charge, steric arrangement, the presence of reactive amino acids, regions of hydrophobicity or hydrophilicity, etc. Such putative active sites may be further refined using chemical shift perturbations of spectra generated from various and distinct IL-13 complexes, competitive and non-competitive inhibition experiments, and/or by the generation and characterization of IL-13 or ligand mutants to identify critical residues or characteristics of the active site.

The identification of putative active sites of a molecule or molecular complex is of great importance, as most often the biological activity of a molecule or molecular complex results from the interaction between an agent and one or more active sites of the molecule or molecular complex. Accordingly, the active sites of a molecule or molecular complex are the best targets to use in the design or selection of inhibitors that affect the activity of the molecule or molecular complex.

The present invention is directed to an active site of IL-13 or complex, that, as a result of its shape, reactivity, charge potential, etc., favorably interacts or associates with another agent (including, without limitation, a protein, polypeptide, peptide, nucleic acid, including DNA or RNA, molecule, compound, antibiotic or drug). Preferably, the present invention is directed to an active site of IL-13 that is characterized by the three dimensional structure comprising the relative structural coordinates of amino acid residues A9, E12, E15, E16 and M66 of IL-13 according to FIG. 8 or 9, ± a root mean square deviation from the conserved backbone atoms of said amino acids of not more than 1.5 Å, preferably not more than 1.0 Å, and most preferably not more than 0.5 Å. In another embodiment, the active site of IL-13 is characterized by the three dimensional structure comprising the relative structural coordinates of amino acid residues 152, Q64, R65 and M66 of IL-13 according to FIG. 8 or 9, ± a root mean square deviation from the conserved backbone atoms of said amino acids of not more than 1.5 Å, preferably not more than 1.0 Å, and most preferably not more than 0.5 Å.

In order to use the structural coordinates generated for a solution structure of the present invention as set forth in FIG. 8 or 9, it is often necessary to display the relevant coordinates as, or convert them to, a three dimensional shape or graphical representation, or to otherwise manipulate them. For example, a three dimensional representation of the structural coordinates is often used in rational drug design, molecular replacement analysis, homology modeling, and mutation analysis. This is typically accomplished using any of a wide variety of commercially available software programs capable of generating three dimensional graphical representations of molecules or portions thereof from a set of structural coordinates. Examples of said commercially available software programs include, without limitation, the following: GRID (Oxford University, Oxford, UK); MCSS (Molecular Simulations, San Diego, Calif.); AUTODOCK (Scripps Research Institute, La Jolla, Calif.); DOCK (University of California, San Francisco, Calif.); Flo99 (Thistlesoft, Morris Township, N.J.); Ludi (Molecular Simulations, San Diego, Calif.); QUANTA (Molecular Simulations, San Diego, Calif.); Insight (Molecular Simulations, San Diego, Calif.); SYBYL (TRIPOS, Inc., St. Louis. MO); and LEAPFROG (TRIPOS, Inc., St. Louis, Mo.).

For storage, transfer and use with such programs, a machine, such as a computer, is provided for that produces a three dimensional representation of the IL-13, a portion thereof (such as an active site or a binding site), or a IL-13 complex. The machine of the present invention comprises a machine-readable data storage medium comprising a data storage material encoded with machine-readable data. Machine-readable storage media comprising data storage material include conventional computer hard drives, floppy disks, DAT tape, CD-ROM, and other magnetic, magneto-optical, optical, floptical and other media which may be adapted for use with a computer. The machine of the present invention also comprises a working memory for storing instructions for processing the machine-readable data, as well as a central processing unit (CPU) coupled to the working memory and to the machine-readable data storage medium for the purpose of processing the machine-readable data into the desired three dimensional representation. Finally, the machine of the present invention further comprises a display connected to the CPU so that the three dimensional representation may be visualized by the user. Accordingly, when used with a machine programmed with instructions for using said data, e.g., a computer loaded with one or more programs of the sort identified above, the machine provided for herein is capable of displaying a graphical three-dimensional representation of any of the molecules or molecular complexes, or portions of molecules of molecular complexes, described herein.

In one embodiment of the invention, the machine-readable data comprises the relative structural coordinates of amino acid residues A9, E12, E15, E16 and M66 of IL-13 according to FIG. 8 or 9, ± a root mean square deviation from the conserved backbone atoms of said amino acids of not more than 1.5 Å, or preferably, not more than 1.0 Å, or more preferably not more than 0.5 Å. In an alternate embodiment, the machine-readable data further comprises the relative structural coordinates of amino acid residues 152, Q64, R65 and M66 of IL-13 according to FIG. 8 or 9, ± a root mean square deviation from the conserved backbone atoms of said amino acids of not more than 1.5 Å, preferably not more than 1.0 Å, and most preferably not more than 0.5 Å.

The structural coordinates of the present invention permit the use of various molecular design and analysis techniques in order to (i) solve the three dimensional structures of related molecules, molecular complexes or IIL-13, and (ii) to design, select, and synthesize chemical agents capable of favorably associating or interacting with an active site of an IL-13 molecule, or molecular complex, wherein said chemical agents potentially act as inhibitors, activators, agonists or antagonists of IL-13 or IL-13 binding to a protein, including, but not limited to, a receptor of IL-13 such as IL-4Rα.

More specifically, the present invention provides a method for determining the molecular structure of a molecule or molecular complex whose structure is unknown, comprising the steps of obtaining a solution of the molecule or molecular complex whose structure is unknown, and then generating NMR data from the solution of the molecule or molecular complex. The NMR data from the molecule or molecular complex whose structure is unknown is then compared to the solution structure data obtained from the IL-13 solutions of the present invention. Then, 2D, 3D and 4D isotope filtering, editing and triple resonance NMR techniques are used to conform the three dimensional structure determined from the IL-13 solution of the present invention to the NMR data from the solution molecule or molecular complex. Alternatively, molecular replacement may be used to conform the IL-13 solution structure of the present invention to x-ray diffraction data from crystals of the unknown molecule or molecular complex.

Molecular replacement uses a molecule having a known structure as a starting point to model the structure of an unknown crystalline sample. This technique is based on the principle that two molecules which have similar structures, orientations and positions will diffract x-rays similarly. A corresponding approach to molecular replacement is applicable to modeling an unknown solution structure using NMR technology. The NMR spectra and resulting analysis of the NMR data for two similar structures will be essentially identical for regions of the proteins that are structurally conserved, where the NMR analysis consists of obtaining the NMR resonance assignments and the structural constraint assignments, which may contain hydrogen bond, distance, dihedral angle, coupling constant, chemical shift and dipolar coupling constant constraints. The observed differences in the NMR spectra of the two structures will highlight the differences between the two structures and identify the corresponding differences in the structural constraints. The structure determination process for the unknown structure is then based on modifying the NMR constraints from the known structure to be consistent with the observed spectral differences between the NMR spectra.

Accordingly, in one non-limiting embodiment of the invention, the resonance assignments for the IL-13 solution provide the starting point for resonance assignments of IL-13 in a new IL-13:“unsolved agent” complex. Chemical shift perturbances in two dimensional ¹⁵N/¹H spectra can be observed and compared between the IL-13 solution and the new IL-13:agent complex. In this way, the affected residues may be correlated with the three dimensional structure of IL-13 as provided by the relevant structural coordinates of FIG. 8 or 9. This effectively identifies the region of the IL-13:agent complex that has incurred a structural change relative to the native IL-13 structure. The ¹H, ¹⁵N, ¹³C and ¹³CO NMR resonance assignments corresponding to both the sequential backbone and side-chain amino acid assignments of IL-13 may then be obtained and the three dimensional structure of the new IL-13:agent complex may be generated using standard 2D, 3D and 4D triple resonance NMR techniques and NMR assignment methodology, using the IL-13 solution structure, resonance assignments and structural constraints as a reference. Various computer fitting analyses of the new agent with the three dimensional model of IL-13 may be performed in order to generate an initial three dimensional model of the new agent complexed with IL-13, and the resulting three dimensional model may be refined using standard experimental constraints and energy minimization techniques in order to position and orient the new agent in association with the three dimensional structure of IL-13.

The present invention further provides that the structural coordinates of the present invention may be used with standard homology modeling techniques in order to determine the unknown three-dimensional structure of a molecule or molecular complex. Homology modeling involves constructing a model of an unknown structure using structural coordinates of one or more related protein molecules, molecular complexes or parts thereof (i.e., active sites). Homology modeling may be conducted by fitting common or homologous portions of the protein whose three dimensional structure is to be solved to the three dimensional structure of homologous structural elements in the known molecule, specifically using the relevant (i.e., homologous) structural coordinates provided by FIG. 8 or 9 herein. Homology may be determined using amino acid sequence identity, homologous secondary structure elements, and/or homologous tertiary folds. Homology modeling can include rebuilding part or all of a three dimensional structure with replacement of amino acids (or other components) by those of the related structure to be solved.

Accordingly, a three dimensional structure for the unknown molecule or molecular complex may be generated using the three dimensional structure of the IL-13 molecule of the present invention, refined using a number of techniques well known in the art, and then used in the same fashion as the structural coordinates of the present invention, for instance, in applications involving molecular replacement analysis, homology modeling, and rational drug design.

Determination of the three dimensional structure of IL-13, its binding site to a receptor, and other binding sites, is critical to the rational identification and/or design of agents that may act as inhibitors, activators, agonists or antagonists of IL-13. This is advantageous over conventional drug assay techniques, in which the only way to identify such an agent is to screen thousands of test compounds until an agent having the desired inhibitory effect on a target compound is identified. Necessarily, such conventional screening methods are expensive, time consuming, and do not elucidate the method of action of the identified agent on the target compound.

However, advancing X-ray, spectroscopic and computer modeling technologies allow researchers to visualize the three dimensional structure of a targeted compound (i.e., of IL-13). Using such a three dimensional structure, researchers identify putative binding sites and then identify or design agents to interact with these binding sites. These agents are then screened for an inhibitory effect upon the target molecule. In this manner, not only are the number of agents to be screened for the desired activity greatly reduced, but the mechanism of action on the target compound is better understood.

Accordingly, the present invention further provides a method for identifying an agent that interacts with IL-13, comprising the steps of generating the three dimensional structure of IL-13 as defined by the relative structural coordinates of FIG. 8 or 9, and using that three dimensional structure to identify, design or select an agent that interacts with IL-13. The inhibitor may be selected by screening an appropriate database, may be designed de novo by analyzing the steric configurations and charge potentials of an empty IL-13 or IL-13 complex active site in conjunction with the appropriate software programs, or may be designed using characteristics of known agents in order to create “hybrid” agents.

An agent that interacts or associates with an active site of IL-13 or an IL-13 complex may be identified by determining an active site from the three dimensional structure of IL-13, and performing computer fitting analyses to identify an agent which interacts or associates with said active site. Computer fitting analyses utilize various computer software programs that evaluate the “fit” between the putative active site and the identified agent, by (a) generating a three dimensional model of the putative active site of a molecule or molecular complex using homology modeling or the atomic structural coordinates of the active site, and (b) determining the degree of association between the putative active site and the identified agent. The degree of association may be determined computationally by any number of commercially available software programs, or may be determined experimentally using standard binding assays.

Three dimensional models of the putative active site may be generated using any one of a number of methods known in the art, and include, but are not limited to, homology modeling as well as computer analysis of raw structural coordinate data generated using crystallographic or spectroscopy techniques. Computer programs used to generate such three dimensional models and/or perform the necessary fitting analyses include, but are not limited to: GRID (Oxford University, Oxford, UK), MCSS (Molecular Simulations, San Diego, Calif.), AUTODOCK (Scripps Research Institute, La Jolla, Calif.), DOCK (University of California, San Francisco, Calif.), Flo99 (Thistlesoft, Morris Township, N.J.), Ludi (Molecular Simulations, San Diego, Calif.), QUANTA (Molecular Simulations, San Diego, Calif.), Insight (Molecular Simulations, San Diego, Calif.), SYBYL (TRIPOS, Inc., St. Louis. MO) and LEAPFROG (TRIPOS, Inc., St. Louis, Mo.).

In the preferred embodiment, the method of the present invention includes the use of an active site characterized by the three dimensional structure comprising the relative structural coordinates of amino acid residues A9, E12, E15, E16 and M66 of IL-13 according to FIG. 8 or 9, ± a root mean square deviation from the conserved backbone atoms of said amino acids of not more than 1.5 Å, preferably not more than 1.0 Å, and most preferably not more than 0.5 Å. In another embodiment, the active site is characterized by the three dimensional structure comprising the relative structural coordinates of amino acid residues 152, Q64, R65 and M66 of IL-13 according to FIG. 8 or 9, ± a root mean square deviation from the conserved backbone atoms of said amino acids of not more than 1.5 Å, preferably not more than 1.0 Å, and most preferably not more than 0.5 Å. It is understood that the method of the present invention includes additional embodiments comprising conservative substitutions of the noted amino acids which result in the same structural coordinates of the corresponding residues in FIG. 8 or 9 within the stated root mean square deviation.

The effect of such an agent identified by computer fitting analyses on IL-13 or the IL-13 complex may be further evaluated computationally, or experimentally by competitive binding experiments or by contacting the identified agent with IL-13 (or a IL-13 complex) and measuring the effect of the agent on the target's biological activity. Standard assays may be performed and the results analyzed to determine whether the agent is an activator, inhibitor, agonist or antagonist of IL-13 activity (e.g., the agent may reduce or prevent binding affinity between IL-13 and a relevant binding protein).

An agent designed or selected to interact with IL-13 preferably is capable of both physically and structurally associating with IL-13 via various covalent and/or non-covalent molecular interactions, and of assuming a three dimensional configuration and orientation that complements the relevant active site of IL-13.

Accordingly, using these criteria, the structural coordinates of the IL-13 molecule as disclosed herein, and/or structural coordinates derived therefrom using molecular replacement or homology modeling, agents may be designed to increase either or both of the potency and selectivity of known inhibitors, either by modifying the structure of known inhibitors or by designing new agents de novo via computational inspection of the three dimensional configuration and electrostatic potential of a IL-13 active site.

Accordingly, in one embodiment of the invention, the structural coordinates of FIG. 8 or 9 of the present invention, or structural coordinates derived therefrom using molecular replacement or homology modeling techniques as discussed above, are used to screen a database for agents that may act as potential activators, inhibitors, agonists or antagonists of IL-13 activity. Specifically, the obtained structural coordinates of the present invention are read into a software package and the three dimensional structure is analyzed graphically. A number of computational software packages may be used for the analysis of structural coordinates, including, but not limited to, Sybyl (Tripos Associates), QUANTA and XPLOR (Brunger, A. T., (1994) X-Plor 3.851: a system for X-ray Crystallography and NMR. Xplor Version 3.851 New Haven, Conn.: Yale University Press). Additional software programs check for the correctness of the coordinates with regard to features such as bond and atom types. If necessary, the three dimensional structure is modified and then energy minimized using the appropriate software until all of the structural parameters are at their equilibrium/optimal values. The energy minimized structure is superimposed against the original structure to make sure there are no significant deviations between the original and the energy minimized coordinates.

The energy minimized coordinates of IL-13 bound to a “solved” agent are then analyzed and the interactions between the solved ligand and IL-13 are identified. The final IL-13 structure is modified by graphically removing the solved agent so that only IL-13 and a few residues of the solved agent are left for analysis of the binding site cavity. QSAR and SAR analysis and/or conformational analysis may be carried out to determine how other inhibitors compare to the solved inhibitor. The solved agent may be docked into the uncomplexed structure's binding site to be used as a template for data base searching, using software to create excluded volume and distance restrained queries for the searches. Structures qualifying as hits are then screened for activity using standard assays and other methods known in the art.

Further, once the specific interaction is determined between the solved agent, docking studies with different agents allow for the generation of initial models of new agents bound to IL-13. The integrity of these new models may be evaluated a number of ways, including constrained conformational analysis using molecular dynamics methods (i.e., where both IL-13 and the bound agent are allowed to sample different three dimensional conformational states until the most favorable state is reached or found to exist between the protein and the bound agent). The final structure as proposed by the molecular dynamics analysis is analyzed visually to make sure that the model is in accord with known experimental SAR based on measured binding affinities. Once models are obtained of the original solved agent bound to IL-13 and computer models of other molecules bound to IL-13, strategies are determined for designing modifications into the inhibitors to improve their activity and/or enhance their selectivity.

Once an IL-13 binding agent has been optimally selected or designed, as described above, substitutions may then be made in some of its atoms or side groups in order to improve or modify its selectivity and binding properties. Generally, initial substitutions are conservative, i.e., the replacement group will have approximately the same size, shape, hydrophobicity and charge as the original group. Such substituted chemical compounds may then be analyzed for efficiency of fit IL-13 by the same computer methods described in detail above.

With respect to agonist/antagonist design, there are a number of computational software packages that may be used for the analysis of protein NMR structures. In this case, the software packages Sybyl v.6.4+ to v.6.5+ from Tripos Associates and QUANTA97 (Version 97.1003) an CPLOR (Version 3.840) from MSI may be used. Once the coordinates have been determined by NMR a number of steps may be taken as listed below:

1. The original coordinates are read into the software package and the structure(3D) is analyzed graphically. In addition, programs within QUANTA check for the correctness of the NMR coordinates with regard to features such as bond and atom types.

2. The modified (if necessary) structure is energy minimized using the QUANTA/CHARMm until all the structural parameters are at their equilibrium/optimal values.

3. The energy minimized structure is superimposed against the original NMR structure to ensure there are no significant deviations between the original and minimized coordinates.

4. The protein-native complex is analyzed, the interactions between the native ligand and the protein are identified. The uncomplexed structure binding site is compared to the complexed structure's binding site for areas which may be exploited by a potential agonist/antagonist.

5. The final protein structure bound to the native ligand is modified by removing the native ligand so only the protein and a few residues of the natural ligand are left for analysis of the binding site cavity. The natural ligand residues are docked into the uncomplexed structure's binding site to be used as templates for SYBYL/UNITY database searching.

6. SYBYL/UNITY is used to create excluded volume and distance constrained queries for searching structural databases. Structural qualifying as ‘hits’ are screened for activity.

7. Once specific ligand-protein interactions are determined between new ligands and the protein structure, docking studies may be carried out between the different series of in-house ligands and IL-13. This part gives us the initial modeled complexes of new ligands with IL-13.

To check for the integrity of the modeled new IL-13-ligand complexes, different procedures may be used. In this case, constrained conformational analysis is carried out using molecular dynamic methods. In this modeling process, both protein and the complexed ligand are allowed to sample different 3D conformational states until the most favorable state is reached or found to exist between protein and inhibitor. The final structure as proposed by the molecular dynamics analysis is analyzed visually to make sure the modeled complex is in accord with known experimental SAR based on measured binding affinities.

Furthermore, agonist/antagonist design may take advantage of either the IL-13 binding chain or IL-4Rα binding states. Additionally, details of the IL-13/IL-4Rα interface may be used to design ligands that effectively bind to either IL-13 or IL-4Rα in this binding region. Also, conformational changes in IL-4 upon binding the IL-13 binding chain that are required to recruit IL-4Rα may be utilized in designing ligands that either inhibit or promote this structural change to affect the inherent IL-13 activity. Once computer models of the native ligand and/or other ligands bound to IL-13 have been determined, modifications can be designed into ligands to improve binding and/or activity based upon the models.

Various molecular analysis and rational drug design techniques are further disclosed in U.S. Pat. Nos. 5,834,228, 5,939,528 and 5,865,116, as well as in PCT Application No. PCT/US98/16879, published as WO 99/09148, the contents of which are hereby incorporated by reference.

The present invention may be better understood by reference to the following non-limiting Examples. The following Examples are presented in order to more fully illustrate the preferred embodiments of the invention, and should in no way be construed as limiting the scope of the present invention.

### EXAMPLE 1

A. Methods and Methods.

The uniform ¹⁵N and ¹³C labeled 113 amino acid IL-13 was obtained as follows. The cDNA encoding the mature secreted portion of IL-13 was reconstructed with silent changes that optimized E. coli codon usage and increased AT content at the 5-prime end. The gene was subcloned into the T7-lac vector pRSET for expression in Escherichia coli BL21(DE3). Growth and expression were at 37°, in shake flasks with minimal medium supplemented with ¹³C-glucose and/or ¹⁵N-ammonium sulfate. The protein was essentially completely insoluble. Cells were broken with a microfluidizer and insoluble IL-13 was collected and dissolved at about 2 mg/ml in 50 mM CHES (pH 9), 6 M guanidine-HCl, 1 mM EDTA, 20 mM DTT. The solution was diluted 20-fold into 50 mM CHES (pH 9), 3 M guanidine-HCl 100 mM NaCl, 1 mM oxidized glutathione and dialyzed twice against 10 volumes of 50 mM CHES (pH 9), 100 mM NaCl and once against 10 volumes of 20 mM MES (pH 6). Following clarification by centrifugation, IL-13 was adsorbed to SP-Sepharose and eluted with a gradient of NaCl in MES buffer. Final purification was by size-exclusion chromatography in 40 mM sodium phosphate, 40 mM NaCl on Superdex 75.

The NMR samples contained 1 mM of IL-13 in a buffer containing 40 mM sodium Phosphate, 2 mM NaN₃, 40 m M NaCl, in either 90% H2O/10% D20 or 100% D20 at pH 6.0. All NMR spectra were recorded at 25° C. on a Bruker DRX 600 spectrometer equipped with a triple-resonance gradient probe. Spectra were processed using the NMRPipe software package (Delaglio et al., 1995) and analyzed with PIPP(Garrett et al., 1991).

The assignments of the ¹H, ¹⁵N, ¹³Co, and ¹³C resonances were based on the following experiments: CBCA(CO)NH, CBCANH, C(CO)NH, HC(CO)NH, HNHB, HNCO, HNHA, HNCA and HCCH-COSY (for reviews see (Bax et al., 1994; Clore and Gronenbom, 1994). The accuracy of the IL-13 NMR assignments was further confirmed during the structure calculation and by sequential NOEs in the ¹⁵N-edited NOESY-HMQC spectra and by NOEs between the β-strands observed in the ¹³C-edited NOESY-HMQC and ¹⁵N-edited NOESY-HMQC spectra.

The present structure is based on the experimental distance and torsional angle restraints determined from the following series of spectra: HNHA (Vuister and Bax, 1993), HNHB (Archer et al., 1991), HACAHB-COSY (Grzesiek et al., 1995), 3D ¹⁵N- (Marion et al., 1989; Zuiderweg and Fesik, 1989) and ¹³C-edited NOESY (Ikura et al., 1990; Zuiderweg et al., 1990). The ¹⁵N-edited NOESY, and ¹³C-edited NOESY experiments were collected with 100 ms and 120 ms mixing times, respectively.

The β-methylene stereospecific assignments and χ1 torsion angle restraints were obtained primarily from a qualitative estimate of the magnitude of ³J_{αβ} coupling constants from the HACAHB-COSY experiment (Grzesiek et al., 1995) and ³J_{Nβ} coupling constants from the HNHB experiment (Archer et al., 1991). Val γ-methyl stereospecific assignments were made from the relative inte nsity of intraresidue NH-CγH and CαH-CγH NOEs (Zuiderweg et al., 1985). Leu and Ile χ2 torsion angle restraints and Leu δ-methyl stereospecific assignments were obtained primarily from ¹³C-¹³C-long range coupling constants (Bax and Pochapsky, 1992) and the relative intensity of intra-molecular NOEs (Powers et al., 1993). The φ and ψ torsion angle restraints were obtained from ³J_{NHα} coupling constants measured from the HNHA experiment (Vuister and Bax, 1993) and from chemical shift analysis using the TALOS program (Cornilescu et al., 1999). The minimum ranges employed for the φ, ψ, and χ torsion angle restraints were ±30°, ±50°, and ±20°, respectively. The NOEs assigned from the 3D ¹⁵N- and ¹³C-edited NOESY experiments were classified into strong, medium, weak and very weak corresponding to interproton distance restraints where non-stereospecifically assignments were corrected appropriately for center averaging (Wuthrich et al., 1983).

The structures were calculated using the hybrid distance geometry-dynamical simulated annealing method of Nilges et al., (1988c) with minor modifications (Clore et al., 1990) using the program XPLOR (Brunger, 1993), adapted to incorporate pseudopotentials for ³J_{NHα} coupling constants (Garrett et al., 1994), secondary ¹³C^{α}/¹³C^{β} chemical shift restraints (Kuszewski et al., 1995) radius of gyration (Kuszewski et al., 1999), and a conformational database potential (Kuszewski et al., 1996; Kuszewski et al., 1997). The target function that is minimized during restrained minimization and simulated annealing comprises only quadratic harmonic terms for covalent geometry, ³^{_{J}}NHα coupling constants and secondary ¹³C^{α}/¹³C^{β} chemical shift restraints, square-well quadratic potentials for the experimental distance, radius of gyration and torsion angle restraints, and a quartic van der Waals term for non-bonded contacts. All peptide bonds were constrained to be planar and trans. There were no hydrogen-bonding, electrostatic, or 6-12 Lennard-Jones empirical potential energy terms in the target function. The radius of gyration can be predicted with reasonable accuracy on the basis of the number of residues using a relationship determined empirically from the analysis of high-resolution x-ray structures (Kuszewski et al., 1996). The force constant for the conformational database and radius of gyration potentials were kept relatively low throughout the simulation to allow the experimental distance and torsional angle restraints to be the predominant influences on the resulting structures. The force constant for the NOE and dihedral restraints was 30 times and ten times stronger then the force constants used for the conformational database and radium gyration potentials, respectively.

Overlay of the IL-3 solution structure with free IL-4 and IL-4 in the IL-4/IL4Rα complex was accomplished with Quanta (Molecular Simulations, Inc., San Diego, Calif.). Minimization of the IL-3 side-chains to remove steric clashes was accomplished with CHARMM (Molecular Simulations, Inc., San Diego, Calif.). Measurement of the interhelical angles and axial distances in the IL-13 and IL-4 structures was determined using INTERHLX.

Atomic coordinates for the 30 final simulated annealing structures and the restrained minimized mean structure and the NMR chemical shift assignments of IL-13 have been deposited in the RCSB Protein Data Bank (PDB ID: 1ijz and 1iko) and the BioMagResBank (BMRB-5004), respectively.

B. Results and Discussion

1. IL-13 NMR Structure

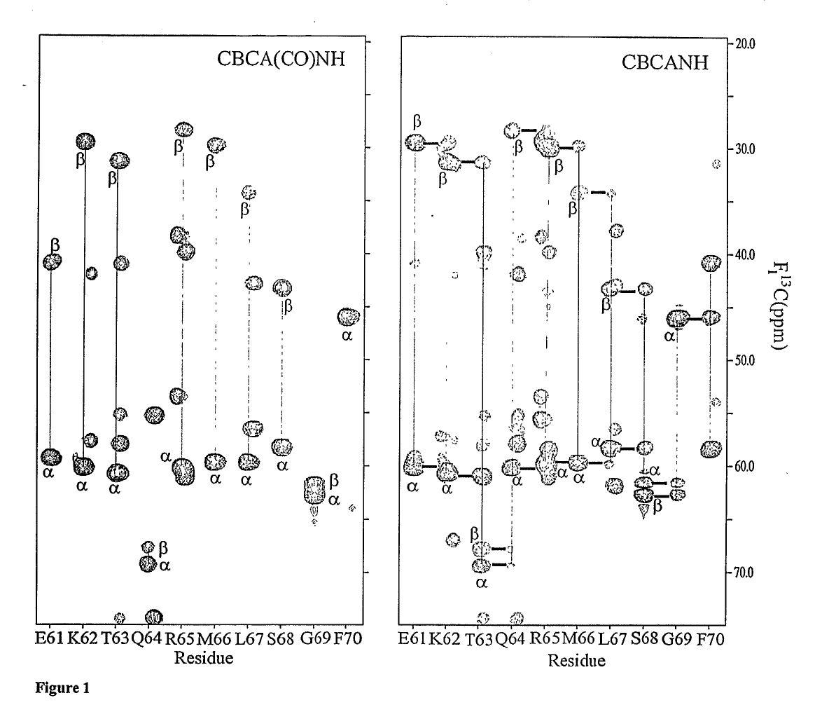

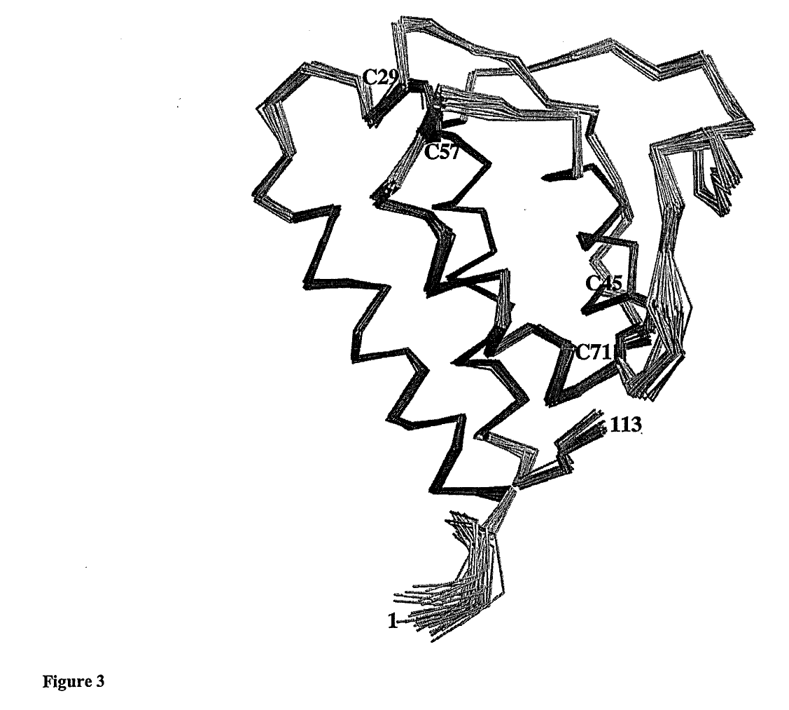

Nearly complete backbone and side-chain ¹H, ¹⁵N, ¹³C, and ¹³CO assignments have been obtained for IL-13 that enabled the determination of a high-resolution solution structure for the protein by NMR (FIG. 1). The IL-13 structure is well defined by the NMR data, where a total of 2848 constraints were used to refine the structure (FIG. 2). This is evident by a best fit superposition of the backbone atoms shown in FIG. 3, where the atomic r.m.s. distribution of the 30 simulated annealing structures about the mean coordinate positions for residues 1-113 is 0.43 (±0.04) Å for the backbone atoms (Table 1). All of the backbone torsion angles for non-glycine residues lie within expected regions of the Ramachandran plot where 89.9% of the residues lie within the most favored region of the Ramachandran φ, ψ plot, 9.1% in the additionally allowed region and 1.0% in the generously allowed region. The high quality of the IL-13 NMR structure is also evident by the results of the PROCHECK analysis, where an overall G-factor of 0.15, a hydrogen bond energy of 0.90 and only 1.8 bad contacts per 100 residues were determined. The calculated PROCHECK parameters for IL-13 are comparable to values obtainable with ˜1 Å X-ray structures and implies a relatively high quality for the structure, but does not infer an inherent resolution (Laskowski et al., 1996).

The IL-13 protein adopts the expected left-handed four-helical bundle with up-up-down-down connectivities previously observed for IL-4 and similar cytokines. The four helical regions correspond to residues 6-22 (α_{A}); 43-52 (α_{B}); 59-70 (α_{C}) and 92-108 (α_{D}). The observed angles and axial separation between the four antiparallel helical pairs, α_{A}-α_{C}, α_{C}-α_{B}, α_{B}-α_{D} and α_{D}-α_{A}, are −161.7° and 11.3 Å, −147.7° and 9.2 Å, −165.1° and 12.7 Å, and −150.3° and 9.8 Å, respectively. The corresponding values between the two parallel helical pairs, α_{A}-α_{B} and α_{C}-α_{D}, are 37.0° and 16.4 Å, and 33.4° and 14.2 Å, respectively. In addition, a short β-sheet region was observed in the IL-13 structure which corresponds to residues 33-35 (β₁) and 89-91 (β₂). Additionally, distinct Cβ chemical shifts (˜42 ppm) for three Cys residues confirmed the presence of two disulfide bonds in the IL-13 structure. The C^{β} chemical shift for C29 was anomalous, where the chemical shift (34 ppm) was in-between typical values for both oxidized and reduced forms. The further identification of the C29-C57 and C45-C71 disulfide bonds was determined by distinct intermolecular NOEs that were identified during the IL-13 structure calculation. In particular, C29 H^{α} to C57 H^{β}, C29 H^{β} to C57 H^{β} NOEs and C45 H^{α} to C71 H^{β}, C45 H^{β} to C71 H^{β} NOEs defined the C29-C57 and C45-C71 disulfide bonds, respectively.

An interesting observation for the IL-13 structure is the presence of chemical shift heterogeneity in the 2D ¹H-¹⁵N HSQC spectra for residues in the structural vicinity of C29. In addition to residues sequential to C29 and C57, A93 and residues sequential to A93 also exhibited multiple ¹H-¹⁵N HSQC peaks. Except for the backbone NH resonance assignments, the remainder of the spin-system chemical shifts assignments for these residues were essentially identical. More importantly, the 3D ¹⁵N-edited NOESY spectra exhibited identical NOE patterns and relative intensities for the multiple backbone NH diagonal peaks. Therefore, the chemical shift multiplicity observed in the 2D ¹H-¹⁵N HSQC spectra suggests a local conformational heterogeneity in the vicinity of C29, where the IL-13 structural change is within the resolution of the structure and the limits of detection for an NOE intensity change. A probable source for the structural heterogeneity is the presence of multiple conformations for the side-chain dihedral angles that comprise the C29-C57 disulfide bond. The C^{α}-C^{α} distance separation for the two cysteins involved in a disulfide bond are directly dependent on the side-chain dihedral angles (Richardson, 1981). A distance range of 4.4 to 6.8 Å for the C^{α}-C^{α} separation is observed for typical values of dihedral angles observed in a disulfide bond, but distance changes of only 0.1-0.2 Å is common between pairs of side-chain conformations. Most likely, a smaller distance change is the source of the heterogeneity present in IL-13 where the different side-chain conformations result in C29 being slightly closer to either C57 or A93. The conformational heterogeneity centered on C29 may also explain the anomalous Cβ chemical shift for this residue.

Another feature of the IL-13 structure is the presence of three long loops connecting the four helices. The shortest loop connects helices α_{B}, and α_{C} and comprises residues N53 to S58. The two long overhand connections are comprised of residues N23 to G42, which connects helices α_{A} with α_{B}, and residues C71 to E91, which connects α_{C} with α_{D}. These loops come in close contact to form the short β-sheet. Additionally, the C29-C57 disulfide bond connects the AB loop to the BC loop. The combination of the short β-sheet and the disulfide bond results in regions of these loops being relatively well defined. Further stability of the long loops occurs from long-range intermolecular NOEs that result in packing of parts of the loop against the helical bundle.

An another interesting feature of the IL-13 structure is the location of the C45-C71 disulfide bond, which effectively connects the N-terminus of α_{B} with the C-terminus of α_{C}. Since the α_{B} and α_{C} helices are also connected by the short BC loop, which is further stabilized by the C29-C57 disulfide bond, the orientation of the α_{B} and α_{C} helices is extensively defined by covalent connectivity. The end result is a closed loop connecting the α_{B} and α_{C} helices.

2. Comparison of the IL-13 and IL-4 Solution Structures

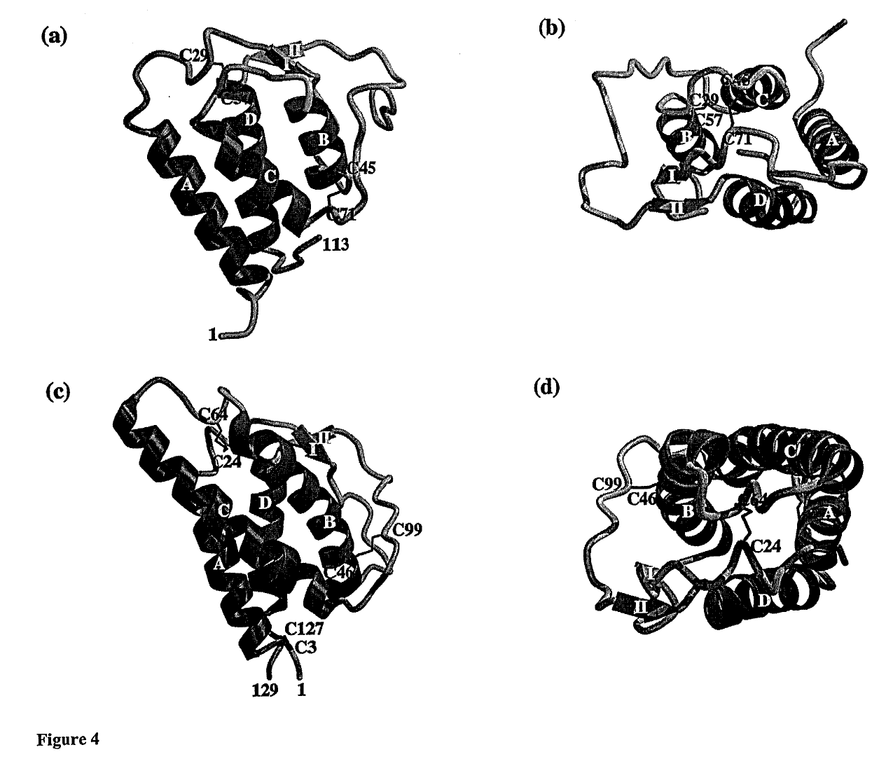

An abundance of structural information for IL-4 has been previously determined by both NMR and X-ray crystallography where a reasonable consensus was obtained for the IL-4 structure (Smith et al., 1994). Therefore, a single solution structure of IL-4 (PDB ID: 1BBN) was used to simplify the comparison between the solution structure of IL-13 with IL-4 (Powers et al., 1992; Powers et al., 1993). Ribbon diagrams for both the IL-13 and IL-4 restrained minimized solution structures are shown in FIG. 4. While the overall folding topology of the two proteins is quite similar, there are obvious distinctions between the two structures. A primary distinction is the overall size difference between the two proteins. IL-4 contains a total of 129 residues compared to 113 for IL-13. This results in the extension of the IL-4 structure by ˜12 Å along the long axis relative to IL-13. Consistent with the overall size difference, are variations in the helix lengths between the IL-4 and IL-13 structures. The length of the four helices in IL-4 corresponds to 17, 23, 26 and 16 residues for helices α_{A}, α_{B}, α_{C} and α_{D}, respectively. Conversely, in IL-13 helices α_{A}, α_{B}, α_{C} and α_{D} have lengths of 17, 10, 12 and 17 residues, respectively. Clearly, the most pronounced distinction is between helices α_{B} and α_{C}, where the IL-4 helices are more than double the length of IL-13. Interestingly, a similar difference in the loop regions between the helices was not seen. The length of the AB, BC and CD loops between IL-4 and IL-13 are identical or nearly identical, where the loops in IL-13 are longer by one to two residues. Similarly, the length of the short β-sheet that comprises part of loops AB and CD are essentially identical. Another distinction between the two protein structures is the number and location of the disulfide bonds. IL-4 has a total of three disulfide bonds that connect the N- and C-terminus (C3-C127), the AB and BC loops (C24-C64), and helix α_{B} to loop CD (C46-C99). Conversely, IL-13 contains only two disulfide bonds that connect the AB and BC loops (C29-C57) and helix α_{B} to helix α_{C} (C45-C71).

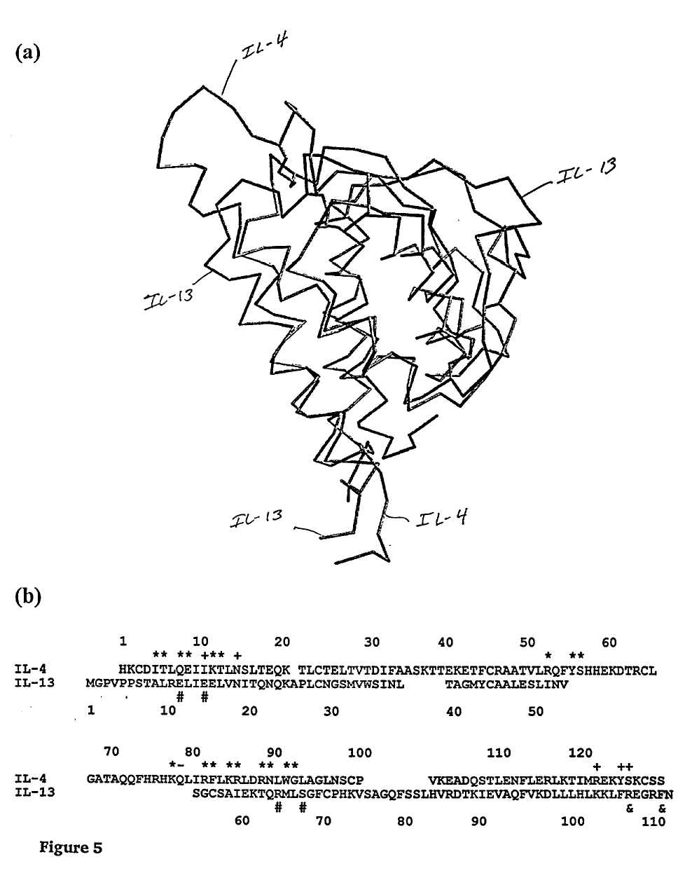

A 25% sequence homology exists between IL-13 and IL-4; however, optimal superposition of the two proteins is determined mainly by alignment of shared secondary structure elements. An overlay of the IL-13 and IL-4 solution structures based on the common secondary structure elements and Cys residues yielded a backbone r.m.s. of 1.44 Å. The sequential alignment based on the shared secondary structure elements and Cys residues along with the overlay of the backbone atoms for IL-13 with IL-4 is illustrated in FIG. 5. In general, there is a good agreement in the superposition between the IL-13 and IL-4 structures including the loop regions. Nevertheless, there exist some distinct differences between the two proteins in the relative orientations and packing of the four-helix bundle, where aB and aD exhibit the largest changes. This is exemplified by an observed 20° difference in the interhelical angle between helices α_{B}-α_{D} and changes in opposite directions in the axial separation for helices α_{A}-α_{B} and α_{C}-α_{D}. The α_{A}-α_{B} axial separation decreases from 16.4 Å to 12.6 Å between IL-13 and IL-4, respectively. Conversely, the α_{C}-α_{D} axial separation increases from 14.2 Å to 16.7 Å between IL-13 and IL-4. Since α_{B} in IL-13 is the shortest helix and half the length of α_{B} in IL-4, the observed structural changes may be attributed to this change in helix length. Furthermore, the relative orientation of α_{B} in IL-13 is also defined by the disulfide bonds at both the N- and C-terminal ends of the helix. Comparison of IL-13 with IL-4 indicates that only a partial spatial alignment of the conserved cysteins occurs, further contributing to the perturbation in α_{B}. There is a good agreement with the relative orientation of C57 from IL-13 with C46 from IL-4. To a lesser extent, the positioning of C29 from IL-13 agrees with C24 from IL-4. But, there is essentially no correlation between the other members of the disulfide pairs. This difference results from the shorter α_{B} and α_{C} helices in IL-13 and that C99 resides within the CD loop in IL-4 compared to C71 being located in helix C for IL-13. Despite the highlighted differences between IL-13 and IL-4, it is important to stress that the overlap of the protein folds for the two proteins is quite similar.

3. Implication for IL-13 Receptor Binding

The recent X-ray crystal structure of IL-4 complexed to the IL-4 receptor α chain has provided insight into cytokine-receptor interactions. Furthermore, an abundance of prior mutational work provides additional information pertaining to the characteristics of the cytokine-receptor interactions. A strong overlap in functionality exists for both IL-13 and IL-4 that is further exemplified by the fact that both receptors contain the same IL-4 chain. Therefore, the combination of the observed similarity in the protein folds, mutational data and the IL-4/receptor complex provides a framework to investigate the interaction of IL-13 with its receptor.

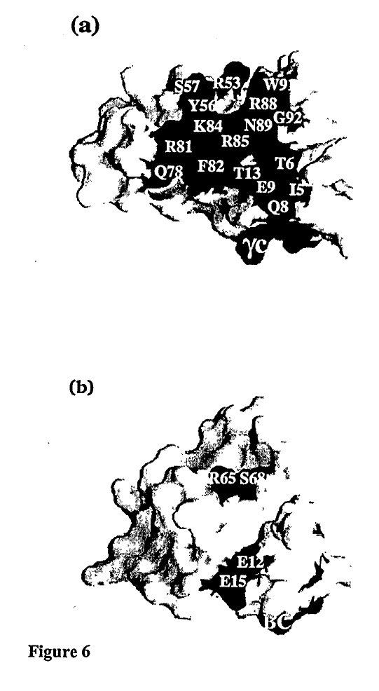

A combination of mutational and kinetic analysis has identified a distinct site on the IL-4 structure associated with IL-4Rα binding and a second site associated with signaling through the γc chain (Wang et al., 1997, Kruse et al., 1993, Letzelter et al., 1998). The IL-4Rα binding site on IL-4 is associated with amino acids that comprise a surface formed by helices A (15, E9, T13) and C (K77, R81, K84, R85 R88, N89, W91). The second site associated with signaling through the γc chain corresponds to residues in helices A (I11, N15) and D (R121, Y124, S125). Similar mutational work on IL-13 that alters its reactivity to IL-13R has also identified amino acids in helices A (E12, E15), C (R65, S68) and D (R108, F112), based on the predicted secondary structure for IL-13 (Thompson et al., 1999, Oshima et al., 2000). The results of the mutational analysis were mapped onto a GRASP surface for both IL-4 and IL-13 (FIGS. 6a and 6c). This analysis identifies the potential IL-13 binding chain and IL-4Rα binding sites on IL-13, which are consistent with the IL-4 binding sites.

The X-ray structure of IL-4 complexed to IL-4Rα confirmed the previous mutational data in identifying the α-chain binding site on IL-4 while further elucidating the specifics of the protein-receptor interaction (Hage et al., 1999). The face of helices α_{A} and α_{C} from IL-4 are almost perpendicular to the L-shaped structure of IL-4Rα. Contact residues from IL-4 are predominately polar and charged residues while the complementary receptor epitope is composed of clusters of polar residues surrounded by hydrophobic residues. Three distinct clusters of residues are described where E9 and R88 from IL-4 are focal points in clusters I and II, respectively, where these residues are involved in hydrogen bonds and ionic bonds with numerous IL-4Rα residues. A number of additional IL-4 residues proximal to E9 and R88 complete the IL-4-receptor interface. The third cluster is described as primarily an electrostatic interaction that does not significantly contribute to the binding affinity, but facilitates complex formation. An overlay of the backbone atoms of IL-13 with IL-4 based primarily on a correlation of secondary structure elements provided a mechanism to establish a structure-based sequence alignment. This structure-based sequence alignment of IL-13 with IL-4 is shown in FIG. 4, where both the mutational data and the key contact residues from the IL-4/receptor X-ray structure is summarized. Again, there is a clear consistency between the IL-4 mutational and structure contact data, where the IL-13 mutational data correlates well with this information. The overlay of the IL-13 structure with IL-4 may then be used in a similar manner to create a model of IL-13 complexed with IL-4Rα.

By creating a best-fit superposition of IL-13 with IL-4 in the IL-4/IL-4Rα complex, a simple model of IL-13 complexed with IL-4Rα is obtained. An overlay of the IL-13 NMR structure with IL-4 from the IL-4/receptor X-ray structure based on the common secondary structure elements and Cys residues yielded a backbone r.m.s. of 1.55 Å. Additional refinement of the IL-13/IL-4Rα complex was limited to minimization of the IL-13 side-chain conformations to remove obvious steric clashes between IL-13 and IL-4Rα.

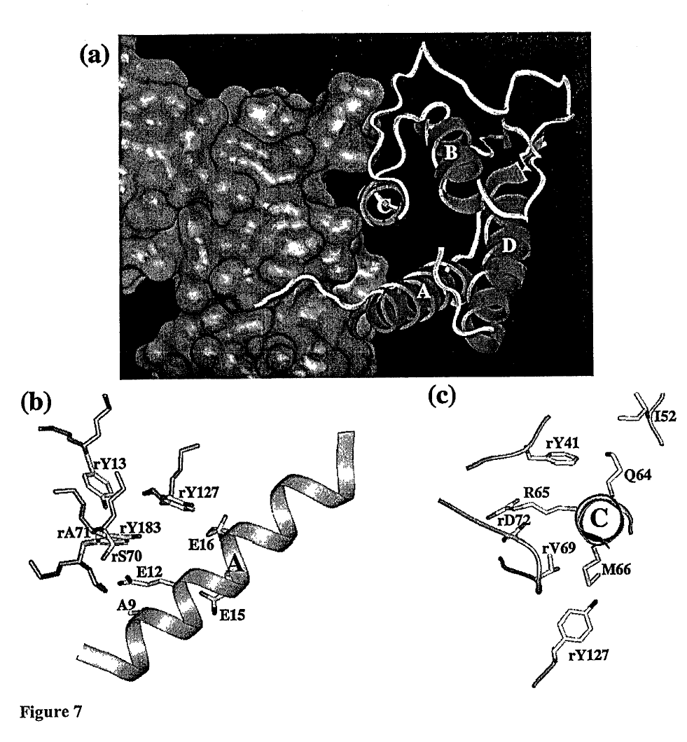

The IL-13/IL-4Rα model is illustrated in FIG. 7. It is readily apparent that the general interaction of IL-13 closely mimics the IL-4/IL-4Rα complex. Particularly, helices α_{A} and α_{C} pack approximately perpendicular against IL-4Rα (FIG. 7A). Furthermore, the framework of the IL-13 side-chain interactions with IL-4Rα mimics the network of interactions observed in the IL-4/IL-4Rα complex. In particular, E12 from IL-13 is positioned to mimic the bonding network of E9 from IL-4 with Y13, Y183 and S70 from IL-4Rα (FIG. 7B). Similarly, R65 from IL-13 is reasonably positioned to form a potential salt bridge with D72 from IL-4R (FIG. 7C). This interaction is comparable to the interaction of R88 from IL-4 with D72 from IL-4Rα. Distinctions between the IL-13/IL-4Rα model relative to the IL-4/IL-4Rα X-ray structure becomes apparent when comparison of the binding network that complement the E12 and R65 interaction with IL-4Rα is made. By reference to IL-4, residues proximal to E12 that are predicted to interact with IL-4Rα consist of IL-13 residues A9, E15, E16 and M66. These residues would correlate with T6, K12, T13 and N89 from IL-4 and interact with S70, Y183, Y127 and A71, respectively (FIG. 7B). Correspondingly, residues near R65 that are predicted to bind IL-4Rα comprises IL-13 residues 152, Q64 and M66 which correlate with IL-4 residues R53, N89 and W91. These IL-4 residues were shown to interact with F41 and V69 from IL-4R (FIG. 7C). While some comparable interactions are potentially present in the IL-13/IL-4α models, these interactions are clearly not optimal. Also, there exist some polarity or charge changes that would be predicted to have a detrimental affect on the affinity of IL-13 with IL-4Rα. This is also evident by comparison of the GRASP surfaces for IL-4 and IL-13 colored by electrostatic potential (not shown). A distinct surface is presented to IL-4Rα by the two proteins, where IL-4 presents a relatively higher negative charged surface compared to IL-13. Conversely, the IL-13 surface is more hydrophobic compared to IL-4 with some positive charge characteristics. This analysis implies that while some key interactions consistent with the IL-4/IL-4R complex are present, the IL-13/IL-4Rα model predicts that some re-arrangement of the IL-13 interaction with IL-4Rα is required to optimize the secondary interactions and accommodate the residue substitutions between IL-13 and IL-4.

The apparent sub-optimal interface between IL-13 and IL-4Rα based on the IL-4/IL-4Rα complex appears consistent with both the experimental affinity of IL-13 with IL-4Rα and the assembly mechanism of IL-4 with IL-4R. A sequential order of binding of IL-4 to IL-4R has been previously proposed (Kondo et al., 1993, Russell et al., 1993). First, IL-4 binds the IL-4Rα chain with high affinity (K_{d}=20-300 pM). The resulting complex then recruits the common γC chain to form the signaling heterodimer. Upon complex formation with IL-4Rα chain, IL-4 incurs a conformational change localized in the putative γC chain binding site (Wang et al., Kruse et al., Letzelter et al., Hage et al.). Presumably, the observed IL-4 conformational change is required to bind the γC chain binding. A similar mechanism appears consistent with the interaction of IL-13 with its receptor.

IL-13 does not bind IL-4R or the IL-4Rα chain in the absence of the IL-13 binding chain (Zurawski et al., 1993), but binds to the IL-13 binding chain (IL-13Rα1) with relatively high affinity (K_{d}˜4 nM). Following the sequential binding mechanism proposed for IL-4, IL-13 would appear to first bind the IL-13 binding chain. The resulting complex then recruits the IL-4Rα chain to from the signaling heterodimer. Upon complex formation with the IL-13 binding chain, IL-13 would presumably incur a conformational change that would allow it to bind IL-4Rα. Again, this conformational change would probably resemble the change observed with IL-4 and result in a subtle re-arrangement in IL-13 helices α_{A} and α_{C}. Since the IL-13/IL-4Rα model reveals that the basic interaction network consistent with the IL-4/IL-4Rα is present, presumably only a modest modification in the helical packing would establish a comparable binding interface with IL-4/IL-4Rα complex and improve the affinity of IL-13 with IL-4Rα.

TABLE 1
Structural Statistics and Atomic r.m.s. Differences

| | <SA> | \overline{(SA)}ᵣ |
|---|---|---|
| A. Structural Statistics | | |
| r.m.s. deviations from experimental | | |
| distance restraints (Å)^{a} | | |
| all (2248) | 0.014 ± 0.002 | 0.016 |
| interresidue sequential | 0.011 ± 0.004 | 0.012 |
| (\|i-j\| = 1) (624) | | |
| interresidue short range | 0.015 ± 0.003 | 0.018 |
| (1 < \|i-j\| < 5) (607) | | |
| interresidue long-range | 0.016 ± 0.002 | 0.021 |
| (\|i-j\| > 5) (530) | | |
| intraresidue (437) | 0.007 ± 0.004 | 0.005 |
| H-bonds (50)^{b} | 0.031 ± 0.006 | 0.026 |
| r.m.s. deviation from exptl dihedral | 0.221 ± 0.053 | 0.186 |
| restraints (deg) (299)^{c,d} | | |
| r.m.s. deviation from exptl C^{α} | 0.95 ± 0.03 | 0.94 |
| restraints (ppm) (104) | | |
| r.m.s. deviation from exptl C^{β} | 0.78 ± 0.04 | 0.78 |
| restraints (ppm) (101) | | |
| r.m.s. deviation from 3J_{NHα} | 0.61 ± 0.02 | 0.58 |
| restraints (Hz) (96) | | |
| F_{NOE} (kcal mol⁻¹)^{d} | 22.3 ± 5.9 | 28.5 |
| Fₜₒᵣ (kcal mol⁻¹)^{d} | 0.95 ± 0.46 | 0.64 |
| Fᵣₑₚₑₗ (kcal mol⁻¹)^{d} | 22.5 ± 2.1 | 14.4 |
| F_{L-J} (kcal mol⁻¹)^{e} | −423 ± 8 | −408 |
| deviations from idealized | | |
| covalent geometry | | |
| bonds (Å) (1783) | 0.003 ± 0 | 0.004 |
| angles (deg) (3240) | 0.455 ± 0.011 | 0.523 |
| impropers (deg) (901)^{f} | 0.437 ± 0.039 | 0.396 |
| PROCHECK^{g} | | |
| Overall G-Factor | 0.19 ± 0.02 | 0.15 |
| % Residues in most favorable region | 90.5 ± 1.4 | 89.9 |
| of Ramachandran plot | | |
| % Residues in disallowed region | 0.0 ± 0.0 | 0.0 |
| of Ramachandran plot | | |
| H-bond energy | 0.85 ± 0.06 | 0.90 |
| Number of bad contacts/100 residues | 2.6 ± 1.5 | 1.8 |
| B. Atomic r.m.s. Differences (Å) | | |

| | | | | | ordered |
|---|---|---|---|---|---|
| | Residues 1-113 | | secondary structure^{h} | | side chain |
| | backbone | all | backbone | all | all |
| | atoms | atoms | atoms | atoms | atoms |
| <SA> vs \overline{SA} | 0.43 ± | 0.81 ± | 0.22 ± | 0.65 ± | 0.47 ± |
| | 0.04 | 0.06 | 0.03 | 0.06 | 0.04 |
| <SA> vs \overline{(SA)}ᵣ | 0.45 ± | 0.90 ± | 0.24 ± | 0.73 ± | 0.51 ± |
| | 0.04 | 0.07 | 0.03 | 0.08 | 0.04 |
| \overline{(SA)}ᵣ vs \overline{SA} | 0.15 | 0.38 | 0.10 | 0.32 | 0.20 |

The notation of the structures is as follows: <SA> are the final 30 simulated annealing structures; \overline{SA} is the mean structure obtained by averaging the coordinates of the individual SA structures best fit to each; and (\overline{SA})ᵣ is the restrained minimized mean structure obtained by restrained minimization of the mean structure \overline{SA} (Nilges et al., 1988). The number of terms for the various restraints is given in parentheses. a None of the structures exhibited distance violations greater than 0.2 Å or dihedral angle violations greater than 1°. b For backbone NH—CO hydrogen bond there are two restraints: r_{NH—O}=1.5-2.3 Å and r_{N—O}=2.5-3.3 Å. All hydrogen bonds involve slowly exchanging NH protons. c The torsion angle restraints comprise 104 φ, 105 ψ, 66 χ1, and 24 χ2 restraints. d The values of the square-well NOE (F_{NOE}) and torsion angle (Fₜₒᵣ) potentials [cf. eqs 2 and 3 in Clore et al., (1986)] are calculated with force constants of 50 kcal mol⁻¹ Å⁻² and 200 kcal mol⁻¹ rad⁻², respectively. The value of the quartic van der Waals repulsion term (Fᵣₑₚ) [cf. eq 5 in Nilges et al. (1988)] is calculated with a force constant of 4 kcal mol⁻¹ Å⁻⁴ with the hard-sphere van der Waals radius set to 0.8 times the standard values used in the CHARMM (Brooks et al., 1983) emperical energy function (Nilges et al., 1988, Nilges et al., 1988, Nilges et al., 1988). e E_{L-J} is the Lennard-Jones-van der Waals energy calculated with the CHARMM emperical energy function and is not included in the target function for simulated annealing or restrained minimization. f The improper torsion restraints serve to maintain planarity and chirality. g These were calculated using the PROCHECK program (Laskowski et al., 1996). h The residues in the regular secondary structure are: 6-22 (α_{A}), 43-52(α_{B}), 59-70(α_{C}), 92-108(α_{D}), 33-35(β₁) and 89-91(β_{II}).

### REFERENCES

Archer, S. J., Ikura, M. Torchia, D. A., and Bax, A. (1991). An alternative 3D NMR technique for correlating backbone nitrogen-15 with side chain Hα resonances in larger proteins, J Magn Reson 95, 636-641.

Bax, A., and Pochapsky, S. S. (1992). Optimized Recording of Heteronuclear Multidimensional NMR Spectra Using Pulsed Field Gradients, Journal of Magnetic Resonance 99, 638-643.

Bax, A., Vuister, G. W., Grzesiek, S., and Delaglio (1994). Measurement of homo- and heteronuclear J couplings from quantitative J correlation, Methods Enzymol 239, 79-105.

Brooks, B. R., Bruccoleri, R. E., Olafson, B. D., States, D. J., Swaminathan, S., and Karplus, M. (1983). CHARMM: a program for macromolecular energy, minimization, and dynamics calculations, J Comput Chem 4, 187-217.

Brunger, A. T. (1993). X-PLOR Version 3.1 Manual (New Haven, Conn., Yale University). Callard, R. E., Matthews, D. J., and Hibbert, L. (1996). IL-4 and IL-13 receptors: are they one and the same?, Immunol Today 17, 108-110.

Clore, G. M., Appella, E., Yamada, M., Matsushima, K., and Gronenbom, A. M. (1990). Three-dimensional structure of interleukin 8 in solution, Biochemistry 29, 1689-1696.

Clore, G. M., and Gronenborn, A. M. (1994). Multidimensional heteronuclear nuclear magnetic resonance of proteins, Methods Enzymol 239, 349-362.

Clore, G. M., Nilges, M., Sukumaran, D. K., Bruenger, A. T., Karplus, M., and Gronenborn, A. M. (1986). The three-dimensional structure of α1-purothionin in solution: combined use of nuclear magnetic resonance, distance geometry and restrained molecular dynamics, EMBO J 5, 2729-2735.

Cornilescu, G., Delaglio, F., and Bax, A. (1999). Protein backbone angle restraints from searching a database for chemical shift and sequence homology, J Biomol NMR 13, 289-302.

Corry, D. B. (1999). IL-13 in allergy: home at last, Curr Opin Immunol 11, 610-614.

De Vries, J. E. (1994). Inhibition of IL-4- and IL-13-induced IgE synthesis by an IL-4 mutant protein, Arb Paul Ehrlich Inst 87, 275-282.

De Vries, J. E. (1998). The role of IL-13 and its receptor in allergy and inflammatory responses, J Allergy Clin Immunol 102, 165-169.

Delaglio, F., Grzesiek, S., Vuister, G. W., Zhu, G., Pfeifer, J., and Bax, A. (1995). NMRPipe: A multidimensional spectral processing system based on UNIX pipes, J Biomol NMR 6, 277-293.

Finkelman, F. D., Wynn, T. A., Donaldson, D. D., and Urban, J. F., Jr. (1999). The role of IL-13 in helminth-induced inflammation and protective immunity against nematode infections, Curr Opin Immunol 11, 420-426.

Garrett, D. S., Kuszewski, J., Hancock, T. J., Lodi, P. J., Vuister, G. W., Gronenbom, A. M., and Clore, G. M. (1994). The impact of direct refinement against three-bond HN—CH coupling constants on protein structure determination by NMR, J Magn Reson, Ser B 104, 99-103.

Garrett, D. S., Powers, R., Gronenbom, A. M., and Clore, G. M. (1991). A common sense approach to peak picking in two-, three-, and four-dimensional spectra using automatic computer analysis of contour diagrams, J Magn Reson 95, 214-220.

Gessner, A., and Rollinghoff, M. (2000). Biologic functions and signaling of the interleukin-4 receptor complexes, Immunobiology 201, 285-307.

Grzesiek, S., Kuboniwa, H., Hinck, A. P., and Bax, A. (1995). Multiple-Quantum Line Narrowing for Measurement of Hα.-Hβ J Couplings in Isotopically Enriched Proteins, J Am Chem Soc 117, 5312-5315.

Hage, T., Sebald, W., and Reinemer, P. (1999). Crystal structure of the interleukin-4/receptor a chain complex reveals a mosaic binding interface, Cell 97, 271-281.

Ikura, M., Kay, L. E., Tschudin, R., and Bax, A. (1990). Three-dimensional NOESY-HMQC spectroscopy of a carbon-13-labeled protein, J Magn Reson 86, 204-209.

Kondo, M., Takeshita, T., Ishii, N., Nakamura, M., Watanabe, S., Arai, K., and Sugamura, K. (1993). Sharing of the interleukin-2 (IL-2) receptor g chain between receptors for IL-2 and IL-4, Science 262, 1874-1877.

Kruse, N., Shen, B. J., Arnold, S., Tony, H. P., Mueller, T., and Sebald, W. (1993). Two distinct functional sites of human interleukin 4 are identified by variants impaired in either receptor binding or receptor activation, Embo J 12, 5121-5129.

Kuszewski, J., Gronenbom, A. M., and Clore, G. M. (1996). Improving the quality of NMR and crystallographic protein structures by means of a conformational database potential derived from structure databases, Protein Sci 5, 1067-1080.

Kuszewski, J., Gronenbom, A. M., and Clore, G. M. (1997). Improvements and extensions in the conformational database potential for the refinement of NMR and x-ray structures of proteins and nucleic acids, J Magn Reson 125, 171-177.

Kuszewski, J., Gronenbom, A. M., and Clore, G. M. (1999). Improving the Packing and Accuracy of NMR Structures with a Pseudopotential for the Radius of Gyration, J Am Chem Soc 121, 2337-2338.

Kuszewski, J., Qin, J., Gronenbom, A. M., and Clore, G. M. (1995). The impact of direct refinement against 13Cα and 13Cβ chemical shifts on protein structure determination by NMR, J Magn Reson, Ser B 106, 92-96.

Laskowski, R., Rullmannn, J., MacArthur, M., Kaptein, R., and J M, T. (1996). AQUA and PROCHECK-NMR: programs for checking the quality of protein structures solved by NMR., Biomol NMR 8, 477-486.

Letzelter, F., Wang, Y., and Sebald, W. (1998). The interleukin-4 site-2 epitope determining binding of the common receptor g chain, Eur J Biochem 257, 11-20.

Marion, D., Driscoll, P. C., Kay, L. E., Wingfield, P. T., Bax, A., Gronenbom, A. M., and Clore, G. M. (1989). Overcoming the overlap problem in the assignment of proton NMR spectra of larger proteins by use of three-dimensional heteronuclear proton-nitrogen-15 Hartmann-Hahn-multiple quantum coherence and nuclear Overhauser-multiple quantum coherence spectroscopy: application to interleukin 1β, Biochemistry 28, 6150-6156.

Nilges, M., Clore, G. M., and Gronenbom, A. M. (1988a). Determination of three-dimensional structures of proteins from interproton distance data by dynamical simulated annealing from a random array of atoms. Circumventing problems associated with folding, Febs Lett 239, 129-136.

Nilges, M., Clore, G. M., and Gronenborn, A. M. (1988b). Determination of three-dimensional structures of proteins from interproton distance data by hybrid distance geometry-dynamical stimulated annealing calculations, Febs Lett 229, 317-324.

Nilges, M., Gronenbom, A. M., Bruenger, A. T., and Clore, G. M. (1988c). Determination of three-dimensional structures of proteins by simulated annealing with interproton distance restraints. Application to crambin, potato carboxypeptidase inhibitor and barley serine proteinase inhibitor 2, Protein Eng 2, 27-38.

Oshima, Y., Joshi, B. H., and Puri, R. K. (2000). Conversion of interleukin-13 into a high affinity agonist by a single amino acid substitution, J Biol Chem 275, 14375-14380.

Powers, R., Garrett, D. S., March, C. J., Frieden, E. A., Gronenbom, A. M., and Clore, G. M. (1992). Three-dimensional solution structure of human interleukin-4 by multidimensional heteronuclear magnetic resonance spectroscopy, Science 256, 1673-1677.

Powers, R., Garrett, D. S., March, C. J., Frieden, E. A., Gronenborn, A. M., and Clore, G. M. (1993). The high-resolution, three-dimensional solution structure of human interleukin-4 determined by multidimensional heteronuclear magnetic resonance spectroscopy, Biochemistry 32, 6744-6762.

Russell, S. M., Keegan, A. D., Harada, N., Nakamura, Y., Noguchi, M., Leland, P., Friedmann, M. C., Miyajima, A., Puri, R. K., and et al. (1993). Interleukin-2 receptor g chain: a functional component of the interleukin-4 receptor, Science 262, 1880-1883.

Shirakawa, T. T., Deichmann, K. A. K. A., Izuhara, K. K., Mao, X. Q. X. Q., Adra, C. N. C. N., and Hopkin, J. M. J. M. (2000). Atopy and asthma: genetic variants of IL-4 and IL-13 signaling, Immunol Today 21, 60-64.

Smith, L. J., Redfield, C., Boyd, J., Lawrence, G. M. P., Edwards, R. G., Smith, R. A. G., and Dobson, C. M. (1992). Human interleukin 4. The solution structure of a four-helix bundle protein, J Mol Biol 224, 899-904.

Smith, L. J., Redfield, C., Smith, R. A. G., Dobson, C. M., Clore, G. M., Gronenbom, A. M., Walter, M. R., Naganbushan, T. L., and Wlodawer, A. (1994). Comparison of four independently determined structures of human recombinant interleukin-4, Nat Struct Biol 1, 301-310.

Sprang, S. R., and Bazan, J. F. (1993). Cytokine structural taxonomy and mechanisms of receptor engagement, Curr Opin Struct Biol 3, 815-827.

Thompson, J. P., and Debinski, W. (1999). Mutants of interleukin 13 with altered reactivity toward interleukin 13 receptors, J Biol Chem 274, 29944-29950.

Vuister, G. W., and Bax, A. (1993). Quantitative J correlation: a new approach for measuring homonuclear three-bond J(HNHα) coupling constants in ¹⁵N-enriched proteins, J Am Chem Soc 115, 7772-7777.

Walter, M. R., Cook, W. J., Zhao, B. G., Cameron, R. P., Jr., Ealick, S. E., Walter, R. L., Jr., Reichert, P., Nagabhushan, T. L., Trotta, P. P., and Bugg, C. E. (1992). Crystal structure of recombinant human interleukin-4, J Biol Chem 267, 20371-20376.

Wang, Y., Shen, B. -J., and Sebald, W. (1997). A mixed-charge pair in human interleukin 4 dominates high-affinity interaction with the receptor a chain, Proc Natl Acad Sci U S A 94, 1657-1662.

Wills-Karp, M., Luyimbazi, J., Xu, X., Schofield, B., Neben, T. Y., Karp, C. L., and Donaldson, D. D. (1998). Interleukin-13: central mediator of allergic asthma, Science 282, 2258-2261.

Wlodaver, A., Pavlovsky, A., and Gustchina, A. (1992). Crystal structure of human recombinant interleukin-4 at 2.25 A resolution, FEBS Lett 309, 59-64.

Wuthrich, K., Billeter, M., and Braun, W. (1983). Pseudo-structures for the 20 Common Amino Acids for Use in Studies of Protein Conformation by Measurements of Intramolecular Proton-Proton Distance Constraints with Nuclear Magnetic Resonance., J Mol Biol 169, 949-996.

Zulderweg, E. R. P., Boelens, R., and Kaptein, R. (1985). Stereospecific assignments of proton-NMR methyl lines and conformation of valyl residues in the lac repressor headpiece, Biopolymers 24, 601-611.

Zuiderweg, E. R. P., and Fesik, S. W. (1989). Heteronuclear three-dimensional NMR spectroscopy of the inflammatory protein C5a, Biochemistry 28, 2387-2391.

Zuiderweg, E. R. P., McIntosh, L. P., Dahlquist, F. W., and Fesik, S. W. (1990). Three-dimensional carbon-13-resolved proton NOE spectroscopy of uniformly carbon-13-labeled proteins for the NMR assignment and structure determination of larger molecules, J Magn Reson 86, 210-216.

Zurawski, S. M., Vega, F., Jr., Huyghe, B., and Zurawski, G. (1993). Receptors for interleukin-13 and interleukin-4 are complex and share a novel component that functions in signal transduction, Embo J 12, 2663-2670.

All publications mentioned herein above, whether to issued patents, pending applications, published articles, protein structure deposits, or otherwise, are hereby incorporated by reference in their entirety. While the foregoing invention has been described in some detail for purposes of clarity and understanding, it will be appreciated by one skilled in the art from a reading of the disclosure that various changes in form and detail can be made without departing from the true scope of the invention in the appended claims.

What is claimed is:

1. A solution comprising interleukin-13 (IL-13), wherein IL-13 comprises amino acid residues 1-113 of FIG. 2, IL-13 is either unlabeled, ¹⁵N enriched or ¹⁵N,¹³C enriched, IL-13 comprises four alpha helices αA, αB, αC and αD, and two beta strands β1 and β2, and αA comprises amino acid residues P6-Q22 of IL-13, β1 comprises M33-W35 of IL-13, αB comprises amino acid residues M43-152 of IL-13, αC comprises amino acid residues A59-F70 of IL-13, β2 comprises amino acid residues K89-E91 of IL-13, and αD comprises amino acid residues V92-R108 of IL-13.

2. The solution of claim 1, wherein IL-13 has the structure defined by the relative structural coordinates according to FIG. 8, ± a root mean square deviation from the conserved backbone atoms of said amino acids of not more than 1.5 Å.

3. The solution of claim 1, wherein IL-13 has the structure defined by the relative structural coordinates according to FIG. 8, ± a root mean square deviation from the conserved backbone atoms of said amino acids of not more than 1.0 Å.

4. The solution of claim 1, wherein IL-13 has the structure defined by the relative structural coordinates according to FIG. 8, ± a root mean square deviation from the conserved backbone atoms of said amino acids of not more than 0.5 Å.

5. A structural model of IL-13 comprising the relative structural coordinates according to FIG. 8 or 9 of IL-13, ± a root mean square deviation from the conserved backbone atoms of said amino acids of not more than 1.5 Å.

6. The model of claim 5, wherein the ± a root mean square deviation from the conserved backbone atoms of said amino acids is not more than 1.0 Å.

7. The model of claim 5, wherein the ± a root mean square deviation from the conserved backbone atoms of said amino acids is not more than 0.5 Å.

8. An active site of IL-13, wherein said active site is characterized by a three dimensional structure comprising the relative structural coordinates of amino acid residues A9, E12, E15, E16 and M66 of IL-13 according to FIG. 8 or 9, ± a root mean square deviation from the conserved backbone atoms of said amino acids of not more than 1.5 Å.

9. The active site of claim 8, wherein the ± a root mean square deviation from the conserved backbone atoms of said amino acids is not more than 1.0 Å.

10. The active site of claim 8, wherein the ± a root mean square deviation from the conserved backbone atoms of said amino acids is not more than 0.5 Å.

11. An active site of IL-13, wherein said active site is characterized by a three dimensional structure comprising the relative structural coordinates of amino acid residues I52, Q64, R65 and M66 of IL-13 according to FIG. 8 or 9, ± a root mean square deviation from the conserved backbone atoms of said amino acids of not more than 1.5 Å.

12. The active site of claim 11, wherein the ± a root mean square deviation from the conserved backbone atoms of said amino acids is not more than 1.0 Å.

13. The active site of claim 11, wherein the ± a root mean square deviation from the conserved backbone atoms of said amino acids is not more than 0.5 Å.

14. A method for designing an agent that interacts with IL-13, comprising the steps of:
(a) generating a three dimensional model of IL-13 using the relative structural coordinates of the amino acids of FIG. 8 or 9, ± a root mean square deviation from the conserved backbone atoms of said amino acids of not more than 1.5 Å; and
(b) employing said three-dimensional model to design an agent that interacts with IL-13.

15. The method of claim 14, wherein the ± a root mean square deviation from the conserved backbone atoms of said amino acids is not more than 1.0 Å.

16. The method of claim 14, wherein the ± a root mean square deviation from the conserved backbone atoms of said amino acids is not more than 0.5 Å.

17. The method of claim 14, wherein the agent is designed using an active site of IL-13.

18. The method of claim 17, wherein the active site comprises the relative structural coordinates of amino acid residues A9, E12, E15, E16 and M66 of IL-13 according to FIG. 8 or 9, ± a root mean square deviation from the conserved backbone atoms of said amino acids of not more than 1.5 Å.

19. The method of claim 18, wherein the ± a root mean square deviation from the conserved backbone atoms of said amino acids is not more than 1.0 Å.

20. The method of claim 18, wherein the ± a root mean square deviation from the conserved backbone atoms of said amino acids is not more than 0.5 Å.

21. The method of claim 17, wherein the active site comprises the relative structural coordinates of amino acid residues I52, Q64, R65 and M66 of IL-13 according to FIG. 8 or 9, ± a root mean square deviation from the conserved backbone atoms of said amino acids of not more than 1.5 Å.

22. The method of claim 21, wherein the ± a root mean square deviation from the conserved backbone atoms of said amino acids is not more than 1.0 Å.

23. The method of claim 21, wherein the ± a root mean square deviation from the conserved backbone atoms of said amino acids is not more than 0.5 Å.

24. The method according to claim 14, wherein the step of employing the three dimensional structure to design an agent comprises the steps of:
(a) identifying chemical entities or fragments capable of associating with IL-13; and
(b) assembling the identified chemical entities or fragments into a single molecule to provide the structure of the agent.

25. The method according to claim 14, wherein the agent is designed de novo.

26. The method according to claim 14, wherein the agent is designed from a known agent.

27. The method of claim 14, further comprising the step of obtaining or synthesizing the agent.

28. The method of claim 27, wherein the agent obtained or synthesized in is contacted with IL-13 in order to determine the effect the agent has on IL-13.

29. An agent designed by the method of claim 14.
